(19) United States
(12) Patent Application Publication
(10) Pub. No.: US 2025/0195693 A1
(43) Pub. Date: Jun. 19, 2025

(54) Compositions Comprising Adeno-Associated Virus Chimera Capsid Proteins and Methods of Using the Same

(71) Applicant: Duke University, Durham, NC (US)

(72) Inventors: Aravind Asokan, Durham, NC (US); Lawrence Patrick Havlik, Durham, NC (US)

(21) Appl. No.: 18/288,817

(22) PCT Filed: Apr. 29, 2022
(86) PCT No.: PCT/US22/27028

§ 371 Date: Oct. 29, 2023

Related U.S. Application Data
(60) Provisional application No. 63/182,433, filed on Apr. 30, 2021.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143; C12N 2750/14145

(57) ABSTRACT

Disclosed herein are AAV chimeric capsid proteins that confer an ability to evade neutralizing antibodies in humans. Disclosed herein are compositions comprising AAV chimeric capsid proteins and methods of making and using the AAV chimeric capsid proteins

### CROSS-REFERENCE TO RELATED APPLICATIONS

This application is a national phase application of International Application No. PCT/US22/27028 filed 29 Apr. 2022, which claims priority to U.S. Provisional Patent Application No. 63/182,433 filed 30 Apr. 2021, each of which is incorporated herein in its entirety.

### STATEMENT REGARDING FEDERAL FUNDING

This invention was made with Government support under Federal Grant No. R01HL089221 awarded by the National Institutes of Health. The Federal Government has certain rights to this invention.

### REFERENCE TO THE SEQUENCE LISTING

The Sequence Listing submitted 29 Apr. 2022 as a text file named “21_2008_WO_Sequence_Listing”, created on 29 Apr. 2022 and having a size of 543 kilobytes is hereby incorporated by reference pursuant to 37 C.F.R. § 1.52(e)(5).

### BACKGROUND OF THE INVENTION

Adeno-associated virus (AAV) vectors have become a leading platform for gene delivery for the treatment of a variety of diseases. Although there has been clinical success using AAV gene therapies, limitations and challenges associated with use of this gene delivery platform remain. Gene therapy with vectors (viral or non-viral) is sometimes complicated because of an immune response against the vector carrying the gene. The plasmids used for nonviral gene therapy, alone or in combination with liposomes or electrotransfer, can stimulate immune responses. Viral vectors are the most likely to induce an immune response, especially those, like adenovirus and AAV, which express immunogenic epitopes within the organism.

Immunity against vectors and their contents can substantially reduce the efficiency of gene therapy. A strong immune response against the constituents of the vector or the transgene leads to rejection of the cells infected by the vector and, therefore, to a reduction in the duration of expression of the therapeutic protein. With 30-80% of adult humans producing antibodies against various AAVs, this pre-existing immunity is a significant challenge to AAV-based gene therapy. Likewise, AAV redosing is limited due to antibody cross-reactivity between AAV serotypes, which makes it difficult for gene therapy to be repeated in patients. Solving the problem of pre-existing immunity against AAV vectors is complicated by the polyclonal nature of the antibody response and the resulting requirement that broad changes be made over the surface of the AAV capsid to achieve full evasion, which is costly and time-consuming.

Thus, there remains an urgent need for improved AAV vectors for therapeutic gene delivery. Consequently, the present disclosure provides compositions for and methods of engineering chimeric AAV capsids capable of antibody escape. The compositions and methods disclosed herein comprise AAV isolates of non-primate origin and possess the potential to evade neutralizing antisera in humans.

### BRIEF SUMMARY OF THE DISCLOSURE

Disclosed herein is an adeno-associated virus (AAV) chimeric capsid protein, comprising: a non-mammalian VP1 or a fragment thereof; and an N-terminus of a mammalian VP1 or a fragment thereof, wherein the N-terminus of the mammalian VP1 replaces the N-terminus of the non-mammalian VP1.

Disclosed herein is an AAV chimeric capsid protein comprising a chimeric sauropsidan VP1, wherein the chimeric sauropsidan VP1 comprises an N-terminus of VP1 of a mammalian AAV.

Disclosed herein is an AAV chimeric capsid protein comprising the sequence set forth in any one of SEQ ID NO:01-SEQ ID NO:07.

Disclosed herein is an AAV chimeric capsid protein comprising a chimeric VP1/VP2 (partial). Disclosed herein is an AAV chimeric capsid protein comprising a chimeric VP2 (partial)/VP3. Disclosed herein is an AAV chimeric capsid protein, comprising a chimeric VP1/VP2 (partial) and a chimeric VP2 (partial)/VP3. Disclosed herein is an AAV chimeric capsid protein comprising the sequence set forth in any one of SEQ ID NO:08-SEQ ID NO:17.

Disclosed herein is an AAV capsid comprising an AAV chimeric capsid protein comprising a non-mammalian VP1 or a fragment thereof; and an N-terminus of a mammalian VP1 or a fragment thereof, wherein the N-terminus of the mammalian VP1 replaces the N-terminus of the non-mammalian VP1.

Disclosed herein is an AAV capsid comprising 60 VPs, wherein the 60 VPs comprise one or more copies of a disclosed AAV chimeric capsid protein and one or more copies of a disclosed non-chimeric capsid protein, and wherein the AAV capsid display higher gene transfer efficiency than an AAV capsid not having one or more copies of the AAV chimeric capsid protein.

Disclosed herein is an AAV capsid comprising a chimeric sauropsidan VP1, wherein the chimeric sauropsidan VP1 comprises an N-terminus of a mammalian AAV VP1.

Disclosed herein is an AAV capsid comprising an AAV chimeric capsid protein comprising a chimeric VP1/VP2 (partial). Disclosed herein is an AAV capsid comprising an AAV chimeric capsid protein comprising a chimeric VP2 (partial)/VP3. Disclosed herein is an AAV capsid comprising an AAV chimeric capsid protein comprising a chimeric VP1/VP2 (partial) and a chimeric VP2 (partial)/VP3.

Disclosed herein is a method of treating and/or preventing a genetic disease or disorder, the method comprising administering to a subject in need thereof an AAV vector, wherein, following the administering of the vector, the subject does not develop an antibody response to the AAV vector.

Disclosed herein is a method of treating and/or preventing a genetic disease or disorder, the method comprising administering to a subject in need thereof an AAV vector, wherein the expression of the encoded transgene can restore one or more aspects of cellular homeostasis and/or cellular functionality and/or metabolic dysregulation.

The present disclosure provides, in part, adeno-associated virus (AAV) chimera capsid proteins comprising three capsid viral proteins (VPs) wherein at least one VP or a fragment thereof shares similarity with a non-mammalian VP and at least one other VP or a fragment thereof shares similarity with a mammalian VP. In some aspects, the present disclosure provides adeno-associated virus (AAV) chimera capsid proteins comprising three capsid viral proteins (VPs) wherein at least one VP or a fragment thereof shares similarity with a reptile VP and at least one other VP or a fragment thereof shares similarity with a primate VP.

An aspect of the present disclosure provides for AAV chimera capsid proteins comprising three capsid viral proteins (VPs) wherein at least one VP or a fragment thereof shares at least 85% amino acid sequence similarity with a non-mammalian (e.g., reptile) VP and at least one other VP or a fragment thereof shares at least 85% amino acid sequence similarity with a mammalian (e.g., primate) VP. In some aspects, AAV chimera capsid proteins herein may have a VP1 sharing at least 85% amino acid sequence similarity with a mammalian (e.g., primate) VP. In some aspects, AAV chimera capsid proteins herein may have a VP3 sharing at least 85% amino acid sequence similarity with a non-mammalian (e.g., reptile) VP.

In some aspects, AAV chimera capsid proteins herein may have a VP1 and a fragment of VP2 that share at least 85% amino acid sequence similarity with a mammalian (e.g., primate) VP. In accordance with some aspects herein, AAV chimera capsid proteins comprising a VP1 and a fragment of VP2 that share at least 85% amino acid sequence similarity with a mammalian (e.g., primate) VP may be any one of the sequences set forth in SEQ ID NO:29-SEQ ID NO:34.

In some aspects, AAV chimera capsid proteins herein may have a VP3 and a fragment of VP2 that share at least 85% amino acid sequence similarity with a non-mammalian (e.g., reptile) VP. In accordance with some aspects herein, AAV chimera capsid proteins comprising a VP3 and a fragment of VP2 that share at least 85% amino acid sequence similarity with a non-mammalian (e.g., reptile) VP may be any one of the sequences set forth in SEQ ID NO:18-SEQ ID NO: 28.

In some aspects, AAV chimera capsid proteins herein may have a VP1 and a fragment of VP2 comprising any one of the sequences set forth in SEQ ID NO:29-SEQ ID NO:34; and a VP3 and a fragment of VP2 comprising any one of the sequences set forth in SEQ ID NO:18-SEQ ID NO:28. In some aspects, AAV chimera capsid proteins herein may have any one of the sequences set forth in SEQ ID NO:08-SEQ ID NO:17.

In some aspects, AAV chimera capsid proteins herein may have at least one amino acid sequence from one or more AAV serotypes. In accordance with some aspects herein, AAV chimera capsid proteins herein having at least one amino acid sequence from one or more AAV serotypes may have one or more AAV serotypes comprising AAV2, AAV8, AAV9, or any combination thereof.

In some aspects, AAV chimera capsid proteins herein may be comprised of at least 60 VP copies derived from any of the chimera capsid proteins herein in any combination thereof.

Another aspect of the present disclosure provides for AAV vectors comprising a polynucleotide encoding any of the chimera capsid proteins disclosed herein and methods of making thereof. In some aspects, AAV vectors herein may further comprise a polynucleotide encoding at least one transgene of interest. In some aspects, AAV vectors herein may have less susceptibility to antibody-mediated neutralization compared to naturally isolated AAV vectors. In some aspects, AAV vectors herein may have less susceptibility to antibody-mediated neutralization compared to AAV vectors derived from a mammalian AAV capsid protein alone. In some aspects, AAV vectors herein may have a higher titer compared to naturally isolated AAV vectors. In some aspects, AAV vectors herein may have a higher titer compared to AAV vectors derived from a mammalian AAV capsid protein alone. In some aspects, AAV vectors herein may have a higher gene transfer efficiency compared to naturally isolated AAV vectors. In some aspects, AAV vectors herein may have a higher gene transfer efficiency compared to AAV vectors derived from a mammalian AAV capsid protein alone

Another aspect of the present disclosure provides for AAV particles comprising any of the chimera capsid proteins disclosed herein and methods of making thereof. In some aspects, AAV particles herein may further comprise a polynucleotide encoding at least one transgene of interest.

Another aspect of the present disclosure provides for pharmaceutical compositions comprising any of the chimera capsid proteins, AAV vectors, and/or AAV particles disclosed herein. In some aspects, pharmaceutical compositions herein may further comprise at least one pharmaceutically acceptable carrier.

Another aspect of the present disclosure provides for methods of introducing a gene into a target cell. In some aspects, the methods of introducing a gene into a target cell herein may include contacting the target cell with any of the chimera capsid proteins, AAV vectors, AAV particles, and/or pharmaceutical compositions disclosed herein. In some aspects, methods herein can deliver one or more transgenes to a target cell in a subject, the methods comprising administering to the subject any of the chimera capsid proteins, AAV vectors, AAV particles, and/or pharmaceutical compositions disclosed herein. In some aspects, methods herein can comprise administering to a subject any of the chimera capsid proteins, AAV vectors, AAV particles, and/or pharmaceutical compositions disclosed herein after at least one prior dosage of the subject with an AAV vector composed of mammalian AAV VPs alone. In some aspects, any of the compositions or AAV particles disclosed herein can be administered to a subject by intramuscular injection, intravenous injection, intracoronary injection, intraarterial injection, or any combination thereof.

Another aspect of the present disclosure provides for methods of generating recombinant adeno-associated viruses (AAVs) comprising a chimera capsid protein as disclosed herein. In some aspects, these methods can comprise one or more steps of culturing a host cell which may contain: (a) a polynucleotide encoding a chimera capsid protein disclosed herein; (b) a transgene; and/or (c) sufficient helper functions to permit packaging of the transgene into the AAV capsid.

### BRIEF DESCRIPTION OF THE DRAWINGS

The following drawings form part of the present specification and are included to further demonstrate certain aspects of the present disclosure, which can be better understood by reference to the drawings in combination with the detailed description of specific aspects presented herein.

FIG. 1A and FIG. 1B are schematics illustrating sequence alignments of AAV serotypes 1, 2, 3, 4, 5, 8, 9, avian DA-1, snake, and bearded dragon in accordance with aspects of the present disclosure. FIG. 1A shows a sequence alignment of VP1u before the PLA2 domain. FIG. 1B shows a sequence alignment of the VP2 region up to the start of AAP where the VP2 start and the end of the AAV8 sequence in BM8 are denoted by arrows and the BR3 NLS is denoted by a rectangle. Note that the reptile sequences in FIG. 1A and FIG. 1B are inside the dashed box and shared little homology to the primate sequences which are shown above the dashed box.

FIG. 2A and FIG. 2B are schematics illustrating the VP1, VP2, and VP3 regions of AAV8 and chimeric proteins having AAV8 and reptile AAV in accordance with aspects of the present disclosure. FIG. 2A shows a schematic of a BM8 chimeric capsid protein which was a fusion between AAV8 and avian AAV strain DA-1. VP1 and part of VP2 (until the AAP alternative reading frame) were from AAV8, while the remainder of VP2 and all of VP3 were from DA-1. FIG. 2B shows a schematic of SM8 and DM8 chimeric capsid proteins which were fusions between AAV8 and either snake AAV or bearded dragon AAV, respectively.

FIG. 3A and FIG. 3B are images illustrating Western blot analysis of DA-1 and BM8 in accordance with aspects of the present disclosure. FIG. 3A shows that DA-1 and BM8 were not recognized by anti-AAV2 antibody B1 (which binds at the C-terminus of VP3) but the antibody recognized AAV8. FIG. 3B shows that the anti-AAV2 antibody A1 was bound to VP1u, so AAV8 and BM8 were recognized whereas a leucine to phenylalanine change caused DA-1 to be less recognized by the antibody.

FIG. 4A and FIG. 4B are graphs illustrating transduction and antibody escape of BM8 in accordance with aspects of the present disclosure. FIG. 4A shows transduction of HEK293 cells with the indicated AAV luciferase vectors (AAV8, DA1, BM8) at 10,000 and 100,000 vg/cell. FIG. 4B shows inhibition of the indicated AAV luciferase vectors (AAV8, hum8, BM8) after pre-incubation with anti-AAV serum from a dog treated with AAV8 vector.

FIG. 5A and FIG. 5B are schematics and graphs illustrating transduction and antibody escape of mutated BM8 (BM8L and BM2) in accordance with aspects of the present disclosure. FIG. 5A shows a schematic depicting the mutated BM8 constructs, BM8L and BM2, where BM8L had VP1 from AAV8 and VP2 from avian AAV, and BM2 had the VP1/2 regions from AAV2 instead of AAV8. FIG. 5B shows transduction of U87 cells with the BM8 mutants where changing the VP1/2 region from AAV8 to AAV2 did not affect transduction (BM8 vs BM2), and reverting the VP2 portion back to avian AAV dropped transduction over 80% (BM8 vs BM8L).

FIG. 6 is a graph illustrating AAV transduction in the heart tissue of mice that were injected with AAV-luciferase vectors in accordance with aspects of the present disclosure. The mice were sacrificed four weeks post-injection and assayed for luciferase expression where AAV8 and BM8 showed transduction in the mouse heart while avian DA-1 did not show transduction in the tissue.

FIG. 7 is a graph illustrating AAV titers in accordance with aspects of the present disclosure where chimeric capsids showed increased titer compared to the natural, non-mammalian versions.

FIG. 8A and FIG. 8B are graphs illustrating transduction of A549 cells with AAV luciferase vectors having capsid proteins of either snake or bearded dragon AAVs fused with AAV8 in accordance with aspects of the present disclosure. FIG. 8A shows A549 cells transduced at 10k MOI where the bearded dragon AAV did not transduce above background at 10k MOI. FIG. 8B shows A549 cells transduced at 50k MOI where the chimeric capsid proteins transduced better than native AAV8 and SM8 transduced better than DM8.

FIG. 9A and FIG. 9B are graphs illustrating AAV transduction of either AAV9, SM8, or DM8 in the skeletal muscle (FIG. 9A) and liver (FIG. 9B) of mice that were injected with AAV-luciferase vectors, sacrificed four weeks post-injection, and assayed for luciferase expression in accordance with aspects of the present disclosure. In these mice, AAV9-luc had no expression when the animals had been immunized with AAV9-GFP and chimeric capsids were still able to transduce following AAV9-GFP immunization.

FIG. 10 shows that a phylogenetic analysis revealed that AAV strain DA-1 [YP_077183.1] to be among the most basal AAV groups. With only 60% pairwise amino acid identity between DA-1 and AAV8 VP3, the capsid surface topologies should be significantly different and should generate low antibody cross-reactivity.

FIG. 11 shows that DA-1 successfully assembled capsids and packaged genomes and demonstrated that packaged vector yields were about the same for AAV8 and DA-1.

FIG. 12 shows that DA-1 transduction was inhibited in a range of mammalian cell lines as demonstrated by a luciferase transduction assay.

FIG. 13A-FIG. 13C show that despite the failure to transduce mammalian cells, DA-1 bound to the cell surface of U87 cells (FIG. 13A) and was internalized at a level like that of AAV8 (FIG. 13B-FIG. 13C). These data indicated that the intracellular function of DA-1 was impaired.

FIG. 14 provides a schematic of AAV noting the VP1/2 unique region (linked to intracellular function of AAV) and the surface exposed residues in VP3.

FIG. 15 is a schematic that details the generation of various disclosed chimeric vectors and details the various substitutions including BM8, SM8, DM8, and BM2 as well as others.

FIG. 16 shows that BM8 robustly evaded anti-AAV8 antibodies in vitro as demonstrated by the transduction of HEK293 using a luciferase gene assay.

FIG. 17 shows that BM8 robustly evaded anti-AAV9 antibodies in vivo, demonstrating little antibody cross-reactivity, as measured by a luciferase assaying using transduced skeletal muscle (FIG. 17A), heart (FIG. 17B), and liver (FIG. 17C).

FIG. 18 shows that SM8 and DM8 transduced human cells and outperformed AAV8, demonstrating that the ability of VP1/2 swaps to rescue transduction was not limited to AAV8.

FIG. 19 shows that BM2 had a similar transduction profile in U87 cells as did BM8 and that BM2 and BM8 outperformed AAV8 as measured by luminescence.

FIG. 20 shows that swapping the first 41 amino acids of AAV8 onto DA-1 was sufficient to rescue transduction in U87 cells and demonstrating that a minimal N-terminus swapped outperformed the larger VP1u and VP1/2u swaps.

### DETAILED DESCRIPTION

Adeno-associated virus (AAV) vectors have become a leading platform for therapeutic gene delivery. Unfortunately, gene therapies having AAV vectors can sometimes be less effective than desired because of immune responses against the vector carrying the therapeutic gene (e.g., a transgene of interest). The present disclosure is based, at least in part, on the creation of chimera capsid proteins with previously unseen abilities to avoid pre-existing neutralizing antibodies generated against primate serotypes. Specifically, the disclosure provides for the development of a new class of AAV vectors, which could enable multiple rounds of dosing using different capsids and bring AAV gene therapy to patients previously disqualified by pre-existing immunity.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is +10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step (such as, for example, the AAV vector or the AAV chimera capsid protein).

As used herein, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing a disease or disorder (e.g., a genetic disease or disorder).

As used herein, the term “diagnosed” means having been subjected to an examination by a person of skill, for example, a physician, and found to have a condition that can be diagnosed or treated by one or more of the disclosed isolated nucleic acid molecules, disclosed AAV chimeric capsid proteins, disclosed vectors, disclosed pharmaceutical formulations, or a combination thereof, or by one or more of the disclosed methods. For example, “diagnosed with a disease or disorder” means having been subjected to an examination by a person of skill, for example, a physician, and found to have a condition (such as a genetic disease or disorder) that can be treated by one or more of the disclosed isolated nucleic acid molecules, disclosed AAV chimeric capsid proteins, disclosed vectors, disclosed pharmaceutical formulations, or a combination thereof, or by one or more of the disclosed methods. For example, “suspected of having a disease or disorder” can mean having been subjected to an examination by a person of skill, for example, a physician, and found to have a condition (such as a genetic disease or disorder) that can likely be treated by one or more of by one or more of the disclosed isolated nucleic acid molecules, disclosed AAV chimeric capsid proteins, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a disease or disorder (e.g., a genetic disease or disorder). In an aspect, a patient can refer to a subject that has been diagnosed with or is suspected of having a disease or disorder. In an aspect, a patient can refer to a subject that has been diagnosed with or is suspected of having a disease or disorder and is seeking treatment or receiving treatment for a disease or disorder.

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., a genetic disease or disorder) based upon an earlier diagnosis by a person of skill and thereafter subjected to treatment for the genetic disease or disorder. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder such as a genetic disease or disorder). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease.

For example, in an aspect, treating a disease or disorder can reduce the severity of an established a disease or disorder in a subject by 1%-100% as compared to a control (such as, for example, an individual not having a genetic disease or disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as a genetic disease or disorder). For example, treating a disease or disorder can reduce one or more symptoms of a disease or disorder in a subject by 1%-100% as compared to a control (such as, for example, an individual not having a genetic disease or disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder. It is understood that treatment does not necessarily refer to a cure or complete ablation or eradication of a disease or disorder. However, in an aspect, treatment can refer to a cure or complete ablation or eradication of a disease or disorder.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as a genetic disease or disorder) and/or related complication from progressing to that complication.

As used herein, the terms “administering” and “administration” refer to any method of providing one or more of the disclosed AAV vectors, disclosed AAV chimeric capsid proteins, disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed AAV vector, a disclosed pharmaceutical composition, a disclosed AAV chimeric capsid protein, a disclosed therapeutic agent, a disclosed immune modulator, a disclosed proteasome inhibitor, a disclosed small molecule, a disclosed endonuclease, a disclosed oligonucleotide, and/or a disclosed RNA therapeutic can comprise administration directly into the CNS or the PNS. Administration can be continuous or intermittent. Administration can comprise a combination of one or more route.

In an aspect, the skilled person can determine an efficacious dose, an efficacious schedule, and an efficacious route of administration for one or more of the disclosed AAV chimeric capsid proteins, the disclosed AAV vectors, disclosed pharmaceutical formulations, or a combination thereof to treat or prevent a disease or disorder (such as genetic disease or disorder). In an aspect, the skilled person can also alter, change, or modify an aspect of an administering step to improve efficacy of one or more of the disclosed AAV chimeric capsid proteins, the disclosed AAV vectors, disclosed pharmaceutical formulations, or a combination thereof.

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed AAV chimeric capsid proteins, the disclosed AAV vectors, disclosed pharmaceutical formulations, or a combination thereof administered to a subject, or by changing the frequency of administration of one or more of the disclosed AAV chimeric capsid proteins, the disclosed AAV vectors, disclosed pharmaceutical formulations, or a combination thereof to a subject, by changing the duration of time one or more of the disclosed AAV chimeric capsid proteins, the disclosed AAV vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during a common treatment schedule.

The term “contacting” as used herein refers to bringing one or more of the disclosed AAV vectors, disclosed pharmaceutical formulations, or a combination thereof together with a target area or intended target area in such a manner that the one or more of the disclosed AAV chimeric capsid proteins, the disclosed AAV vectors, disclosed pharmaceutical formulations, or a combination thereof exert an effect on the intended target or targeted area either directly or indirectly. A target area can comprise one or more cells, and in an aspect, one or more cells can be in a subject. A target area or intended target area can be one or more of a subject's organs (e.g., lungs, heart, liver, kidney, brain, etc.). In an aspect, a target area or intended target area can be any cell or any organ infected by a disease or disorder (such as a genetic disease or disorder). In an aspect, a target area or intended target area can be any organ, tissue, or cells that are affected by a disease or disorder (such as a genetic disease or disorder).

As used herein, “determining” can refer to measuring or ascertaining the presence and severity of a disease or disorder, such as, for example, a genetic disease or disorder. Methods and techniques used to determine the presence and/or severity of a disease or disorder are typically known to the medical arts. For example, the art is familiar with the ways to identify and/or diagnose the presence, severity, or both of a disease or disorder (such as, for example, a genetic disease or disorder).

As used herein, “effective amount” and “amount effective” can refer to an amount that is sufficient to achieve the desired result such as, for example, the treatment and/or prevention of a disease or disorder (e.g., a genetic disease or disorder) or a suspected disease or disorder. As used herein, the terms “effective amount” and “amount effective” can refer to an amount that is sufficient to achieve the desired an effect on an undesired condition (e.g., a disease or disorder). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed AAV chimeric capsid protein, a disclosed AAV vector, a disclosed pharmaceutical formulation, or any combination thereof; that (i) treats the particular disease, condition, or disorder (e.g., a genetic disease or disorder), (ii) attenuates, ameliorates, or eliminates one or more symptoms of the particular disease, condition, or disorder e.g., a genetic disease or disorder), or (iii) delays the onset of one or more symptoms of the particular disease, condition, or disorder described herein (e.g., a genetic disease or disorder). The specific therapeutically effective dose level for any particular patient will depend upon a variety of factors including the disorder being treated and the severity of the disorder; the disclosed AAV chimeric capsid proteins, the disclosed AAV vectors, and/or disclosed pharmaceutical formulations employed; the disclosed methods employed; the age, body weight, general health, sex and diet of the patient; the time of administration; the route of administration; the rate of excretion of the disclosed AAV chimeric capsid proteins, the disclosed AAV vectors, and/or disclosed pharmaceutical formulations employed; the duration of the treatment; drugs used in combination or coincidental with the disclosed AAV chimeric capsid proteins, the disclosed AAV vectors, and/or disclosed pharmaceutical formulations employed, and other like factors well known in the medical arts. For example, it is well within the skill of the art to start doses of the disclosed AAV vectors and/or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed AAV chimeric capsid proteins, the disclosed AAV vectors, and/or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

As used herein, “RNA therapeutics” can refer to the use of oligonucleotides to target RNA. RNA therapeutics can offer the promise of uniquely targeting the precise nucleic acids involved in a particular disease with greater specificity, improved potency, and decreased toxicity. This could be particularly powerful for genetic diseases where it is most advantageous to aim for the RNA as opposed to the protein. In an aspect, a therapeutic RNA can comprise one or more expression sequences. As known to the art, expression sequences can comprise an RNAi, shRNA, mRNA, non-coding RNA (ncRNA), an antisense such as an antisense RNA, miRNA, morpholino oligonucleotide, peptide-nucleic acid (PNA) or ssDNA (with natural, and modified nucleotides, including but not limited to, LNA, BNA, 2′-O-Me-RNA, 2′-MEO-RNA, 2′-F-RNA), or analog or conjugate thereof. In an aspect, a disclosed therapeutic RNA can comprise one or more long non-coding RNA (lncRNA), such as, for example, a long intergenic non-coding RNA (lincRNA), pre-transcript, pre-miRNA, pre-mRNA, competing endogenous RNA (ceRNA), small nuclear RNA (snRNA), small nucleolar RNA (snoRNA), pseudo-gene, rRNA, or tRNA. In an aspect, ncRNA can be piwi-interacting RNA (piRNA), primary miRNA (pri-miRNA), or premature miRNA (pre-miRNA). In an aspect, a disclosed therapeutic RNA or an RNA therapeutic can comprise antisense oligonucleotides (ASOs) that inhibit mRNA translation, oligonucleotides that function via RNA interference (RNAi) pathway, RNA molecules that behave like enzymes (ribozymes), RNA oligonucleotides that bind to proteins and other cellular molecules, and ASOs that bind to mRNA and form a structure that is recognized by RNase H resulting in cleavage of the mRNA target. In an aspect, RNA therapeutics can comprise RNAi and ASOs that inhibit mRNA translation. Generally speaking, as known to the art, RNAi operates sequence specifically and post-transcriptionally by activating ribonucleases which, along with other enzymes and complexes, coordinately degrade the RNA after the original RNA target has been cut into smaller pieces while antisense oligonucleotides bind to their target nucleic acid via Watson-Crick base pairing, and inhibit or alter gene expression via steric hindrance, splicing alterations, initiation of target degradation, or other events.

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition in a disclosed method.

As used herein, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

“Nucleic acid” or “oligonucleotide” or “polynucleotide” as used herein means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also as used herein, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, as used herein, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

A “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

Different nucleic acids or proteins having homology can be referred to as “homologues”. The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the disclosed compositions and disclosed methods can comprise homologues to the disclosed nucleotide sequences and/or disclosed polypeptide sequences.

As used herein, a “regulatory element” can refer to promoters, enhancers, internal ribosomal entry sites (IRES), and other expression control elements (e.g., transcription termination signals, such as polyadenylation signals and poly-U sequences). Regulatory elements can include those that direct constitutive expression of a nucleotide sequence in many types of host cells and those that direct expression of the nucleotide sequence only in certain host cells (e.g., tissue-specific regulatory sequences).

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter. In an aspect, a liver specific promoter can comprise about 845-bp and comprise the thyroid hormone-binding globulin promoter sequences (2382 to 13), two copies of α1-microglobulin/bikunin enhancer sequences (22,804 through 22,704), and a 71-bp leader sequence as described by Ill C R, et al. (1997).

Ubiquitous/constitutive promoters” are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “serotype” is a distinction used to refer to an AAV having a capsid that is serologically distinct from other AAV serotypes. Serologic distinctiveness can be determined by the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance. For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, a desired gene), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a disease or disorder (such as a genetic disease or disorder).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions

AAVs have become widely used for gene therapy applications, with several advantages over other viral vectors, including a lower toxicity and the availability of over 150 naturally occurring genotypes and serotypes. These serotypes differ in their tropism, and thus can target most tissues and cell types for gene delivery. Recombinant AAVs (rAAVs), packaging a gene of interest (GOI), have been successfully studied in clinical trials for the treatment of a wide variety of rare genetic disorders. In these applications, the GOI replaces the natural AAV genome for delivery to tissues or cells to treat a monogenic disease. In addition, rAAVs are widely used research tools for transgene expression in tissue culture and preclinical animal models.

AAV capsids consist of a total of 60 molecules of viral proteins (VPs); a mixture of the three overlapping gene products, VP1, VP2, and VP3, encoded by the cap open reading frame (ORF) and organized in T=1 icosahedral symmetry. The VPs are generated through alternative splicing of the mRNA and use of an alternate translational start codon. The VP3 (59-61 kDa, 524-544 aa) sequence is shared among all VPs and is referred to as the VP3 common region. VP2 (64-67 kDa, 580-601 aa) is approximately 57 aa longer than VP3 and the VP2 N-terminal region is referred to as the VP1/VP2 common region. VP1 (79-82 kDa, 713-738aa) is approximately 137 aa longer than VP2 and this region is called the VP1 unique (VP1u) region. The VP3 common region assembles the icosahedral capsid. The VP1u contains an essential phospholipase A2 (PLA2) enzyme, and VP1u and VP1/VP2 common region contain nuclear localization sequences (NLSs).

### 1. AAV Chimera Capsid Proteins

Disclosed herein is an adeno-associated virus (AAV) chimeric capsid protein, comprising: a non-mammalian VP1 or a fragment thereof; and an N-terminus of a mammalian VP1 or a fragment thereof, wherein the N-terminus of the mammalian VP1 replaces the N-terminus of the non-mammalian VP1.

In an aspect, a disclosed non-mammalian VP1 or a fragment thereof can comprise a sauropsidan VP1. In an aspect, a disclosed non-mammalian VP1 or a fragment thereof can comprise a reptilian VP1. In an aspect, a disclosed non-mammalian VP1 or a fragment thereof can comprise an avian VP1.

In an aspect, a disclosed N-terminus of the non-mammalian VP1 that is replaced by the N-terminus of the mammalian VP1 can comprise about 30 amino acids to about 80 amino acids, about 40 amino acids to about 70 amino acids, or about 50 amino acids to about 60 amino acids.

In an aspect, a disclosed N-terminus of the non-mammalian VP1 that is replaced by the N-terminus of the mammalian VP1 can comprise about 30 amino acids, about 40 amino acids, about 50 amino acids, about 60 amino acids, about 70 amino acids, or about 80 amino acids.

In an aspect, a disclosed N-terminus of the mammalian VP1 that replaces the N-terminus of the non-mammalian VP1 can comprise about 30 amino acids to about 80 amino acids, about 40 amino acids to about 70 amino acids, or about 50 amino acids to about 60 amino acids.

In an aspect, a disclosed N-terminus of the mammalian VP1 that replaces the N-terminus of the non-mammalian VP1 can comprise about 30 amino acids, about 40 amino acids, about 50 amino acids, about 60 amino acids, about 70 amino acids, or about 80 amino acids.

In an aspect, a disclosed sauropsidan VP1 or fragment thereof can comprise snake AAV VP1, bearded dragon parvovirus 2014 VP1, AAV MHH-05-2015 VP1, duck AAV G003-20-Australia-2020 VP1, goose parvovirus Yan-1 VP1, or goose parvovirus DY VP1.

In an aspect, a disclosed N-terminus of a mammalian VP1 or fragment thereof can comprise the N-terminus of AAV2 VP1, the N-terminus of AAV4 ATCC VR-646 VP1, the N-terminus of AAV5 VP1, the N-terminus of AAV6 VP1, the N-terminus of AAV8 VP1, the N-terminus of AAV9 VP1, or the N-terminus of California sea lion AAV1 1187 VP1.

In an aspect, a disclosed AAV chimeric capsid protein can comprise the sequence set forth in any one of SEQ ID NO:36-SEQ ID NO:40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO: 74-SEQ ID NO: 80, or SEQ ID NO: 82-SEQ ID NO:88.

In an aspect, a disclosed AAV chimeric capsid protein can comprise a sequence having about 70%, about 75%, about 80%, about 85%, about 90%, or about 95% identity to the sequence set forth in any one of SEQ ID NO:36-SEQ ID NO:40, SEQ ID NO:42-SEQ ID NO:48, SEQ ID NO: 50-SEQ ID NO:56, SEQ ID NO:58-SEQ ID NO:64, SEQ ID NO:66-SEQ ID NO:72, SEQ ID NO: 74-SEQ ID NO:80, or SEQ ID NO:82-SEQ ID NO:88.

In an aspect, a disclosed AAV chimeric capsid protein can comprise a sequence having about 96%, about 97%, about 98%, or about 99% identity to the sequence set forth in any one of SEQ ID NO:36-SEQ ID NO:40, SEQ ID NO:42-SEQ ID NO:48, SEQ ID NO:50-SEQ ID NO: 56, SEQ ID NO:58-SEQ ID NO:64, SEQ ID NO:66-SEQ ID NO:72, SEQ ID NO:74-SEQ ID NO:80, or SEQ ID NO:82-SEQ ID NO:88.

In an aspect, a disclosed VP1 sequence can comprise the VP1 sequence set forth in any one of the accession numbers set forth below in Table 1.

TABLE 1
Representative VP1 Sequences

YP_077183.1, YP_009154713.1, YP_068094.1, YP_009552824.1, QTE04019.1, QIP76499.1,
QIP76501.1, QIP76503.1, QIP76505.1, QUX80128.1, QQG62940.1, QQG62938.1,
QIP76507.1, QIP76509.1, QUX80132.1, NP_852781.1, QEJ80809.1, AXL64653.1,
ACU30842.1, AHK22793.1, QJB76077.1, QKN88753.1, QKN88757.1, QKN88759.1,
QKN88761.1, QKN88763.1, QKN88765.1, QKN88769.1, QKN88771.1, QKN88773.1,
QKN88776.1, QKE60687.1, QKN88779.1, QKN88781.1, UAJ21443.1, UAJ21439.1,
AAR24365.1, AAR24366.1, AAR24364.1, AAR24367.1, QOE76067.1, ALL25612.1,
QBS32945.2, QGW62429.1, ALL25608.1, AXG65513.1, ARF02874.1, AXG65518.1,
ALL25610.1, AKP07383.1, AKP07385.1, AAR24363.1, ACE95853.1, UKY81257.1,
AAR24358.1, AAR24359.1, AAR24360.1, AAR24361.1, AAR24362.1, QBC88388.1,
QNQ79071.1, ATX74769.1, QOE76065.1, QOE76062.1, BDB32779.1, AER25359.1,
ABU50783.1, ABP82770.1, AYW76676.1, AGO17640.1, AUO28606.1, AKP07377.1,
AKP07375.1, AAS16481.1, ABU50786.1, AXJ21640.1, QKX94711.1, QKX94709.1,
AEV89790.1, ANN12566.1, AOG75031.1, AOG75033.1, QOW77864.1, QOW77868.1,
QOW77866.1, AAL37722.1, ATU81352.1, QDH44047.1, QWE79644.1, QNQ79073.1,
ANX99769.1, AWA45288.1, AUO28608.1, AMK92591.1, QBS32947.1, QBS32949.1,
ALH24915.1, ABU50780.1, QNQ79075.1, QNQ79077.1, QNQ79079.1, QNQ79081.1,
QNQ79083.1, QLF80190.1, ATX74765.1, QLF80192.1, QLF80194.1, ATX74761.1,
ATX74763.1, AEL87778.1, ALH24916.1, AXG65520.1, ACE95855.1, NP_043515.1,
ALH24917.1, ALH24918.1, AGT62583.1, ALH24919.1, ALI16103.1, ALH24920.1,
CAA92575.1, QBZ68882.1, AKP07379.1, AKP07381.1, AKP07373.1, CAA52984.1,
YP_068411.1, AMO03313.1, AAR92460.1, AZA08014.1, QNT61180.1, QBY91916.1,
AIR74894.1, ANX99765.1, ANX99767.1, AYV89931.1, ANP94887.1, ASR91721.1, or
QMI57778.1

In an aspect, a disclosed VP1 sequence can comprise a VP1 sequence set forth in Table 1 and can be combined with an N-terminus sequence such as the N-terminus sequence set forth in Table 2.

TABLE 2
Representative N-Terminal Sequences

AAB95450.1, AAB95452.1, ABI16639.1, AKU89595.1, AKU89600.1, ATV81503.1,
NP_044927.1, QDH44082.1, QDH44228.1, QDH44235.1, QDH44284.1, QDH44305.1,
UMO75524.1, YP_009507367.1, YP_068409.1, YP_077180.1, or YP_680426.1.

In an aspect, a disclosed VP1 sequence can comprise a VP1 sequence set forth in any one of the accession numbers set forth Table 3.

TABLE 3
Representative VP1 Sequences

YP_077183.1, YP_009154713.1, YP_068094.1, YP_009552824.1, QTE04019.1, QIP76499.1,
QIP76501.1, QIP76503.1, QIP76505.1, QUX80128.1, QQG62940.1, QQG62938.1,
QIP76507.1, QIP76509.1, QUX80132.1, NP_852781.1, QEJ80809.1, AXL64653.1,
ACU30842.1, AHK22793.1, QJB76077.1, QKN88753.1, QKN88757.1, QKN88759.1,
QKN88761.1, QKN88763.1, QKN88765.1, QKN88769.1, QKN88771.1, QKN88773.1,
QKN88776.1, QKE60687.1, QKN88779.1, QKN88781.1, UAJ21443.1, UAJ21439.1,
AAR24365.1, AAR24366.1, AAR24364.1, AAR24367.1, QOE76067.1, ALL25612.1,
QBS32945.2, or QGW62429.1

In an aspect, a disclosed VP1 sequence can comprise a VP1 sequence set forth in Table 3 and can be combined with the N-terminus sequence set forth in QDH44326.1.

In an aspect, a disclosed VP1 sequence can comprise a VP1 sequence set forth in Table 4.

TABLE 4
Representative VP1 Sequences

AAL37722.1, AAR92460.1, AAS16481.1, ABU50780.1, ABU50786.1, ACE95855.1,
AEL87778.1, AEV89790.1, AGO17640.1, AGT62583.1, AIR74894.1, AKP07373.1,
AKP07375.1, AKP07377.1, AKP07379.1, AKP07381.1, ALH24915.1, ALH24916.1,
ALH24917.1, ALH24918.1, ALH24919.1, ALH24920.1, ALI16103.1, AMK92591.1,
AMO03313.1, ANN12566.1, ANP94887.1, ANX99765.1, ANX99767.1, ANX99769.1,
AOG75031.1, AOG75033.1, ASR91721.1, ATU81352.1, ATX74761.1, ATX74763.1,
ATX74765.1, AUO28606.1, AUO28608.1, AWA45288.1, AXG65520.1, AXJ21640.1,
AYV89931.1, AYW76676.1, AZA08014.1, CAA52984.1, CAA92575.1, NP_043515.1,
QBS32947.1, QBS32949.1, QBY91916.1, QBZ68882.1, QDH44047.1, QKX94709.1,
QKX94711.1, QLF80190.1, QLF80192.1, QLF80194.1, QMI57778.1, QNQ79073.1,
QNQ79075.1, QNQ79077.1, QNQ79079.1, QNQ79081.1, QNQ79083.1, QNT61180.1,
QOW77864.1, QOW77866.1, QOW77868.1, QWE79644.1, or YP_068411.1

In an aspect, a disclosed VP1 sequence can comprise a VP1 sequence set forth in Table 4 and can be combined with the N-terminus sequence set forth in QDX47270.1.

In an aspect, a disclosed VP1 sequence can comprise a VP1 sequence set forth in Table 5.

TABLE 5
Representative VP1 Sequences

AAL37722.1, AAR24358.1, AAR24359.1, AAR24360.1, AAR24361.1, AAR24362.1,
AAR24363.1, AAR24364.1, AAR24365.1, AAR24366.1, AAR24367.1, AAR92460.1,
AAS16481.1, ABP82770.1, ABU50780.1, ABU50783.1, ABU50786.1, ACE95853.1,
ACE95855.1, ACU30842.1, AEL87778.1, AER25359.1, AEV89790.1, AGO17640.1,
AGT62583.1, AHK22793.1, AIR74894.1, AKP07373.1, AKP07375.1, AKP07377.1,
AKP07379.1, AKP07381.1, AKP07383.1, AKP07385.1, ALH24915.1, ALH24916.1,
ALH24917.1, ALH24918.1, ALH24919.1, ALH24920.1, ALI16103.1, ALL25608.1,
ALL25610.1, ALL25612.1, AMK92591.1, AMO03313.1, ANN12566.1, ANP94887.1,
ANX99765.1, ANX99767.1, ANX99769.1, AOG75031.1, AOG75033.1, ARF02874.1,
ASR91721.1, ATU81352.1, ATX74761.1, ATX74763.1, ATX74765.1, ATX74769.1,
AUO28606.1, AUO28608.1, AWA45288.1, AXG65513.1, AXG65518.1, AXG65520.1,
AXJ21640.1, AXL64653.1, AYV89931.1, AYW76676.1, AZA08014.1, BDB32779.1,
CAA52984.1, CAA92575.1, NP_043515.1, NP_852781.1, QBC88388.1, QBS32945.2,
QBS32947.1, QBS32949.1, QBY91916.1, QBZ68882.1, QDH44047.1, QEJ80809.1,
QGW62429.1, QIP76499.1, QIP76501.1, QIP76503.1, QIP76505.1, QIP76507.1, QIP76509.1,
QJB76077.1, QKE60687.1, QKN88753.1, QKN88757.1, QKN88759.1, QKN88761.1,
QKN88763.1, QKN88765.1, QKN88769.1, QKN88771.1, QKN88773.1, QKN88776.1,
QKN88779.1, QKN88781.1, QKX94709.1, QKX94711.1, QLF80190.1, QLF80192.1,
QLF80194.1, QMI57778.1, QNQ79071.1, QNQ79073.1, QNQ79075.1, QNQ79077.1,
QNQ79079.1, QNQ79081.1, QNQ79083.1, QNT61180.1, QOE76062.1, QOE76065.1,
QOE76067.1, QOW77864.1, QOW77866.1, QOW77868.1, QQG62938.1, QQG62940.1,
QTE04019.1, QUX80128.1, QUX80132.1, QWE79644.1, UAJ21439.1, UAJ21443.1,
UKY81257.1, YP_009154713.1, YP_009552824.1, YP_068094.1, YP_068411.1, or
YP_077183.1

In an aspect, a disclosed VP1 sequence can comprise the sequence set forth in Table 5, and can be combined with the N-terminus sequence set forth in ATV81503.1.

Disclosed herein is an AAV chimeric capsid protein, comprising a chimeric sauropsidan VP1, wherein the chimeric sauropsidan VP1 comprises an N-terminus of VP1 of a mammalian AAV.

In an aspect, a disclosed AAV chimeric capsid protein can comprise the sequence set forth in any one of SEQ ID NO:36-SEQ ID NO:40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO: 74-SEQ ID NO:80, or SEQ ID NO:82-SEQ ID NO:88. In an aspect, a disclosed AAV chimeric capsid protein can comprise a sequence having about 70%, about 75%, about 80%, about 85%, about 90%, or about 95% identity to the sequence set forth in any one of SEQ ID NO:36-SEQ ID NO:40, SEQ ID NO:42-SEQ ID NO:48, SEQ ID NO:50-SEQ ID NO:56, SEQ ID NO: 58-SEQ ID NO:64, SEQ ID NO:66-SEQ ID NO: 72, SEQ ID NO:74-SEQ ID NO:80, or SEQ ID NO:82-SEQ ID NO:88. In an aspect, a disclosed AAV chimeric capsid protein can comprise a sequence having about 96%, about 97%, about 98%, or about 99% identity to the sequence set forth in any one of SEQ ID NO:36-SEQ ID NO:40, SEQ ID NO:42-SEQ ID NO: 48, SEQ ID NO:50-SEQ ID NO:56, SEQ ID NO:58-SEQ ID NO:64, SEQ ID NO:66-SEQ ID NO:72, SEQ ID NO: 74-SEQ ID NO: 80, or SEQ ID NO:82-SEQ ID NO:88.

Disclosed herein is an AAV chimeric capsid protein comprising the sequence set forth in any one of SEQ ID NO:01-SEQ ID NO:07. Disclosed herein is an AAV chimeric capsid protein comprising a sequence having about 70%, about 75%, about 80%, about 85%, about 90%, or about 95% identity to the sequence set forth in any one of SEQ ID NO:01-SEQ ID NO:07. Disclosed herein is an AAV chimeric capsid protein comprising a sequence having about 96%, about 97%, about 98%, or about 99% identity to the sequence set forth in any one of SEQ ID NO:01-SEQ ID NO: 07.

Disclosed herein is an AAV chimeric capsid protein comprising a chimeric VP1/VP2 (partial). In an aspect, a disclosed VP2 (partial) can comprise a primate VP2 (partial). In an aspect, a disclosed chimeric VP1/VP2 (partial) can comprise an AAV1 VP1/VP2 (partial), an AAV2 VP1/VP2 (partial), an AAV8 VP1/VP2 (partial), an AAV9 VP1/VP2 (partial), an AAVhu.37 VP1/VP2 (partial), or an AAVrh.10 VP1/VP2 (partial). In an aspect, a disclosed chimeric VP1/VP2 (partial) can comprise the sequence set forth in any one of SEQ ID NO:29-SEQ ID NO:34. In an aspect, a disclosed chimeric VP1/VP2 (partial) can comprise a sequence having about 70%, about 75%, about 80%, about 85%, about 90%, or about 95% identity to the sequence set forth in any one of SEQ ID ID NO: 29-SEQ ID NO:34. In an aspect, a disclosed chimeric VP1/VP2 (partial) can comprise a sequence having about 96%, about 97%, about 98%, or about 99% identity to the sequence set forth in any one of SEQ ID ID NO: 29-SEQ ID NO:34.

Disclosed herein is an AAV chimeric capsid protein comprising a chimeric VP2 (partial)/VP3. In an aspect, a disclosed VP3 can comprise a reptile VP3. In an aspect, a disclosed chimeric VP2 (partial)/VP3 can comprise an avian DA-1 VP2 (partial), a bearded dragon VP2 (partial), a snake VP2 (partial), an avian RSBR151R VP2 (partial), an avian YZ1 VP2 (partial), an avian ZN1 VP2 (partial), an avian BRDF12 VP2 (partial), a Muscovy duck parvovirus FM VP2 (partial), an avian VR865 VP2 (partial), a Muscovy duck MHH 05-2015 VP2 (partial), or a pacific black duck AAV VP2 (partial). In an aspect, a disclosed chimeric VP2 (partial)/VP3 can comprise the sequence set forth in any one of SEQ ID NO:18-SEQ ID NO:28. In an aspect, a disclosed chimeric VP2 (partial)/VP3 can comprise a sequence having about 70%, about 75%, about 80%, about 85%, about 90%, or about 95% identity to the sequence set forth in any one of SEQ ID ID NO: 18-SEQ ID NO:28. In an aspect, a disclosed chimeric VP2 (partial)/VP3 can comprise a sequence having about 96%, about 96%, about 98%, or about 99% identity to the sequence set forth in any one of SEQ ID ID NO: 18-SEQ ID NO:28.

Disclosed herein is an AAV chimeric capsid protein, comprising a chimeric VP1/VP2 (partial) and a chimeric VP2 (partial)/VP3. In an aspect, a disclosed chimeric VP1/VP2 (partial) can comprise an AAV1 VP1, an AAV2 VP2, an AAV8 VP2, an AAV9 VP, an AAVhu.37 VP1, or an AAVrh.10 VP1, and wherein the chimeric VP2 (partial)/VP3 comprises an avian DA-1 VP2 (partial), a bearded dragon VP2 (partial), a snake VP2 (partial), an avian RSBR151R VP2 (partial), an avian YZ1 VP2 (partial), an avian ZN1 VP2 (partial), an avian BRDF12 VP2 (partial), a Muscovy duck parvovirus FM VP2 (partial), an avian VR865 VP2 (partial), a Muscovy duck MHH 05-2015 VP2 (partial), or a pacific black duck AAV VP2 (partial).

In an aspect, a disclosed chimeric VP1/VP2 (partial) can comprise a primate VP2 (partial), and wherein the chimeric VP2 (partial)/VP3 comprises a reptile VP3. In an aspect, a disclosed chimeric VP1/VP2 (partial) can comprise the sequence set forth in any one of SEQ ID NO: 29-SEQ ID NO:34, and wherein the chimeric VP2 (partial)/VP3 comprises the sequence set forth in any one of SEQ ID NO: 18-SEQ ID NO:28. In an aspect, a disclosed chimeric VP1/VP2 (partial) can comprise a sequence having about 70%, about 75%, about 80%, about 85%, about 90%, or about 95% identity to the sequence set forth in any one of SEQ ID ID NO: 29-SEQ ID NO:34, and wherein the chimeric VP2 (partial)/VP3 comprises a sequence having about 70%, about 75%, about 80%, about 85%, about 90%, or about 95% identity to the sequence set forth in any one of SEQ ID ID NO: 18-SEQ ID NO:28. In an aspect, a disclosed chimeric VP1/VP2 (partial) can comprise a sequence having about 96%, about 96%, about 98%, or about 99% identity to the sequence set forth in any one of SEQ ID ID NO: 29-SEQ ID NO:34, and wherein the chimeric VP2 (partial)/VP3 comprises a sequence having about 96%, about 97%, about 98%, or about 99% identity to the sequence set forth in any one of SEQ ID ID NO: 18-SEQ ID NO:28.

Disclosed herein is an AAV chimeric capsid protein comprising the sequence set forth in any one of SEQ ID NO:08-SEQ ID NO:17. Disclosed herein is an AAV chimeric capsid protein comprising a sequence having about 70%, about 75%, about 80%, about 85%, about 90%, or about 95% identity to the sequence set forth in any one of SEQ ID NO:08-SEQ ID NO:17. Disclosed herein is an AAV chimeric capsid protein comprising a sequence having about 96%, about 97%, about 98%, or about 99% identity to the sequence set forth in any one of SEQ ID NO:08-SEQ ID NO: 17.

Disclosed herein is an AAV chimeric capsid protein comprising a chimeric sauropsidan VP1, wherein the chimeric sauropsidan VP1 comprises an N-terminus of a mammalian AAV VP1.

In an aspect, a disclosed chimeric sauropsidan VP1 or fragment thereof can comprise snake AAV VP1, bearded dragon parvovirus 2014 VP1, AAV MHH-05-2015 VP1, duck AAV G003-20-Australia-2020 VP1, goose parvovirus Yan-1 VP1, or goose parvovirus DY VP1. In an aspect, a disclosed N-terminus of a mammalian VP1 or fragment thereof can comprise the N-terminus of AAV2 VP1, the N-terminus of AAV4 ATCC VR-646 VP1, the N-terminus of AAV5 VP1, the N-terminus of AAV6 VP1, the N-terminus of AAV8 VP1, the N-terminus of AAV9 VP1, or the N-terminus of California sea lion AAV1 1187 VP1.

In an aspect, a disclosed AAV chimeric capsid protein can comprise the sequence set forth in any one of SEQ ID NO:36-SEQ ID NO:40. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:36-SEQ ID NO:40.

In an aspect, a disclosed AAV chimeric capsid protein can comprise the sequence set forth in any one of SEQ ID NO:42-SEQ ID NO:48. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:42-SEQ ID NO:48.

In an aspect, a disclosed AAV chimeric capsid protein can comprise the sequence set forth in any one of SEQ ID NO:50-SEQ ID NO:56. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:50-SEQ ID NO:56.

In an aspect, a disclosed AAV chimeric capsid protein can comprise the sequence set forth in any one of SEQ ID NO:58-SEQ ID NO:64. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:58-SEQ ID NO:64.

In an aspect, a disclosed AAV chimeric capsid protein can comprise the sequence set forth in any one of SEQ ID NO:66-SEQ ID NO:72. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:66-SEQ ID NO:72.

In an aspect, a disclosed AAV chimeric capsid protein can comprise the sequence set forth in any one of SEQ ID NO:74-SEQ ID NO:80. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:74-SEQ ID NO:80.

In an aspect, a disclosed AAV chimeric capsid protein can comprise the sequence set forth in any one of SEQ ID NO:82-SEQ ID NO:88. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:82-SEQ ID NO:88.

In an aspect, a disclosed AAV chimeric capsid protein can comprise a non-mammalian VP2 or fragment thereof. In an aspect, a disclosed non-mammalian VP2 or a fragment thereof can comprise a sauropsidan VP2. In an aspect, a disclosed non-mammalian VP2 or a fragment thereof can comprise a reptilian VP2 or an avian VP2. In an aspect, a disclosed AAV chimeric capsid protein comprises a non-mammalian VP3 or fragment thereof. In an aspect, a disclosed non-mammalian VP2 or a fragment thereof can comprise a sauropsidan VP3. In an aspect, a disclosed non-mammalian VP2 or a fragment thereof can comprise a reptilian VP3 or an avian VP3. In an aspect, a disclosed AAV chimeric capsid protein displays less susceptibility to antibody-mediated neutralization when compared to a disclosed AAV non-chimeric capsid protein. In an aspect, a disclosed AAV chimeric capsid protein can display a higher gene transfer efficiency when compared to a disclosed AAV non-chimeric capsid protein.

In an aspect, a disclosed chimeric capsid protein can be combined with a non-chimeric capsid protein in any combination thereof to, for example, generate a complete AAV capsid.

### 2. AAV Capsids

Disclosed herein is an AAV capsid comprising one or more copies of a disclosed AAV chimeric capsid protein.

Disclosed herein is an AAV capsid comprising one or more copies of a disclosed AAV chimeric capsid protein and one or more copies of a disclosed non-chimeric capsid protein.

Disclosed herein is an AAV capsid comprising 60 VPs, wherein the 60 VPs comprise one or more copies of a disclosed AAV chimeric capsid protein. Disclosed herein is an AAV capsid comprising 60 VPs, wherein the 60 VPs comprise one or more copies of a disclosed AAV chimeric VP1, a disclosed AAV chimeric VP2, a disclosed AAV chimeric VP3, or any combination thereof.

Disclosed herein is an AAV capsid comprising 60 VPs, wherein the 60 VPs comprise one or more copies of a disclosed AAV chimeric VP1, a disclosed AAV chimeric VP2, a disclosed AAV chimeric VP3, or any combination thereof, and one or more copies of a disclosed non-chimeric capsid protein. Disclosed herein is an AAV capsid comprising 60 VPs, wherein the 60 VPs comprise one or more copies of a disclosed AAV chimeric capsid protein, and wherein the AAV capsid evades neutralizing antibodies better than an AAV capsid not having one or more copies of the AAV chimeric capsid protein. Disclosed herein is an AAV capsid comprising 60 VPs, wherein the 60 VPs comprise one or more copies of a disclosed AAV chimeric capsid protein and one or more copies of a disclosed non-chimeric capsid protein, and wherein the AAV capsid evades neutralizing antibodies better than an AAV capsid not having one or more copies of the AAV chimeric capsid protein. Disclosed herein is an AAV capsid comprising 60 VPs, wherein the 60 VPs comprise one or more copies of a disclosed AAV chimeric capsid protein, and wherein the AAV capsid display higher gene transfer efficiency than an AAV capsid not having one or more copies of the AAV chimeric capsid protein. Disclosed herein is an AAV capsid comprising 60 VPs, wherein the 60 VPs comprise one or more copies of a disclosed AAV chimeric capsid protein and one or more copies of a disclosed non-chimeric capsid protein, and wherein the AAV capsid display higher gene transfer efficiency than an AAV capsid not having one or more copies of the AAV chimeric capsid protein. Disclosed herein is an AAV capsid comprising an AAV chimeric capsid protein comprising a non-mammalian VP1 or a fragment thereof; and an N-terminus of a mammalian VP1 or a fragment thereof, wherein the N-terminus of the mammalian VP1 replaces the N-terminus of the non-mammalian VP1.

In an aspect of a disclosed AAV capsid, a disclosed non-mammalian VP1 or a fragment thereof can comprise a sauropsidan VP1. In an aspect, a disclosed non-mammalian VP1 or a fragment thereof can comprise a reptilian VP1. In an aspect, a disclosed non-mammalian VP1 or a fragment thereof can comprise an avian VP1.

In an aspect of a disclosed AAV capsid, a disclosed N-terminus of the non-mammalian VP1 that is replaced by the N-terminus of the mammalian VP1 can comprise about 30 amino acids to about 80 amino acids, about 40 amino acids to about 70 amino acids, or about 50 amino acids to about 60 amino acids.

In an aspect of a disclosed AAV capsid, a disclosed N-terminus of the non-mammalian VP1 that is replaced by the N-terminus of the mammalian VP1 can comprise about 30 amino acids, about 40 amino acids, about 50 amino acids, about 60 amino acids, about 70 amino acids, or about 80 amino acids. In an aspect of a disclosed AAV capsid, a disclosed N-terminus of the mammalian VP1 that replaces the N-terminus of the non-mammalian VP1 can comprise about 30 amino acids to about 80 amino acids, about 40 amino acids to about 70 amino acids, or about 50 amino acids to about 60 amino acids. In an aspect of a disclosed AAV capsid, a disclosed N-terminus of the mammalian VP1 that replaces the N-terminus of the non-mammalian VP1 can comprise about 30 amino acids, about 40 amino acids, about 50 amino acids, about 60 amino acids, about 70 amino acids, or about 80 amino acids.

In an aspect of a disclosed AAV capsid, a disclosed sauropsidan VP1 or fragment thereof can comprise snake AAV VP1, bearded dragon parvovirus 2014 VP1, AAV MHH-05-2015 VP1, duck AAV G003-20-Australia-2020 VP1, goose parvovirus Yan-1 VP1, or goose parvovirus DY VP1. In an aspect of a disclosed AAV capsid, a disclosed N-terminus of a mammalian VP1 or fragment thereof can comprise the N-terminus of AAV2 VP1, the N-terminus of AAV4 ATCC VR-646 VP1, the N-terminus of AAV5 VP1, the N-terminus of AAV6 VP1, the N-terminus of AAV8 VP1, the N-terminus of AAV9 VP1, or the N-terminus of California sea lion AAV1 1187 VP1.

In an aspect of a disclosed AAV capsid, a disclosed AAV chimeric capsid protein can comprise the sequence set forth in any one of SEQ ID NO:36-SEQ ID NO:40, SEQ ID NO:42-SEQ ID NO:48, SEQ ID NO:50-SEQ ID NO:56, SEQ ID NO:58-SEQ ID NO:64, SEQ ID NO: 66-SEQ ID NO:72, SEQ ID NO:74-SEQ ID NO:80, or SEQ ID NO:82-SEQ ID NO:88. In an aspect of a disclosed AAV capsid, a disclosed AAV chimeric capsid protein can comprise a sequence having about 70%, about 75%, about 80%, about 85%, about 90%, or about 95% identity to the sequence set forth in any one of SEQ ID NO:36-SEQ ID NO:40, SEQ ID NO:42-SEQ ID NO: 48, SEQ ID NO:50-SEQ ID NO:56, SEQ ID NO:58-SEQ ID NO:64, SEQ ID NO:66-SEQ ID NO:72, SEQ ID NO:74-SEQ ID NO:80, or SEQ ID NO:82-SEQ ID NO:88. In an aspect of a disclosed AAV capsid, a disclosed AAV chimeric capsid protein can comprise a sequence having about 96%, about 97%, about 98%, or about 99% identity to the sequence set forth in any one of SEQ ID NO:36-SEQ ID NO:40, SEQ ID NO:42-SEQ ID NO:48, SEQ ID NO: 50-SEQ ID NO:56, SEQ ID NO:58-SEQ ID NO:64, SEQ ID NO:66-SEQ ID NO:72, SEQ ID NO:74-SEQ ID NO:80, or SEQ ID NO:82-SEQ ID NO:88.

Disclosed herein is an AAV capsid comprising an AAV chimeric capsid protein comprising a chimeric sauropsidan VP1, wherein the chimeric sauropsidan VP1 comprises an N-terminus of VP1 of a mammalian AAV. In an aspect of a disclosed AAV capsid, a disclosed AAV chimeric capsid protein can comprise the sequence set forth in any one of SEQ ID NO:36-SEQ ID NO: 40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO:74-SEQ ID NO:80, or SEQ ID NO: 82-SEQ ID NO:88. In an aspect of a disclosed AAV capsid, a disclosed AAV chimeric capsid protein can comprise a sequence having about 70%, about 75%, about 80%, about 85%, about 90%, or about 95% identity to the sequence set forth in any one of SEQ ID NO:36-SEQ ID NO: 40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO:74-SEQ ID NO:80, or SEQ ID NO: 82-SEQ ID NO:88. In an aspect of a disclosed AAV capsid, a disclosed AAV chimeric capsid protein can comprise a sequence having about 96%, about 97%, about 98%, or about 99% identity to the sequence set forth in any one of SEQ ID NO:36-SEQ ID NO:40, SEQ ID NO:42-SEQ ID NO:48, SEQ ID NO:50-SEQ ID NO:56, SEQ ID NO:58-SEQ ID NO:64, SEQ ID NO: 66-SEQ ID NO: 72, SEQ ID NO: 74-SEQ ID NO: 80, or SEQ ID NO:82-SEQ ID NO:88.

Disclosed herein is an AAV capsid comprising an AAV chimeric capsid protein comprising the sequence set forth in any one of SEQ ID NO:01-SEQ ID NO:07. Disclosed herein is an AAV capside comprising an AAV chimeric capsid protein comprising a sequence having about 70%, about 75%, about 80%, about 85%, about 90%, or about 95% identity to the sequence set forth in any one of SEQ ID NO:01-SEQ ID NO:07. Disclosed herein is an AAV capsid comprising an AAV chimeric capsid protein comprising a sequence having about 96%, about 97%, about 98%, or about 99% identity to the sequence set forth in any one of SEQ ID NO: 01-SEQ ID NO:07.

Disclosed herein is an AAV capsid comprising an AAV chimeric capsid protein comprising a chimeric VP1/VP2 (partial). In an aspect, a disclosed VP2 (partial) can comprise a primate VP2 (partial). In an aspect, a disclosed chimeric VP1/VP2 (partial) can comprise an AAV1 VP1/VP2 (partial), an AAV2 VP1/VP2 (partial), an AAV8 VP1/VP2 (partial), an AAV9 VP1/VP2 (partial), an AAVhu.37 VP1/VP2 (partial), or an AAVrh. 10 VP1/VP2 (partial). In an aspect, a disclosed chimeric VP1/VP2 (partial) can comprise the sequence set forth in any one of SEQ ID NO:29-SEQ ID NO:34. In an aspect, a disclosed chimeric VP1/VP2 (partial) can comprise a sequence having about 70%, about 75%, about 80%, about 85%, about 90%, or about 95% identity to the sequence set forth in any one of SEQ ID ID NO: 29-SEQ ID NO:34. In an aspect, a disclosed chimeric VP1/VP2 (partial) can comprise a sequence having about 96%, about 97%, about 98%, or about 99% identity to the sequence set forth in any one of SEQ ID ID NO: 29-SEQ ID NO:34.

Disclosed herein is an AAV capsid comprising an AAV chimeric capsid protein comprising a chimeric VP2 (partial)/VP3. In an aspect, a disclosed VP3 can comprise a reptile VP3. In an aspect, a disclosed chimeric VP2 (partial)/VP3 can comprise an avian DA-1 VP2 (partial), a bearded dragon VP2 (partial), a snake VP2 (partial), an avian RSBR151R VP2 (partial), an avian YZ1 VP2 (partial), an avian ZN1 VP2 (partial), an avian BRDF12 VP2 (partial), a Muscovy duck parvovirus FM VP2 (partial), an avian VR865 VP2 (partial), a Muscovy duck MHH 05-2015 VP2 (partial), or a pacific black duck AAV VP2 (partial).

In an aspect of a disclosed AAV capsid, a disclosed chimeric VP2 (partial)/VP3 can comprise the sequence set forth in any one of SEQ ID NO:18-SEQ ID NO:28. In an aspect, a disclosed chimeric VP2 (partial)/VP3 can comprise a sequence having about 70%, about 75%, about 80%, about 85%, about 90%, or about 95% identity to the sequence set forth in any one of SEQ ID ID NO: 18-SEQ ID NO:28. In an aspect, a disclosed chimeric VP2 (partial)/VP3 can comprise a sequence having about 96%, about 96%, about 98%, or about 99% identity to the sequence set forth in any one of SEQ ID ID NO: 18-SEQ ID NO:28.

Disclosed herein is an AAV capsid comprising an AAV chimeric capsid protein comprising a chimeric VP1/VP2 (partial) and a chimeric VP2 (partial)/VP3. In an aspect, a disclosed chimeric VP1/VP2 (partial) can comprise an AAV1 VP1, an AAV2 VP2, an AAV8 VP2, an AAV9 VP, an AAVhu.37 VP1, or an AAVrh.10 VP1, and wherein the chimeric VP2 (partial)/VP3 comprises an avian DA-1 VP2 (partial), a bearded dragon VP2 (partial), a snake VP2 (partial), an avian RSBR151R VP2 (partial), an avian YZ1 VP2 (partial), an avian ZN1 VP2 (partial), an avian BRDF12 VP2 (partial), a Muscovy duck parvovirus FM VP2 (partial), an avian VR865 VP2 (partial), a Muscovy duck MHH 05-2015 VP2 (partial), or a pacific black duck AAV VP2 (partial).

In an aspect, a disclosed chimeric VP1/VP2 (partial) can comprise a primate VP2 (partial), and wherein the chimeric VP2 (partial)/VP3 comprises a reptile VP3. In an aspect, a disclosed chimeric VP1/VP2 (partial) can comprise the sequence set forth in any one of SEQ ID NO: 29-SEQ ID NO:34, and wherein the chimeric VP2 (partial)/VP3 comprises the sequence set forth in any one of SEQ ID NO: 18-SEQ ID NO:28. In an aspect, a disclosed chimeric VP1/VP2 (partial) can comprise a sequence having about 70%, about 75%, about 80%, about 85%, about 90%, or about 95% identity to the sequence set forth in any one of SEQ ID ID NO: 29-SEQ ID NO:34, and wherein the chimeric VP2 (partial)/VP3 comprises a sequence having about 70%, about 75%, about 80%, about 85%, about 90%, or about 95% identity to the sequence set forth in any one of SEQ ID ID NO: 18-SEQ ID NO:28. In an aspect, a disclosed chimeric VP1/VP2 (partial) can comprise a sequence having about 96%, about 96%, about 98%, or about 99% identity to the sequence set forth in any one of SEQ ID ID NO: 29-SEQ ID NO:34, and wherein the chimeric VP2 (partial)/VP3 comprises a sequence having about 96%, about 97%, about 98%, or about 99% identity to the sequence set forth in any one of SEQ ID ID NO: 18-SEQ ID NO:28.

Disclosed herein is an AAV capsid comprising an AAV chimeric capsid protein comprising the sequence set forth in any one of SEQ ID NO:08-SEQ ID NO:17. Disclosed herein is an AAV capsid comprising an AAV chimeric capsid protein comprising a sequence having about 70%, about 75%, about 80%, about 85%, about 90%, or about 95% identity to the sequence set forth in any one of SEQ ID NO:08-SEQ ID NO:17. Disclosed herein is an AAV capsid comprising an AAV chimeric capsid protein comprising a sequence having about 96%, about 97%, about 98%, or about 99% identity to the sequence set forth in any one of SEQ ID NO: 08-SEQ ID NO: 17.

Disclosed herein is an AAV capsid comprising an AAV chimeric capsid protein comprising a chimeric sauropsidan VP1, wherein the chimeric sauropsidan VP1 comprises an N-terminus of a mammalian AAV VP1.

In an aspect, a disclosed chimeric sauropsidan VP1 or fragment thereof can comprise snake AAV VP1, bearded dragon parvovirus 2014 VP1, AAV MHH-05-2015 VP1, duck AAV G003-20-Australia-2020 VP1, goose parvovirus Yan-1 VP1, or goose parvovirus DY VP1.

In an aspect, a disclosed N-terminus of a mammalian VP1 or fragment thereof can comprise the N-terminus of AAV2 VP1, the N-terminus of AAV4 ATCC VR-646 VP1, the N-terminus of AAV5 VP1, the N-terminus of AAV6 VP1, the N-terminus of AAV8 VP1, the N-terminus of AAV9 VP1, or the N-terminus of California sea lion AAV1 1187 VP1.

In an aspect, a disclosed AAV chimeric capsid protein can comprise the sequence set forth in any one of SEQ ID NO:36-SEQ ID NO:40. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:36-SEQ ID NO:40.

In an aspect, a disclosed AAV chimeric capsid protein can comprise the sequence set forth in any one of SEQ ID NO:42-SEQ ID NO:48. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:42-SEQ ID NO:48.

In an aspect, a disclosed AAV chimeric capsid protein can comprise the sequence set forth in any one of SEQ ID NO:50-SEQ ID NO:56. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:50-SEQ ID NO:56.

In an aspect, a disclosed AAV chimeric capsid protein can comprise the sequence set forth in any one of SEQ ID NO:58-SEQ ID NO:64. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:58-SEQ ID NO:64.

In an aspect, a disclosed AAV chimeric capsid protein can comprise the sequence set forth in any one of SEQ ID NO:66-SEQ ID NO:72. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:66-SEQ ID NO:72.

In an aspect, a disclosed AAV chimeric capsid protein can comprise the sequence set forth in any one of SEQ ID NO:74-SEQ ID NO:80. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:74-SEQ ID NO:80.

In an aspect, a disclosed AAV chimeric capsid protein can comprise the sequence set forth in any one of SEQ ID NO:82-SEQ ID NO:88. In an aspect, a disclosed AAV chimeric capsid protein can comprise a sequence having about 70%, about 75%, about 80%, about 85%, about 90%, about 95%, about 96%, about 97%, about 98%, or about 99% identity to the sequence set forth in any one of SEQ ID NO:82-SEQ ID NO:88.

In an aspect of a disclosed AAV capsid, a disclosed AAV chimeric capsid protein can comprise a non-mammalian VP2 or fragment thereof. In an aspect, a disclosed non-mammalian VP2 or a fragment thereof can comprise a sauropsidan VP2. In an aspect, a disclosed non-mammalian VP2 or a fragment thereof can comprise a reptilian VP2 or an avian VP2.

In an aspect of a disclosed AAV capsid, a disclosed AAV chimeric capsid protein comprises a non-mammalian VP3 or fragment thereof. In an aspect, a disclosed non-mammalian VP2 or a fragment thereof can comprise a sauropsidan VP3. In an aspect, a disclosed non-mammalian VP2 or a fragment thereof can comprise a reptilian VP3 or an avian VP3

Disclosed herein is a AAV capsid comprising one or more copies of an AAV chimeric capsid protein comprising the sequence set forth in any one of SEQ ID NO:36-SEQ ID NO:40, SEQ ID NO:42-SEQ ID NO:48, SEQ ID NO:50-SEQ ID NO:56, SEQ ID NO:58-SEQ ID NO: 64, SEQ ID NO:66-SEQ ID NO: 72, SEQ ID NO: 74-SEQ ID NO:80, or SEQ ID NO:82-SEQ ID NO:88. Disclosed herein is a AAV capsid comprising one or more copies of an AAV chimeric capsid protein comprising a sequence having about 70%, about 75%, about 80%, about 85%, about 90%, or about 95% identity to the sequence set forth in any one of SEQ ID NO:36-SEQ ID NO:40, SEQ ID NO:42-SEQ ID NO:48, SEQ ID NO:50-SEQ ID NO:56, SEQ ID NO: 58-SEQ ID NO:64, SEQ ID NO:66-SEQ ID NO:72, SEQ ID NO: 74-SEQ ID NO:80, or SEQ ID NO:82-SEQ ID NO:88. Disclosed herein is a AAV capsid comprising one or more copies of an AAV chimeric capsid protein comprising a sequence having about 96%, about 97%, about 98%, or about 99% identity to the sequence set forth in any one of SEQ ID NO:36-SEQ ID NO: 40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO:74-SEQ ID NO:80, or SEQ ID NO: 82-SEQ ID NO:88.

### 3. Isolated Nucleic Acid Sequences

Disclosed herein is an isolated nucleic acid molecule comprising a nucleic acid sequence encoding an adeno-associated virus (AAV) chimeric capsid protein.

Disclosed herein is an isolated nucleic acid molecule comprising a nucleic acid sequence encoding an adeno-associated virus (AAV) chimeric capsid protein having the sequence set forth in any one of SEQ ID NO:36-SEQ ID NO:40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO: 74-SEQ ID NO:80, or SEQ ID NO:82-SEQ ID NO:88. Disclosed herein is an isolated nucleic acid molecule comprising a nucleic acid sequence encoding an adeno-associated virus (AAV) chimeric capsid protein having a sequence having about 70%, about 75%, about 80%, about 85%, about 90%, or about 95% identity to the sequence set forth in any one of SEQ ID NO: 36-SEQ ID NO:40, SEQ ID NO:42-SEQ ID NO:48, SEQ ID NO:50-SEQ ID NO:56, SEQ ID NO:58-SEQ ID NO:64, SEQ ID NO:66-SEQ ID NO:72, SEQ ID NO:74-SEQ ID NO: 80, or SEQ ID NO:82-SEQ ID NO:88. Disclosed herein is an isolated nucleic acid molecule comprising a nucleic acid sequence encoding an adeno-associated virus (AAV) chimeric capsid protein having a sequence having about 96%, about 97%, about 98%, or about 99% identity to the sequence set forth in any one of SEQ ID NO:36-SEQ ID NO:40, SEQ ID NO:42-SEQ ID NO: 48, SEQ ID NO:50-SEQ ID NO:56, SEQ ID NO:58-SEQ ID NO:64, SEQ ID NO:66-SEQ ID NO:72, SEQ ID NO:74-SEQ ID NO:80, or SEQ ID NO:82-SEQ ID NO:88.

Disclosed herein is an isolated nucleic acid molecule comprising a nucleic acid sequence encoding an adeno-associated virus (AAV) chimeric capsid protein having the sequence set forth in any one of SEQ ID NO:01-SEQ ID NO:07. Disclosed herein is an isolated nucleic acid molecule comprising a nucleic acid sequence encoding an adeno-associated virus (AAV) chimeric capsid protein having a sequence having about 70%, about 75%, about 80%, about 85%, about 90%, or about 95% identity to the sequence set forth in any one of SEQ ID NO:01-SEQ ID NO: 07. Disclosed herein is an isolated nucleic acid molecule comprising a nucleic acid sequence encoding an adeno-associated virus (AAV) chimeric capsid protein having a sequence having about 96%, about 97%, about 98%, or about 99% identity to the sequence set forth in any one of SEQ ID NO:01-SEQ ID NO:07.

Disclosed herein is an isolated nucleic acid molecule comprising a nucleic acid sequence encoding an adeno-associated virus (AAV) chimeric capsid protein having the sequence set forth in any one of SEQ ID NO:08-SEQ ID NO:17. Disclosed herein is an isolated nucleic acid molecule comprising a nucleic acid sequence encoding an adeno-associated virus (AAV) chimeric capsid protein having a sequence having about 70%, about 75%, about 80%, about 85%, about 90%, or about 95% identity to the sequence set forth in any one of SEQ ID NO:08-SEQ ID NO: 17. Disclosed herein is an isolated nucleic acid molecule comprising a nucleic acid sequence encoding an adeno-associated virus (AAV) chimeric capsid protein having a sequence having about 96%, about 97%, about 98%, or about 99% identity to the sequence set forth in any one of SEQ ID NO:08-SEQ ID NO:17.

Disclosed herein is an isolated nucleic acid molecule comprising a nucleic acid sequence encoding an adeno-associated virus (AAV) chimeric capsid protein having the sequence set forth in any one of SEQ ID NO:36-SEQ ID NO:40 or having a sequence having about 70%, about 75%, about 80%, about 85%, about 90%, about 95%, about 96%, about 97%, about 98%, or about 99% identity to the sequence set forth in any one of SEQ ID NO:36-SEQ ID NO:40.

Disclosed herein is an isolated nucleic acid molecule comprising a nucleic acid sequence encoding an adeno-associated virus (AAV) chimeric capsid protein having the sequence set forth in any one of SEQ ID NO:42-SEQ ID NO:48 or having a sequence having about 70%, about 75%, about 80%, about 85%, about 90%, about 95%, about 96%, about 97%, about 98%, or about 99% identity to the sequence set forth in any one of SEQ ID NO:42-SEQ ID NO:48.

Disclosed herein is an isolated nucleic acid molecule comprising a nucleic acid sequence encoding an adeno-associated virus (AAV) chimeric capsid protein having the sequence set forth in any one of SEQ ID NO:50-SEQ ID NO:56 or having a sequence having about 70%, about 75%, about 80%, about 85%, about 90%, about 95%, about 96%, about 97%, about 98%, or about 99% identity to the sequence set forth in any one of SEQ ID NO:50-SEQ ID NO:56.

Disclosed herein is an isolated nucleic acid molecule comprising a nucleic acid sequence encoding an adeno-associated virus (AAV) chimeric capsid protein having the sequence set forth in any one of SEQ ID NO:58-SEQ ID NO:64 or having a sequence having about 70%, about 75%, about 80%, about 85%, about 90%, about 95%, about 96%, about 97%, about 98%, or about 99% identity to the sequence set forth in any one of SEQ ID NO:58-SEQ ID NO:64.

Disclosed herein is an isolated nucleic acid molecule comprising a nucleic acid sequence encoding an adeno-associated virus (AAV) chimeric capsid protein having the sequence set forth in any one of SEQ ID NO:66-SEQ ID NO:72 or having a sequence having about 70%, about 75%, about 80%, about 85%, about 90%, about 95%, about 96%, about 97%, about 98%, or about 99% identity to the sequence set forth in any one of SEQ ID NO:66-SEQ ID NO:72.

Disclosed herein is an isolated nucleic acid molecule comprising a nucleic acid sequence encoding an adeno-associated virus (AAV) chimeric capsid protein having the sequence set forth in any one of SEQ ID NO:74-SEQ ID NO:80 or having a sequence having about 70%, about 75%, about 80%, about 85%, about 90%, about 95%, about 96%, about 97%, about 98%, or about 99% identity to the sequence set forth in any one of SEQ ID NO:74-SEQ ID NO:80.

Disclosed herein is an isolated nucleic acid molecule comprising a nucleic acid sequence encoding an adeno-associated virus (AAV) chimeric capsid protein having the sequence set forth in any one of SEQ ID NO:82-SEQ ID NO:88 or having a sequence having about 70%, about 75%, about 80%, about 85%, about 90%, about 95%, about 96%, about 97%, about 98%, or about 99% identity to the sequence set forth in any one of SEQ ID NO:82-SEQ ID NO:88.

In an aspect, a disclosed nucleic acid sequence can encode a sequence that is less than about 4.5 kilobases.

In an aspect, a disclosed nucleic acid sequence is CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

In an aspect, a disclosed isolated nucleic acid molecule can comprise a single stranded DNA molecule, or a single stranded RNA template, or a double stranded DNA template.

In an aspect, a disclosed isolated nucleic acid molecule can comprise a poly A tail.

### 4. AAV Vectors

Disclosed herein is an AAV vector comprising an AAV capsid comprising 60 VPs, wherein the 60 VPs comprise one or more copies of a disclosed AAV chimeric capsid protein, and wherein the AAV vector evades neutralizing antibodies better than an AAV vector not having one or more copies of the AAV chimeric capsid protein.

Disclosed herein is an AAV vector comprising an AAV capsid comprising 60 VPs, wherein the 60 VPs comprise one or more copies of a disclosed AAV chimeric capsid protein, and wherein the AAV vector can be delivered at a higher titer than an AAV vector not having one or more copies of the AAV chimeric capsid protein.

Disclosed herein is an AAV vector comprising an AAV capsid comprising 60 VPs, wherein the 60 VPs comprise one or more copies of a disclosed AAV chimeric capsid protein, and wherein the AAV demonstrates a higher gene transfer efficiency than an AAV vector not having one or more copies of the AAV chimeric capsid protein.

Disclosed herein is an AAV vector comprising a disclosed AAV capsid and a nucleic acid sequence encoding at least one transgene of interest.

Disclosed herein is an AAV vector comprising an AAV capsid comprising 60 VPs, wherein the 60 VPs comprise one or more copies of a disclosed AAV chimeric capsid protein and one or more copies of a non-chimeric capsid protein, and wherein the AAV vector evades neutralizing antibodies better than an AAV vector not having one or more copies of the AAV chimeric capsid protein.

Disclosed herein is an AAV vector comprising an AAV capsid comprising 60 VPs, wherein the 60 VPs comprise one or more copies of a disclosed AAV chimeric capsid protein and one or more copies of a non-chimeric capsid protein, and wherein the AAV vector can be delivered at a higher titer than an AAV vector not having one or more copies of the AAV chimeric capsid protein.

Disclosed herein is an AAV vector comprising an AAV capsid comprising 60 VPs, wherein the 60 VPs comprise one or more copies of a disclosed AAV chimeric capsid protein and one or more copies of a non-chimeric capsid protein, and wherein the AAV demonstrates a higher gene transfer efficiency than an AAV vector not having one or more copies of the AAV chimeric capsid protein.

Disclosed herein is an AAV vector comprising a disclosed AAV capsid and a nucleic acid sequence encoding at least one transgene of interest.

Disclosed herein is an AAV vector comprising a disclosed AAV chimeric capsid and a nucleic acid sequence encoding at least one transgene of interest.

In an aspect, a disclosed AAV vector can restore one or more aspects of cellular homeostasis and/or cellular functionality and/or metabolic dysregulation. In an aspect, a disclosed vector can restore the functionality and/or structural integrity of a missing, deficient, and/or mutant protein or enzyme.

In an aspect, a therapeutically effective amount of disclosed AAV vector can be delivered via intravenous (IV) administration and can comprise a range of about 1×10¹⁰ vg/kg to about 2×10¹⁴ vg/kg. In an aspect, for example, a disclosed AAV vector can be administered at a dose of about 1×10¹¹ to about 8×10¹³ vg/kg or about 1×10¹² to about 8×10¹³ vg/kg. In an aspect, a disclosed AAV vector can be administered at a dose of about 1×10¹³ to about 6×10¹³ vg/kg. In an aspect, a disclosed AAV vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed AAV vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed AAV vector can be administered at a dose of about 1×10¹² vg/kg. In an aspect, a disclosed AAV vector can be administered at a dose of about 1×10¹¹ vg/kg.

In an aspect, a disclosed AAV vector can be administered in a single dose, or in multiple doses (such as 2, 3, 4, 5, 6, 7, 8, 9 or 10 doses) as needed for the desired therapeutic results. In an aspect, a therapeutically effective amount of disclosed AAV vector can comprise a range determined by a skilled person.

In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV218, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be AAV8. In an aspect, a disclosed AAV vector can be AAVhum.8. In an aspect, a disclosed AAV vector can be a self-complementary AAV as disclosed herein.

In an aspect, a disclosed AAV vector can comprise one or more promoters operably linked to a disclosed transgene. Disclosed promoters are discussed supra while disclosed transgenes are discussed infra. As is known in the art, variation in this distance of a promoter from the transgene can be accommodated without loss of promoter function.

In an aspect, a subject can have or be suspected of having a disease or disorder that can be treated with gene therapy. Examples of such diseases or disorder can include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the I-kappa B dominant mutant, sarcospan, utrophin, mini-utrophin, antisense or RNAi against splice junctions in the dystrophin gene to induce exon skipping (see, e.g., WO 2003/095647), antisense against U7 snRNAs to induce exon skipping (see, e.g., WO 2006/021724), and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [a-galactosidase] and Pompe disease [lysosomal acid α-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., IIC), serca2a, zinc finger proteins that regulate the phospholamban gene, Barkct, P2-adrenergic receptor, p2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like.

Genetic diseases and disorders are discussed supra and include, but are not limited to, diseases and disorders due to a defect in the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1L, ASPM, ASXL1, ASXL2, ASXL3, ATM, ATP7A, ATP7B, ATR, ATRX, BAZIA, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANE1, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNCIH1, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREBIL, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAPIB, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MYO15A, MY018B, MY03A, MYO5A, MYO5B, MY07A, MY09A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKDIL1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAI1, RALGAPA1, RANBP2, RBICC1, RELN, RERE, REV3L, RIC1, RIMS1, RIMS2, RNF213, ROBO1, ROBO2, ROBO3, ROS1, RP1, RPIL1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, S1, SIPAIL3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

Genetic diseases and disorders can also include, but are not limited to, diseases and disorders due to a defect in the following genes: dystrophin including mini- and micro-dystrophins (DMD); titin (TTN); titin cap (TCAP) α-sarcoglycan (SGCA), β-sarcoglycan (SGCB), γ-sarcoglycan (SGCG) or δ-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPD1); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine (N-acetyl)-6-sulfatase (GNS); galactosamine (N-acetyl)-6-sulfatase (GALNS); β-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase α class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel β subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); carnitine palmitoyltransferase 1A (CPTIA); ryanodine receptor 1 (RYR1); ryanodine receptor 2 (RYR2); bridging integrator 1 (BIN1); LARGE xylosyl- and glucuronyltransferase 1 (LARGEI); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic β1 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCH1); notch 3 (NOTCH3); dystrobrevin α (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNAI2); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH: ubiquinone oxidoreductase core subunit 1 (MT-ND1); mitochondrially encoded NADH: ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβR1); transforming growth factor β receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as those disclosed supra) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme (such as those disclosed supra)). In an aspect, restoration can be a partial or incomplete restoration. In an aspect, restoration can be complete or near complete restoration such that the level of expression, activity, and/or functionality is similar to that of a wild-type or control level.

### 5. Pharmaceutical Formulations

Disclosed herein is a pharmaceutical formulation comprising a disclosed AAV chimeric capsid protein or a disclosed AAV chimeric capsid protein or a disclosed AAV chimeric vector.

Disclosed herein is a pharmaceutical formulation comprising an AAV chimeric capsid protein, comprising a non-mammalian VP1 or a fragment thereof; and an N-terminus of a mammalian VP1 or a fragment thereof, wherein the N-terminus of the mammalian VP1 replaces the N-terminus of the non-mammalian VP1. Disclosed herein is a pharmaceutical formulation comprising an AAV chimeric capsid protein, comprising a chimeric sauropsidan VP1, wherein the chimeric sauropsidan VP1 comprises an N-terminus of VP1 of a mammalian AAV. Disclosed herein is a pharmaceutical formulation comprising an AAV chimeric capsid protein comprising the sequence set forth in any one of SEQ ID NO:01-SEQ ID NO:07 or any one of SEQ ID NO:08-SEQ ID NO: 17. Disclosed herein is a pharmaceutical formulation comprising an AAV chimeric capsid protein comprising a chimeric VP1/VP2 (partial). Disclosed herein is a pharmaceutical formulation comprising an AAV chimeric capsid protein comprising a chimeric VP2 (partial)/VP3. Disclosed herein is a pharmaceutical formulation comprising an AAV chimeric capsid protein, comprising a chimeric VP1/VP2 (partial) and a chimeric VP2 (partial)/VP3.

Disclosed herein is a pharmaceutical formulation comprising an AAV chimeric capsid protein comprising a chimeric sauropsidan VP1, wherein the chimeric sauropsidan VP1 comprises an N-terminus of a mammalian AAV VP1.

Disclosed herein is a pharmaceutical formulation comprising an AAV capsid comprising one or more copies of a disclosed AAV chimeric capsid protein. Disclosed herein is a pharmaceutical formulation comprising an AAV capsid comprising one or more copies of a disclosed AAV chimeric capsid protein and one or more copies of a disclosed non-chimeric capsid protein.

Disclosed herein is a pharmaceutical formulation comprising an AAV capsid comprising 60 VPs, wherein the 60 VPs comprise one or more copies of a disclosed AAV chimeric capsid protein. Disclosed herein is a pharmaceutical formulation comprising an AAV capsid comprising 60 VPs, wherein the 60 VPs comprise one or more copies of a disclosed AAV chimeric VP1, a disclosed AAV chimeric VP2, a disclosed AAV chimeric VP3, or any combination thereof. Disclosed herein is a pharmaceutical formulation comprising an AAV capsid comprising 60 VPs, wherein the 60 VPs comprise one or more copies of a disclosed AAV chimeric VP1, a disclosed AAV chimeric VP2, a disclosed AAV chimeric VP3, or any combination thereof, and one or more copies of a disclosed non-chimeric capsid protein. Disclosed herein is a pharmaceutical formulation comprising an AAV capsid comprising 60 VPs, wherein the 60 VPs comprise one or more copies of a disclosed AAV chimeric capsid protein, and wherein the AAV capsid evades neutralizing antibodies better than an AAV capsid not having one or more copies of the AAV chimeric capsid protein. Disclosed herein is a pharmaceutical formulation comprising an AAV capsid comprising 60 VPs, wherein the 60 VPs comprise one or more copies of a disclosed AAV chimeric capsid protein and one or more copies of a disclosed non-chimeric capsid protein, and wherein the AAV capsid evades neutralizing antibodies better than an AAV capsid not having one or more copies of the AAV chimeric capsid protein. Disclosed herein is a pharmaceutical formulation comprising an AAV capsid comprising 60 VPs, wherein the 60 VPs comprise one or more copies of a disclosed AAV chimeric capsid protein, and wherein the AAV capsid display higher gene transfer efficiency than an AAV capsid not having one or more copies of the AAV chimeric capsid protein. Disclosed herein is a pharmaceutical formulation comprising an AAV capsid comprising 60 VPs, wherein the 60 VPs comprise one or more copies of a disclosed AAV chimeric capsid protein and one or more copies of a disclosed non-chimeric capsid protein, and wherein the AAV capsid display higher gene transfer efficiency than an AAV capsid not having one or more copies of the AAV chimeric capsid protein.

Disclosed herein is a pharmaceutical formulation comprising an AAV capsid comprising an AAV chimeric capsid protein comprising a non-mammalian VP1 or a fragment thereof; and an N-terminus of a mammalian VP1 or a fragment thereof, wherein the N-terminus of the mammalian VP1 replaces the N-terminus of the non-mammalian VP1. Disclosed herein is a pharmaceutical formulation comprising an AAV capsid comprising an AAV chimeric capsid protein comprising a non-mammalian VP1 or a fragment thereof; and an N-terminus of a mammalian VP1 or a fragment thereof, wherein the N-terminus of the mammalian VP1 replaces the N-terminus of the non-mammalian VP1.

Disclosed herein is a pharmaceutical formulation comprising an AAV vector comprising an AAV capsid comprising 60 VPs, wherein the 60 VPs comprise one or more copies of a disclosed AAV chimeric capsid protein, and wherein the AAV vector evades neutralizing antibodies better than an AAV vector not having one or more copies of the AAV chimeric capsid protein. Disclosed herein is a pharmaceutical formulation comprising an AAV vector comprising an AAV capsid comprising 60 VPs, wherein the 60 VPs comprise one or more copies of a disclosed AAV chimeric capsid protein, and wherein the AAV vector can be delivered at a higher titer than an AAV vector not having one or more copies of the AAV chimeric capsid protein. Disclosed herein is a pharmaceutical formulation comprising an AAV vector comprising an AAV capsid comprising 60 VPs, wherein the 60 VPs comprise one or more copies of a disclosed AAV chimeric capsid protein, and wherein the AAV demonstrates a higher gene transfer efficiency than an AAV vector not having one or more copies of the AAV chimeric capsid protein. Disclosed herein is an AAV vector comprising a disclosed AAV capsid and a nucleic acid sequence encoding at least one transgene of interest.

Disclosed herein is a pharmaceutical formulation comprising an AAV vector comprising an AAV capsid comprising 60 VPs, wherein the 60 VPs comprise one or more copies of a disclosed AAV chimeric capsid protein and one or more copies of a non-chimeric capsid protein, and wherein the AAV vector evades neutralizing antibodies better than an AAV vector not having one or more copies of the AAV chimeric capsid protein. Disclosed herein is a pharmaceutical formulation comprising an AAV vector comprising an AAV capsid comprising 60 VPs, wherein the 60 VPs comprise one or more copies of a disclosed AAV chimeric capsid protein and one or more copies of a non-chimeric capsid protein, and wherein the AAV vector can be delivered at a higher titer than an AAV vector not having one or more copies of the AAV chimeric capsid protein. Disclosed herein is a pharmaceutical formulation comprising an AAV vector comprising an AAV capsid comprising 60 VPs, wherein the 60 VPs comprise one or more copies of a disclosed AAV chimeric capsid protein and one or more copies of a non-chimeric capsid protein, and wherein the AAV demonstrates a higher gene transfer efficiency than an AAV vector not having one or more copies of the AAV chimeric capsid protein. Disclosed herein is a pharmaceutical formulation comprising an AAV vector comprising a disclosed AAV capsid and a nucleic acid sequence encoding at least one transgene of interest. Disclosed herein is a pharmaceutical formulation comprising an AAV vector comprising a disclosed AAV chimeric capsid and a nucleic acid sequence encoding at least one transgene of interest.

In an aspect, a disclosed formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more immune modulators. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an anaplerotic agent (such as, for example, C7 compounds like triheptanoin or MCT).

In an aspect, a disclosed formulation can comprise an RNA therapeutic. An RNA therapeutic can comprise RNA-mediated interference (RNAi) and/or antisense oligonucleotides (ASO). In an aspect, a disclosed RNA therapeutic can be directed at any protein or enzyme that is overexpressed or is overactive due to a missing, deficient, and/or mutant protein or enzyme. In an aspect, a disclosed RNA therapeutic can comprise therapy delivered via LNPs. In an aspect, a disclosed formulation can comprise an enzyme or enzyme precursor for enzyme replacement therapy (ERT).

In an aspect, a disclosed formulation can comprise a disclosed small molecule. In an aspect, a disclosed small molecule can assist in restoring the functionality and/or structural integrity of a missing, deficient, and/or mutant protein or enzyme (such as, for example, those described supra).

In an aspect, any disclosed pharmaceutical formulation can comprise one or more excipients and/or pharmaceutically acceptable carriers. Excipients and/or pharmaceutically acceptable carriers are known to the art and are discussed supra.

### 6. Plasmids

Disclosed herein are plasmids used in methods of making a disclosed AAV chimeric capsid protein and methods of making a disclosed AAV chimeric capsid. Plasmids and using plasmids are known to the art.

### 7. Cells

Disclosed herein are cells transfected by a disclosed AAV vector. Techniques to achieve transfection and transduction are known to the art and using transfected or transduced cells are known to the art. In an aspect, disclosed herein are human immortalized cells lines transfected by one or more disclosed AAV vectors. In an aspect, disclosed herein are human immortalized cells lines contacted with one or more disclosed pharmaceutical formulations. Disclosed herein are cells obtained from a subject treated with one or more disclosed AAV vectors or one or more disclosed pharmaceutical formulations.

### 8. Animals

Disclosed herein are animals treated with one or more disclosed AAV vectors or one or more disclosed pharmaceutical formulations. Transgenic animals are known to the art as are the techniques to generate transgenic animals. In an aspect, a disclosed transgenic animal does not mount an immune response to treatment with a disclosed AAV vector.

### C. Methods

### 1. Methods of Making

Disclosed herein are methods of making a disclosed AAV chimeric capsid protein and method of making a disclosed AAV chimeric capsid.

In an aspect, a disclosed AAV chimeric capsid can be packaged into virus particles that can be used to deliver the genome for transgene-encoding sequence expression in target cells (e.g., target cells, for example, in a subject in need thereof). In an aspect, a disclosed AAV vector herein can be packaged into particles by transient transfection, use of producer cell lines, combining viral features into Ad-AAV hybrids, use of herpesvirus systems, or production in insect cells using baculoviruses. These methods as well as others are known to the skilled person in the art.

For example, a method of generating a packaging cell for use in a disclosed method can comprise creating a cell line that stably expresses all of the necessary components for AAV particle production. For instance, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes can be introduced into bacterial plasmids by procedures such as GC tailing, addition of synthetic linkers containing restriction endonuclease cleavage sites, or by direct, blunt-end ligation. The packaging cell line can then infected with a helper virus, such as adenovirus. The advantages of this method are that the cells are selectable and are suitable for large-scale production of rAAV. Examples of suitable methods herein employ adenovirus or baculovirus, rather than plasmids, to introduce rAAV genomes and/or rep and cap genes into packaging cells.

### 2. Methods of Treating a Genetic Disease and Disorder

Disclosed herein is a method of treating and/or preventing a genetic disease or disorder, the method comprising administering to a subject in need thereof a therapeutically effective amount of a disclosed AAV vector.

Disclosed herein is a method of treating and/or preventing a genetic disease or disorder, the method comprising administering to a subject in need thereof a therapeutically effective amount of a disclosed AAV vector, wherein the expression of the encoded transgene can restore one or more aspects of cellular homeostasis and/or cellular functionality and/or metabolic dysregulation.

Disclosed herein is a method of treating and/or preventing a genetic disease or disorder, the method comprising administering to a subject in need thereof a therapeutically effective amount of a disclosed AAV vector, wherein expression of the nucleic acid molecule improve gene therapy (e.g., improves mRNA transcript stability and/or improves mRNA transcript translation efficiency).

In an aspect, a therapeutically effective amount of disclosed AAV vector can comprise a range of about 1×10¹⁰ vg/kg to about 2×10¹⁴·vg/kg. In an aspect, for example, a disclosed AAV vector can be administered at a dose of about 1×10¹¹ to about 8×10¹³ vg/kg or about 1×10¹² to about 8×10¹³ vg/kg. In an aspect, a disclosed AAV vector can be administered at a dose of about 1×10¹³ to about 6×10¹³ vg/kg. In an aspect, a disclosed AAV vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed AAV vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed AAV vector can be administered at a dose of about 1×10¹² vg/kg. In an aspect, a disclosed AAV vector can be administered at a dose of about 1×10¹¹ vg/kg. In an aspect, a disclosed AAV vector can be administered in a single dose, or in multiple doses (such as 2, 3, 4, 5, 6, 7, 8, 9 or 10 doses) as needed for the desired therapeutic results. In an aspect, a therapeutically effective amount of disclosed AAV vector can comprise a range determined by a skilled person.

In an aspect of a disclosed method, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are provided below.

| Regulated Variable | Sensor |
|---|---|
| Blood Pressure/Blood | Aortic Body (Aorta), Carotid Body (Carotid |
| Volume/Na⁺ Conc./ | Artery), Atrial Volume Receptors (Heart), |
| | Juxtaglomerular Apparatus (Kidney) |
| Ca²⁺/Mg²⁺/PO₄³⁻ Conc. | Chief Cells (Parathyroid Gland) |
| Glucose | Islet of Langerhans (Pancreas) |
| Osmolarity | Circumventricular Organs (Hypothalamus) |
| pO₂, pCO₂, and pH | Aortic Body (Aorta), Carotid Body (Carotid |
| | Artery), Ventrolateral Medulla (Medulla) |
| Temperature | Thermosensory neurons (Skin), Preoptic |
| | Area (Hypothalamus) |

In an aspect of a disclosed method, contacting a cell can comprising methods known to the art. For example, contacting can comprise administering to a subject one or more disclosed compositions, disclosed pharmaceutical formulations, and/or disclosed vectors.

In an aspect, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed composition, a disclosed pharmaceutical formulation, and/or a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed composition, a disclosed pharmaceutical formulation, and/or a disclosed vector can be administered via LNP administration. In an aspect, a disclosed composition, a disclosed pharmaceutical formulation, and/or a disclosed vector can be concurrently and/or serially administered to a subject via multiple routes of administration. For example, in an aspect, administering a disclosed vector and/or a disclosed pharmaceutical formulation can comprise intravenous administration and intra-cistern magna (ICM) administration. In an aspect, administering a disclosed composition, a disclosed pharmaceutical formulation, and/or a disclosed vector can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect, a disclosed method of treating and/or preventing a genetic disease or disorder can further comprise administering to the subject a therapeutically effective amount of a therapeutic agent. A therapeutic agent can be any disclosed agent that effects a desired clinical outcome.

In an aspect, a disclosed method of treating and/or preventing a genetic disease or disorder can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of treating and/or preventing a genetic disease or disorder can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed vector, a disclosed pharmaceutical formulation, or a combination thereof. In an aspect, a disclosed immune modulator can be administered subcutaneously about 15 minutes before or a few days before a disclosed vector, a disclosed pharmaceutical formulation, or a combination thereof. In an aspect, a disclosed immune modulator can be administered concurrently with a disclosed vector, a disclosed pharmaceutical formulation, or a combination thereof.

In an aspect, a disclosed method of treating and/or preventing a genetic disease or disorder can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of improving transgene stability can further comprise administering one or more proteasome inhibitors or agents that act on plasma cells prior to administering a disclosed vector or a disclosed pharmaceutical formulation. In an aspect, a disclosed method can comprise administering one or more proteasome inhibitors or one or more agents that act on plasma cells concurrently with administering a disclosed vector or a disclosed pharmaceutical formulation. In an aspect, a disclosed method can comprise administering one or more proteasome inhibitors or one or more agents that act on plasma cells subsequent to administering a disclosed vector or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of treating and/or preventing a genetic disease or disorder can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of treating and/or preventing a genetic disease or disorder can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method can further comprise administering lipid nanoparticles (LNPs). In an aspect, LNPs can be organ-targeted. In an aspect, LNPs can be liver-targeted or testes-targeted. For example, in an aspect, mRNA therapy with LNP encapsulation for systemic delivery to a subject has the potential to restore the functionality and/or structural integrity of a missing, deficient, and/or mutant protein or enzyme.

In an aspect, a disclosed method of treating and/or preventing a genetic disease or disorder can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step one or more times such as, for example, repeating the administering of a disclosed isolated nucleic acid molecule, a disclosed AAV chimeric capsid protein, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a method can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, a disclosed method of treating and/or preventing a genetic disease or disorder can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein). In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

### D. Kits

Disclosed herein is a kit comprising a disclosed AAV chimeric capsid protein, a disclosed vector, a disclosed pharmaceutical formulation, or any combination thereof. Disclosed herein is a kit comprising one or more disclosed AAV chimeric capsid protein, disclosed vectors, one or more disclosed pharmaceutical formulations, or any combination thereof. In an aspect, a kit can comprise a disclosed vector, a disclosed pharmaceutical formulation, a disclosed RNA therapeutic, or a combination thereof, and one or more agents. “Agents” and “Therapeutic Agents” are known to the art and are described supra.

In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to a missing, deficient, and/or mutant protein or enzyme (such as, for example, those affected by a disclosed genetic disease or disorder).

In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed vector, a disclosed pharmaceutical formulation, a disclosed RNA therapeutic, or a combination thereof, and a label or package insert with instructions for use. In an aspect, suitable containers include, for example, bottles, vials, syringes, blister pack, etc. The containers can be formed from a variety of materials such as glass or plastic. The container can comprise a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed vector, a disclosed pharmaceutical formulation, a disclosed RNA therapeutic, or a combination thereof can be used for treating, preventing, inhibiting, and/or ameliorating a disease or disorder or complications and/or symptoms associated with a disease or disorder. A kit can comprise additional components necessary for administration such as, for example, other buffers, diluents, filters, needles, and syringes.

In an aspect, a disclosed kit can be used in a disclosed method such as, for example, a disclosed method of treating and/or preventing a genetic disease or disorder or a method of improving gene therapy.

### E. Miscellaneous

In some aspects, AAV vectors herein may be engineered to generate one or more chimera capsid proteins. As used herein, a “chimera” protein refers to a protein created through the joining of two or more genes that originally coded for separate proteins. In some aspects, chimera capsid proteins herein may be a protein created from the fusion of two proteins that originated from two genes that originally coded for separate proteins.

In some aspects, chimera capsid proteins herein may be created from the fusion of two proteins having a different species of origin. In some aspects, at least one polynucleotide encoding for a chimera capsid protein herein may be a primate. In some aspects, at least one polynucleotide herein may encode for a chimera capsid protein herein having an amino acid sequence having about 85% (e.g., about 85%, 90%, 95%, 99%, 100%) similarity to a naturally occurring primate capsid protein. In some aspects, at least one polynucleotide encoding for a chimera capsid protein herein may be a human or a non-human primate. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans.

In some aspects, at least one polynucleotide encoding for a chimera capsid protein herein may be VP1 or a fragment of VP1, wherein the VP1 is primate in origin. In some aspects, at least one polynucleotide encoding for a chimera capsid protein herein may be VP2 or a fragment of VP2, wherein the VP2 is primate in origin. In some aspects, at least one polynucleotide encoding for a chimera capsid protein herein may be VP3 or a fragment of VP3, wherein the VP3 is primate in origin.

In some aspects, at least one polynucleotide encoding for a chimera capsid protein herein may be a primate capsid protein of any of AAV1, AAV2, AAV3, AAV4, AAV5, AAV6, AAV7, AAV8, or AAV9. In some aspects, at least one polynucleotide encoding for a chimera capsid protein herein may be a primate VP1 or fragment thereof of any of AAV1, AAV2, AAV3, AAV4, AAV5, AAV6, AAV7, AAV8, or AAV9. In some aspects, at least one polynucleotide encoding for a chimera capsid protein herein may be a primate VP2 or fragment thereof of any of AAV1, AAV2, AAV3, AAV4, AAV5, AAV6, AAV7, AAV8, or AAV9. In some aspects, at least one polynucleotide encoding for a chimera capsid protein herein may be a primate VP3 or fragment thereof of any of AAV1, AAV2, AAV3, AAV4, AAV5, AAV6, AAV7, AAV8, or AAV9.

In some aspects, at least one polynucleotide encoding for a chimera capsid protein herein may be a reptile. “Reptile” as used herein includes animals of the class Sauropsida including animals in the class Aves (birds or “avian”) and the orders Squamata (e.g., lizards, bearded dragons, snakes, amphisbaenids (worm-lizards)), Chelonia or Testudines (e.g., turtles) Crocodilia (e.g., crocodiles, caimans, alligators), and Rhynchocephalia (e.g., tuatars from New Zealand). In some aspects, at least one polynucleotide herein may encode for a chimera capsid protein having an amino acid sequence of about 85% (e.g., about 85%, 90%, 95%, 99%, 100%) similarity to a naturally occurring reptile capsid protein.

In some aspects, at least one polynucleotide encoding for a chimera capsid protein herein may be VP1 or a fragment of VP1 wherein the VP1 is reptile in origin. In some aspects, at least one polynucleotide encoding for a chimera capsid protein herein may be VP2 or a fragment of VP2 wherein the VP2 is reptile in origin. In some aspects, at least one polynucleotide encoding for a chimera capsid protein herein may be VP3 or a fragment of VP3 wherein the VP3 is reptile in origin.

In some aspects, at least one polynucleotide encoding for a chimera capsid protein herein may be from an avian adeno-associated virus strain. In some aspects, at least one polynucleotide may encode for a chimera capsid protein herein having an amino acid sequence sharing about 85% (e.g., about 85%, 90%, 95%, 99%, 100%) similarity to a naturally occurring avian capsid protein. In some aspects, at least one polynucleotide may encode for a chimera capsid protein herein having an amino acid sequence sharing about 85% (e.g., about 85%, 90%, 95%, 99%, 100%) similarity to SEQ ID NO:35.

In some aspects, at least one polynucleotide encoding for a chimera capsid protein herein may be from a snake adeno-associated virus strain. In some aspects, at least one polynucleotide may encode for a chimera capsid protein herein having an amino acid sequence sharing about 85% (e.g., about 85%, 90%, 95%, 99%, 100%) similarity to a naturally occurring snake capsid protein. In some aspects, at least one polynucleotide may encode for a chimera capsid protein herein having an amino acid sequence sharing about 85% (e.g., about 85%, 90%, 95%, 99%, 100%) similarity to SEQ ID NO:41.

In some aspects, at least one polynucleotide encoding for a chimera capsid protein herein may be from a bearded dragon adeno-associated virus strain. In some aspects, at least one polynucleotide may encode for a chimera capsid protein herein having an amino acid sequence sharing about 85% (e.g., about 85%, 90%, 95%, 99%, 100%) similarity to a naturally occurring bearded dragon capsid protein. In some aspects, at least one polynucleotide may encode for a chimera capsid protein herein having an amino acid sequence sharing about 85% (e.g., about 85%, 90%, 95%, 99%, 100%) similarity to SEQ ID NO:49.

In some aspects, one polynucleotide encoding for a chimera capsid protein herein may be a primate and a second polynucleotide encoding the chimera capsid protein may be a reptile. In some aspects, polynucleotides herein may encode for a chimera capsid protein that is a fusion of a primate capsid protein and a reptile capsid protein. In some aspects, polynucleotides herein may encode for a chimera capsid protein that is a fusion of a primate VP1 or fragment thereof, a primate VP2 or fragment thereof, and/or a primate VP3 or fragment thereof, and a reptile capsid protein. In some aspects, polynucleotides herein may encode for a chimera capsid protein that is a fusion of a reptile VP1 or fragment thereof, a reptile VP2 or fragment thereof, and/or a reptile VP3 or fragment thereof, and a primate capsid protein. In some aspects, polynucleotides herein may encode for a chimera capsid protein that is a fusion of a primate VP1 or fragment thereof, a primate VP2 or fragment thereof, and/or a primate VP3 or fragment thereof, with a reptile VP1 or fragment thereof, a reptile VP2 or fragment thereof, and/or a reptile VP3 or fragment thereof. In some aspects, polynucleotides herein may encode for a chimera capsid protein comprising a primate VP1, a primate VP2 fragment, a reptile VP2 fragment, and a reptile VP3. In some aspects, polynucleotides herein may encode for a chimera capsid protein comprising a primate VP1, a primate VP2 fragment, a reptile VP2 fragment, and a reptile VP3 wherein the primate VP1 and primate VP2 fragment may be any of AAV1, AAV2, AAV3, AAV4, AAV5, AAV6, AAV7, AAV8, or AAV9.

In some aspects, chimera capsid proteins herein may include a primate VP1 and a primate VP2 fragment. In some aspects, chimera capsid proteins herein may include a primate VP1 and a primate VP2 fragment that share at least about 85% (e.g., about 85%, 90%, 95%, 99%, or 100%) amino acid sequence similarity with any one of the sequences set forth in SEQ ID NOs: 27-32. In some aspects, chimera capsid proteins herein may include a reptile VP3 and a reptile VP2 fragment. In some aspects, chimera capsid proteins herein may include a reptile VP3 and a reptile VP2 fragment that share at least about 85% (e.g., about 85%, 90%, 95%, 99%, or 100%) amino acid sequence similarity with any one of the sequences set forth in SEQ ID NOs: 14-26.

In some aspects, chimera capsid proteins herein may include a primate VP1, a fragment primate VP2, a fragment reptile VP2, and a reptile VP3. In some aspects, chimera capsid proteins herein may include a primate VP1, a fragment primate VP2 that share at least about 85% (e.g., about 85%, 90%, 95%, 99%, or 100%) amino acid sequence similarity with any one of the sequences set forth in SEQ ID NO:29-SEQ ID NO:34 and a fragment reptile VP2, and a reptile VP3 that share at least about 85% (e.g., about 85%, 90%, 95%, 99%, or 100%) amino acid sequence similarity with any one of the sequences set forth in SEQ ID NO:18-SEQ ID NO:28.

In some aspects, chimera capsid proteins herein may share at least about 85% (e.g., about 85%, 90%, 95%, 99%, or 100%) amino acid sequence similarity with any one of the sequences set forth in SEQ ID NO:01-SEQ ID NO:03 or SEQ ID NO:08-SEQ ID NO:17. In accordance with some aspects herein, chimera capsid proteins herein may have any one of the sequences set forth in SEQ ID NO:01-SEQ ID NO:03 or SEQ ID NO:08-SEQ ID NO:17.

In certain aspects, the present disclosure provides AAV vectors comprising one or more of the chimera capsid proteins disclosed herein. As used herein, a “vector” refers to any molecule or moiety which transports, transduces, or otherwise acts as a carrier of a heterologous molecule. A “viral vector” is a vector which comprises one or more polynucleotide regions encoding or comprising a payload molecule of interest, e.g., a transgene, a polynucleotide encoding a polypeptide or multi-polypeptide or a modulatory nucleic acid. Viral vectors of the present invention may be produced recombinantly using methods known in the art. Such techniques are explained fully in the literature, such as in Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; and Cell Biology: A Laboratory Notebook (J. E. Cellis, ed., 1989) Academic Press.

In certain aspects, AAV viral particles disclosed herein can have an AAV vector for expressing one or more of the chimera capsid proteins disclosed herein. An AAV vector is derived from the wild type genome of a virus, such as AAV, by using molecular methods to remove the wild type genome from the virus (e.g., AAV), and replacing with a non-native nucleic acid, such as a heterologous polynucleotide sequence (e.g., a coding sequence for a transgene of interest). Typically, for AAV vectors, one or both inverted terminal repeat (ITR) sequences of the wild type AAV genome are retained in the AAV vector whereas other parts of the wild type viral genome are replaced with a non-native sequence such as a heterologous polynucleotide sequence between the retained ITRs. The AAV vectors disclosed herein can encompass AAV genome-derived backbone elements, a coding sequence for a chimera capsid protein disclosed herein, and a suitable promoter in operable linkage to the coding sequence. In some examples, AAV vectors disclosed herein can further comprise regulatory sequences regulating expression and/or secretion of the encoded protein. Examples include, but are not limited to, enhancers, polyadenylation signal sites, internal ribosome entry sites (IRES), sequences encoding protein transduction domains (PTD), microRNA-target sites, or a combination thereof.

In some examples, AAV vectors herein may be a regular AAV vector comprising a single stranded nucleic acid. In other examples, AAV vectors disclosed herein may be a self-complementary AAV vector capable of comprising double stranded portions therein.

In some aspects, AAV vectors disclosed herein may have one or more AAV-genome derived backbone elements, which refer to the minimum AAV genome elements required for the bioactivity of the AAV vectors. For example, the AAV-genome derived backbone elements may include the packaging site for the AAV vector to be assembled into an AAV viral particle, one or more of the chimera capsid proteins disclosed herein, elements needed for vector replication, and/or expression of a transgene-encoding sequence comprised therein in host cells.

In some examples, AAV vector backbones disclosed herein may include at least one inverted terminal repeat (ITR) sequence. In some examples, AAV vector backbones herein may include two ITR sequences. In some examples, one ITR sequence can be 5′ of a polynucleotide sequence coding for a transgene. In some examples, one ITR sequence can be 3′ of a polynucleotide sequence coding for a transgene. In some examples, a polynucleotide sequence coding for a transgene herein can be flanked on either side by an ITR sequence.

In some aspects, AAV vectors herein may include sequences or components originating from at least one distinct AAV serotype. In some examples, AAV vector backbones disclosed herein may include at least ITR sequence from one distinct AAV serotype. In some examples, AAV vector backbones disclosed herein may include at least ITR sequence from one distinct human AAV serotype. Such a human AAV may be derived from any known serotype, e.g., from any one of serotypes 1-11. In some examples, AAV serotypes used herein have a tropism for the central nervous system (CNS), cardiac tissues, skeletal muscle, and/or liver tissues. In some examples, AAV vector backbones disclosed herein may have an ITR sequence of serotype AAV2, AAV8, or AAV9.

In some aspects, AAV vectors herein can be a pseudotyped AAV vector, (i.e., comprises sequences or components originating from at least two distinct AAV serotypes). In some aspects, a pseudotyped AAV vector herein may include an AAV genome backbone derived from one AAV serotype, and a capsid protein derived at least in part from a distinct AAV serotype. In some examples, pseudotyped AAV vectors herein can have an AAV2 genome backbone and a capsid protein derived from an AAV serotype having a tropism for heart tissue (e.g., AAV1, AAV2, AAV4, AAV5, AAV8, or AAV9). Specific examples of such pseudotyped AAV vectors include, without limitation, vectors comprising an AAV2-derived genome in an AAV5-derived capsid; or vectors comprising an AAV2-derived genome in an AAV8-derived capsid; or vectors comprising an AAV2-derived genome in an AAV9-derived capsid or vectors comprising an AAV2-derived genome in an AAV1-derived capsid.

In order to analyze the success of viral vector-mediated gene transfer, it may be important to be able to monitor both the distribution of the vector and the effectiveness of vector-mediated gene expression. This can be achieved by subcloning a reporter gene into the viral vector backbone. In some examples, AAV vector backbones disclosed herein may contain a reporter gene. Several reporter genes are commonly used for this purpose and include, but are not limited to, fluorescent proteins of various colors (including green fluorescent protein (GFP), red fluorescent protein (RFP)), E. coli β-galactosidase (LacZ), and various forms of luciferase (Luc). In some examples, AAV vector backbones disclosed herein may contain GFP.

The vector constructs disclosed herein may be prepared using known techniques. (See e.g., Current Protocols in Molecular Biology, Ausubel., F. et al., eds, Wiley and Sons, New York 1995). Fragment length can be chosen so that the recombinant genome does not exceed the packaging capacity of the AAV particle. If necessary, a “stuffer” DNA sequence can be added to the construct to maintain standard AAV genome size for comparative purposes. Such a fragment may be derived from such non-viral sources, e.g., lacZ, or other genes which are known and available to those skilled in the art.

In some aspects, AAV vectors disclosed herein can be self-complementary AAV (scAAV) vectors. Self-complementary AAV (scAAV) vectors contain complementary sequences that are capable of spontaneously annealing (folding back on itself to form a double-stranded genome) when entering into infected cells, thus circumventing the need for converting a single-stranded DNA vector using the cell's DNA replication machinery. An AAV herein having a self-complementing genome can quickly form a double stranded DNA molecule by virtue of its partially complementing sequences (e.g., complementing coding and non-coding strands of a transgene-encoding sequence).

In some aspects, a scAAV viral vector disclosed herein may comprise a first heterologous polynucleotide sequence and a second heterologous polynucleotide sequence, which can form intrastrand base pairs. In some examples, the first heterologous polynucleotide sequence and the second heterologous polynucleotide sequence are linked by a sequence that facilitates intrastrand base pairing; e.g., to form a hairpin DNA structure. In some examples, the dimeric structure of a scAAV vector upon entering a cell can be stabilized by a mutation or a deletion of one of the two terminal resolution sites (trs). As trs are Rep-binding sites contained within each ITR, a mutation or a deletion of such trs may prevent cleavage of a dimeric structure of a scAAV vector by AAV Rep proteins to form monomers. In some aspects, a scAAV viral vector disclosed herein may include a truncated 5′ inverted terminal repeats (ITR), a truncated 3′ ITR, or both. In some examples, a scAAV vector disclosed herein may comprise a truncated 3′ ITR, in which the D region or a portion thereof (e.g., the terminal resolution sequence therein) may be deleted. Such a truncated 3′ ITR may be located between the first heterologous polynucleotide sequence and a second heterologous polynucleotide sequence noted above.

### (3) Promoters

In some aspects, AAV vectors disclosed herein comprise further elements necessary for expression, such as at least one suitable promoter which controls the expression of the transgene-encoding sequence. Such a promoter may be ubiquitous, tissue-specific, strong, weak, regulated, chimeric, etc., to allow efficient and suitable production of the protein in the infected tissue. The promoter may be homologous to the encoded protein, or heterologous, including cellular, viral, fungal, plant or synthetic promoters. Most preferred promoters for use herein may be functional in human cells. Non-limiting examples of ubiquitous promoters include viral promoters, particularly the CMV promoter, the RSV promoter, the SV40 promoter, etc. and cellular promoters such as the PGK (phosphoglycerate kinase) promoter. In some aspects, viral promoters herein can be a CMV promoter, a SV40 promoter, or any combination thereof.

In some aspects, AAV vectors disclosed herein may comprise further elements necessary for expression, such as at least one suitable promoter which controls the expression of the transgene-encoding sequence after infection of the appropriate cells. Suitable promoters for use herein include, in addition to the AAV promoters, e.g. the cytomegalovirus (CMV) promoter or the chicken beta actin/cytomegalovirus hybrid promoter (CAG), an endothelial cell-specific promoter such as the VE-cadherin promoter, as well as steroid promoters and metallothionein promoters. In some aspects, the promoter used in the vectors disclosed herein can be a CAG promoter.

In some aspects, the transgene-encoding sequence according to the invention comprises a tissue specific promoter which is functionally linked to the transgene-encoding sequence to be expressed. Accordingly, the specificity of the vectors according to the disclosure for the tissue (e.g., brain, heart, muscle, liver) can be further increased. In some examples, a vector disclosed herein can have a tissue-specific promoter whose activity in the specific tissue is at least about 2-fold, 5-fold, 10-fold, 20-fold, 50-fold or 100-fold higher than in a tissue which is not the specific tissue. In some examples, a tissue specific promoter herein is a human a tissue specific promoter. In some examples, the expression cassette can also include an enhancer element for increasing the expression levels of exogenous protein to be expressed. Furthermore, the expression cassette may further comprise polyadenylation sequences, such as the SV40 polyadenylation sequences or polyadenylation sequences of bovine growth hormone.

In some aspects, AAV vectors disclosed herein may include one or more conventional control elements which are operably linked to the transgene-encoding sequence in a manner which permits its transcription, translation and/or expression in a cell transfected with the plasmid vector or infected with the virus produced by the invention. As used herein, “operably linked” sequences may include both expression control sequences that are contiguous with the transgene-encoding sequence and expression control sequences that act in trans or at a distance to control the transgene-encoding sequence. Expression control sequences may further comprise appropriate transcription initiation, termination, promoter and enhancer sequences; efficient RNA processing signals such as splicing and polyadenylation (poly A) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized herein.

In some aspects, an AAV vector disclosed herein may include a modified capsid, including proteins or peptides of non-viral origin or structurally modified, to alter the tropism of the vector. For example, the capsid may include a ligand of a particular receptor, or a receptor of a particular ligand, to target the vector towards cell type(s) expressing said receptor or ligand, respectively.

Disclosed herein is an adeno-associated virus (AAV) chimera capsid protein comprising three viral proteins (VP1, VP2, or VP3) wherein at least one VP or a fragment thereof shares at least 85% amino acid sequence similarity with a non-mammalian VP and at least one other VP or a fragment thereof shares at least 85% nucleic acid sequence similarity with a mammalian VP.

In an aspect, a disclosed chimera capsid protein can comprise a VP1 sharing at least 85% amino acid sequence similarity with a mammalian VP. In an aspect, a disclosed chimera capsid protein can comprise a VP3 sharing at least 85% amino acid sequence similarity with a non-mammalian VP. In an aspect, a disclosed chimera capsid protein can comprise a VP1 and a fragment of VP2 that share at least 85% amino acid sequence similarity with a mammalian VP.

In an aspect, a disclosed chimera capsid protein can comprise the VP1 and the fragment of VP2 that share at least 85% amino acid sequence similarity with a mammalian VP comprised of any one of the sequences set forth in SEQ ID NO:29-SEQ ID NO:34.

In an aspect, a disclosed chimera capsid protein can comprise a VP3 and a fragment of VP2 that share at least 85% amino acid sequence similarity with a non-mammalian VP. In an aspect, a disclosed chimera capsid protein can comprise the VP3 and the fragment of VP2 that share at least 85% amino acid sequence similarity with a non-mammalian VP comprised of any one of the sequences set forth in SEQ ID NO:18-SEQ ID NO:28. In an aspect, a disclosed chimera capsid protein can comprises a VP1 and a fragment of VP2 comprising any one of the sequences set forth in SEQ ID NO:29-SEQ ID NO:34; and a VP3 and a fragment of VP2 comprising any one of the sequences set forth in SEQ ID NO:18-SEQ ID NO:28. In an aspect, a disclosed chimera capsid protein can comprise any one of the sequences set forth in SEQ ID NO: 08-SEQ ID NO:17.

In an aspect, a disclosed chimera capsid protein can comprise an amino acid sequence from VPs derived from one or more AAV serotypes. In an aspect, one or more disclosed AAV serotypes can comprise AAV2, AAV8, AAV9, or any combination thereof.

Disclosed herein is an AAV capsid comprised of 60 VP copies derived from a disclosed chimera capsid protein or any combination thereof. Disclosed herein is an AAV vector comprising a disclosed AAV capsid, wherein the disclosed AAV capsid comprising a polynucleotide encoding at least one transgene of interest.

Disclosed herein is a AAV vector comprising a disclosed AAV chimera capsid protein can display less susceptibility to antibody-mediated neutralization compared to an AAV vector derived from a mammalian AAV capsid protein alone. In an aspect, a disclosed AAV vector comprising AAV chimera capsid proteins can be produced at a higher titer compared to an AAV vector comprising non-mammalian AAV VPs alone. In an aspect, a disclosed AAV vector comprising AAV chimera capsid proteins can display higher gene transfer efficiency compared to an AAV vector comprising non-mammalian AAV VPs alone.

Disclosed herein is an adeno-associated virus (AAV) particle comprising a disclosed AAV chimera capsid protein. In an aspect, a disclosed AAV particle can further comprise a polynucleotide encoding at least one transgene of interest.

Disclosed herein is a pharmaceutical composition comprising a disclosed AAV vector and at least one pharmaceutically acceptable carrier.

Disclosed herein is a method of introducing a gene into a target cell, the method comprising contacting a target cell with a disclosed AAV vector, a disclosed AAV particle, or a disclosed pharmaceutical composition. Disclosed a method of delivering a transgene to a target cell in a subject, the method comprising administering to the subject a disclosed AAV vector, a disclosed AAV particle, or a disclosed pharmaceutical composition. In an aspect of a disclosed AAV vector, a disclosed AAV particle or a disclosed pharmaceutical composition is administered to the subject following at least one prior dosage with an AAV vector composed of mammalian AAV VPs alone.

In some aspects, AAV vectors disclosed herein may be prepared or derived from various serotypes of AAVs. The term “serotype” is a distinction with respect to an AAV having a capsid which is serologically distinct from other AAV serotypes. Serologic distinctiveness is determined on the basis of the lack of cross-reactivity between antibodies to the AAV as compared to other AAV. Cross-reactivity can be measured using methods known in the art. For example, cross-reactivity herein may be measured using a neutralizing antibody assay. For this assay polyclonal serum is generated against a specific AAV in a rabbit or other suitable animal model using the adeno-associated viruses. In this assay, the serum generated against a specific AAV is then tested in its ability to neutralize either the same (homologous) or a heterologous AAV. The dilution that achieves 50% neutralization is considered the neutralizing antibody titer. If for two AAVs the quotient of the heterologous titer divided by the homologous titer is lower than 16 in a reciprocal manner, those two vectors are considered as the same serotype. Conversely, if the ratio of the heterologous titer over the homologous titer is 16 or more in a reciprocal manner the two AAVs are considered distinct serotypes.

In some aspects, AAV vectors herein may be mixed of at least two serotypes of AAVs or with other types of viruses to produce chimeric (e.g., pseudotyped) AAV viruses. In some aspects, AAV vectors herein may be a human serotype AAV vector. Such a human AAV may be derived from any known serotype, e.g., from any one of serotypes 1-11.

In some aspects, AAV vectors herein may be packaged into virus particles which can be used to deliver the genome for transgene-encoding sequence expression in target cells. In some aspects, AAV vectors disclosed herein can be packaged into particles by transient transfection, use of producer cell lines, combining viral features into Ad-AAV hybrids, use of herpesvirus systems, or production in insect cells using baculoviruses.

A method of generating a packaging cell for use herein can involve creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing, addition of synthetic linkers containing restriction endonuclease cleavage sites, or by direct, blunt-end ligation. The packaging cell line is then infected with a helper virus, such as adenovirus. The advantages of this method are that the cells are selectable and are suitable for large-scale production of rAAV. Examples of suitable methods herein employ adenovirus or baculovirus, rather than plasmids, to introduce rAAV genomes and/or rep and cap genes into packaging cells.

In some aspects, AAV vectors and/or AAV particles herein may have one or more improvements compared to naturally isolated AAV vectors. As used herein, a “naturally isolated AAV vector” refers to a vector that does not comprise one or more of the chimera capsid proteins disclosed herein. In some aspects, AAV vectors and/or AAV particles herein may have increased gene transfer efficiency in a cell compared to naturally isolated AAV vectors. In some aspects, AAV vectors and/or AAV particles herein may have at least about 2-fold to about 50-fold (e.g., about 2-, 4-, 6-, 8-, 10-, 20-, 30-, 40-, 50-fold) increased gene transfer efficiency in a cell compared to naturally isolated AAV vectors.

In some aspects, AAV vectors and/or AAV particles herein may have a higher vector titer compared to naturally isolated AAV vectors. In some aspects, AAV vectors and/or AAV particles herein may have at least about 2-fold to about 50-fold (e.g., about 2-, 4-, 6-, 8-, 10-, 20-, 30-, 40-, 50-fold) higher vector titer compared to naturally isolated AAV vectors.

In some aspects, AAV vectors and/or AAV particles herein may be less susceptible to antibody-mediated neutralization compared to naturally isolated AAV vectors. In some aspects, AAV vectors and/or AAV particles herein may be less susceptible to antibody-mediated neutralization by about 2-fold to about 50-fold (e.g., about 2-, 4-, 6-, 8-, 10-, 20-, 30-, 40-, 50-fold) compared to naturally isolated AAV vectors. In some aspects, AAV vectors and/or AAV particles herein may be less susceptible to antibody-mediated neutralization for at least about 1 hour to about 24 hours (e.g., about 1, 2, 4, 8, 12, 16, 20, 24 hours) after administration to a subject compared to naturally isolated AAV vectors.

In some aspects, AAV vectors and/or AAV particles herein may produce lower levels of anti-AAV antibodies after at least one administration to a subject herein compared to naturally isolated AAV vectors. In some aspects, AAV vectors and/or AAV particles herein may produce about 2-fold to about 50-fold (e.g., about 2-, 4-, 6-, 8-, 10-, 20-, 30-, 40-, 50-fold) less anti-AAV antibodies after at least one administration to a subject herein compared to naturally isolated AAV vectors. In some aspects, gene therapies comprising AAV vectors and/or AAV particles herein can be administered about 2 times to about 10 times (e.g., about 2, 3, 4, 5, 6, 7, 8, 9, 10) to a subject herein without becoming susceptible to antibody-mediated neutralization.

In some aspects, any of the AAV vectors and/or AAV viral particles disclosed herein may be formulated to form a pharmaceutical composition. In some examples, pharmaceutical composition herein can further include a pharmaceutically acceptable carrier, diluent or excipient. Any of the pharmaceutical compositions to be used in the present methods can comprise pharmaceutically acceptable carriers, excipients, or stabilizers in the form of lyophilized formations or aqueous solutions.

The carrier in the pharmaceutical composition must be “acceptable” in the sense that it is compatible with the active ingredient of the composition, and preferably, capable of stabilizing the active ingredient and not deleterious to the subject to be treated. For example, “pharmaceutically acceptable” may refer to molecular entities and other ingredients of compositions comprising such that are physiologically tolerable and do not typically produce untoward reactions when administered to a mammal (e.g., a human). In some examples, the “pharmaceutically acceptable” carrier used in the pharmaceutical compositions disclosed herein may be those approved by a regulatory agency of the Federal or a state government or listed in the U.S. Pharmacopeia or other generally recognized pharmacopeia for use in mammals, and more particularly in humans.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20^{th} Ed. (2000) Lippincott Williams and Wilkins, Ed. K. E. Hoover.

In some aspects, the pharmaceutical compositions or formulations are for parenteral administration, such as intravenous, intracerebroventricular injection, intra-cisterna magna injection, intra-parenchymal injection, or a combination thereof. Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. Pharmaceutical compositions disclosed herein may further comprise additional ingredients, for example preservatives, buffers, tonicity agents, antioxidants and stabilizers, nonionic wetting or clarifying agents, viscosity-increasing agents, and the like. The pharmaceutical compositions described herein can be packaged in single unit dosages or in multi-dosage forms.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well known to those skilled in the art.

The pharmaceutical compositions to be used for in vivo administration should be sterile. This is readily accomplished by, for example, filtration through sterile filtration membranes. Sterile injectable solutions are generally prepared by incorporating the active (e.g., AAV vectors, AAV particles) in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The pharmaceutical compositions disclosed herein may also comprise other ingredients such as diluents and adjuvants. Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycols.

Any of the compositions (e.g., AAV vectors, AAV particles, AAV genomes) described herein can be used for alleviating and/or treating a disease or a condition. Thus, in some aspects, the present disclosure provides methods for alleviating one or more symptoms and/or for treating a disease or a condition in a subject in need of the treatment by compositions disclosed herein, as well as a pharmaceutical compositions comprising such. In some aspects, a subject of the methods herein may be a human subject. In some aspects, the subject may be a juvenile human subject, such a human subject under the age of 18 years. In some aspects, the subject may be a human subject under the age of 5, 4, 3, 2 or 1 year. In some aspects, the subject may be a subject that has not been previously exposed to wild-type AAV or a recombinant (rAAV) vector. In some aspects, the subject may be a subject that has not been previously administered a rAAV vector. In some aspects, the subject is a subject that has been previously administered a rAAV vector, e.g., arAAV vector described herein. A subject that has been exposed or administered an AAV or rAAV can be identified using methods known in the art, e.g., by PCR detection of viral DNA or by measuring antibody titer to AAV or rAAV, either the capsid or the transgene. In some aspects, the subject may be a subject that has not been administered an enzyme replacement therapy (e.g., by administration of the enzyme protein). A subject that has been administered an enzyme replacement therapy can be identified using methods known in the art, e.g., by measuring antibody titer to the enzyme. However, in some aspects the subject has previously been treated with an enzyme replacement therapy. In some aspects, the subject is a subject that has undergone one or more approaches to clear neutralizing antibodies (NAbs) (e.g., plasmapheresis, immunosuppression, enzymatic degradation). In some aspects, a subject suitable of methods of use herein may not need to clear neutralizing antibodies (NAbs) before administration of any of the compositions (e.g., AAV vectors, AAV particles, AAV genomes) described herein.

In some aspects, the subject has or is suspected of having a disease that may be treated with gene therapy. Illustrative diseases or a conditions that can be treated using the methods disclosed herein can include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., a, β, γ], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping (see, e.g., WO/2003/095647), antisense against U7 snRNAs to induce exon skipping (see, e.g., WO/2006/021724), and antibodies or antibody fragments against myostatin or myostatin propeptide) and Becker, Gaucher disease (glucocerebrosidase), Hurler's disease (a-L-iduronidase), adenosine deaminase deficiency (adenosine deaminase), glycogen storage diseases (e.g., Fabry disease [a-galactosidase] and Pompe disease [lysosomal acid a-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., IIC), serca2a, zinc finger proteins that regulate the phospholamban gene, Barkct, P2-adrenergic receptor, p2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like.

To perform the methods disclosed herein, a therapeutically effective amount of the compositions (e.g., AAV vectors, AAV particles, AAV genomes) or a pharmaceutical composition comprising such may be administered to a subject who needs treatment via a suitable route (e.g., intramuscular, intravenous, intracerebroventricular injection, intra-cisterna magna injection, intravitreal, subretinal, subconjuctival, retrobulbar, intracameral, suprachoroidal, intracoronary injection, intraarterial injection, and/or intra-parenchymal injection) at a suitable amount as disclosed herein.

In certain aspects, the present disclosure also provides for methods of introducing a nucleic acid molecule into a cell, comprising contacting the cell with a virus vector and/or composition disclosed herein. In some aspects, methods herein can include delivering a nucleic acid molecule herein to a cell, comprising contacting the cell or layer with a viral vector disclosed herein wherein the viral vector comprises the nucleic acid molecule of interest. In some aspects of this method, a nucleic acid molecule of interest can encode a therapeutic protein or therapeutic RNA. In some aspects, the therapeutic protein can be a monoclonal antibody or a fusion protein.

In certain aspects, the present disclosure also provides for methods of introducing a nucleic acid molecule to a CNS tissue, a heart tissue, a liver tissue, a skeletal muscle tissue, or any combination thereof, comprising contacting the cell with a virus vector and/or composition disclosed herein. In some aspects, nucleic acid molecules herein can be delivered to a specific tissue by administering AAV particles having one or more nucleic acid molecules herein to a CNS tissue, a heart tissue, a liver tissue, a skeletal muscle tissue, or any combination thereof.

In some aspects, methods of administering at least one AAV particle or at least one AAV vector having one or more nucleic acid molecules herein to a tissue substantially modulates expression of the at least one protein and/or gene as compared to baseline. As used herein, “baseline” refers to the expression of the at least one transgene (and the encoded product of the transgene) before the AAV particle or AAV vector having one or more nucleic acid molecules was administered. As used herein, “substantially modulates expression” refers to at least a 1-fold change in expression (e.g., increased expression, decreased expression) as compared to baseline. In some aspects, methods of administering at least one AAV particle or AAV vector having one or more nucleic acid molecules herein to a tissue modulates expression of the at least one protein and/or gene as compared to baseline by at least about 2-fold to about 50-fold (e.g., about 2-, 4-, 6-, 8-, 10-, 20-, 30-, 40-, 50-fold). In some aspects, methods of administering at least one AAV particle or AAV vector having one or more nucleic acid molecules herein to a tissue modulates expression of the at least one protein and/or gene as compared to baseline by at least about 2-fold to about 50-fold (e.g., about 2-, 4-, 6-, 8-, 10-, 20-, 30-, 40-, 50-fold) when the at least one AAV particle or AAV vector is delivered to a CNS tissue, a heart tissue, a liver tissue, a skeletal muscle tissue, or any combination thereof.

In any of the methods disclosed herein, an effective amount of the compositions (e.g., AAV vectors, AAV particles, AAV genomes) described herein can be given to a subject in need thereof to alleviate one or more symptoms associated with a disease and or condition. “An effective amount” as used herein refers to a dose of a disclosed composition which is sufficient to confer a therapeutic effect on a subject having a disease and or condition. In some aspects, an effective amount can be an amount that reduces at least one symptom of disease or condition in the subject.

In some aspects, methods of administering at least one AAV particle or at least one AAV vector as disclosed herein can have increased gene transfer efficiency in a cell compared to naturally isolated AAV vectors. In some aspects, methods of administering at least one AAV particle or at least one AAV vector as disclosed herein can have at least about 2-fold to about 50-fold (e.g., about 2-, 4-, 6-, 8-, 10-, 20-, 30-, 40-, 50-fold) increased gene transfer efficiency in a cell compared to naturally isolated AAV vectors. In some aspects, methods of administering at least one AAV particle or at least one AAV vector as disclosed herein can have increased gene transfer efficiency in a tissue compared to naturally isolated AAV vectors. In some aspects, methods of administering at least one AAV particle or at least one AAV vector as disclosed herein can have at least about 2-fold to about 50-fold (e.g., about 2-, 4-, 6-, 8-, 10-, 20-, 30-, 40-, 50-fold) increased gene transfer efficiency in a tissue compared to naturally isolated AAV vectors. In some aspects, methods of administering at least one AAV particle or at least one AAV vector as disclosed herein can have increased gene transfer efficiency in a subject compared to naturally isolated AAV vectors. In some aspects, methods of administering at least one AAV particle or at least one AAV vector as disclosed herein can have at least about 2-fold to about 50-fold (e.g., about 2-, 4-, 6-, 8-, 10-, 20-, 30-, 40-, 50-fold) increased gene transfer efficiency in a subject compared to naturally isolated AAV vectors.

In some aspects, methods herein may include administering at least one AAV particle and/or at least one AAV vector to a subject at least once. In some aspects, methods herein may include administering at least one AAV particle and/or at least one AAV vector to a subject more than once. In some aspects, methods herein may include administering at least one AAV particle and/or at least one AAV vector to a subject between at least once to at least 10 times (e.g., about 1, 2, 3, 4, 5, 6, 7, 8, 9, 10 times). In some aspects, methods herein may include administering at least one AAV particle and/or at least one AAV vector to a subject at least twice, at least 3 times, at least 4 times, or at least 5 times. In some aspects, methods herein may include administering at least one AAV particle and/or at least one AAV vector to a subject once a day, once every other day, once a week, once every two weeks, once every three weeks, once a month, once every other month, once every three months, once every four months, once a year, or twice a year.

The present disclosure also provides kits for use in preparing any one of the compositions (e.g., or AAV vectors, AAV particles, AAV genomes) as described herein and kits having one or more therapeutic uses as described herein. A kit for use as described herein may include one or more containers further including a composition (e.g., or AAV vectors, AAV particles, AAV genomes) as described herein, formulated in a pharmaceutical composition.

In some aspects, the kit can additionally comprise instructions for use of compositions (e.g., or AAV vectors, AAV particles, AAV genomes) in any of the methods described herein. The included instructions may include a description of administration of the compositions or a pharmaceutical composition comprising such to a subject to achieve the intended activity in a subject. The kit may further comprise a description of selecting a subject suitable for treatment based on identifying whether the subject is in need of the treatment. The instructions relating to the use of the compositions as described herein generally include information as to dosage, dosing schedule, and route of administration for the intended treatment.

The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port.

Kits optionally may provide additional components such as buffers and interpretive information. Normally, the kit comprises a container and a label or package insert(s) on or associated with the container. In some aspects, the disclosure provides articles of manufacture comprising contents of the kits described above.

### EXAMPLES

While the present disclosure has been described with reference to the specific aspects thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Materials and Methods

The materials and methods disclosed herein were used in one or more the Examples.

Birdman-8 vector packaging CBA-Fluc was produced using HEK293 E1A cells and a triple transfection method (pHelper, pRepCap, and pTransfer). Here, Birdman-8 RepCap was synthesized by GenScript. Snakeman-8 and Dragonman-8 were synthesized with gBlocks (Integrated DNA Technologies). Each RepCap sequence was then cloned into the pLH8 plasmid using the HindIII site in rep and the XbaI site in pLH8 polyA. Birdman-2 was generated using PCR overlap extension using pLH-Birdman-8 and pLH2. DA1-VP1u-AAV8, DA1-PLA2-AAV8, and DAI-Nterm-AAV8 were generated using HiFi DNA assembly (New England Biolabs) with pLH-Avian-DA1 and pLH8.

AAV was produced by transfecting HEK293 E1A cells, grown to 80% confluence in 15 cm cell culture dishes. Each dish was transfected with 12 μg adenovirus helper plasmid (pXX680), 10 μg RepCap plasmid, and 6 μg ITR transfer plasmid. Transfer plasmids used include pTR-CBA-Fluc, pTR-sc-CBh-eGFP, and pTR-CBh-Luc-vsfGFP-9. Transfection was accomplished using PEI (Poly Sciences) at a ratio of 3.5 μg PEI per μg DNA. Cells were incubated in a 37° C. CO₂ incubator. Media was collected on days 3/4 and 6/7 post-transfection and frozen at −80° C.

Media was centrifuged at 2,400 g for 5 minutes to remove cell debris. PEG-8000 was added to the media to a final concentration of 8%. PEG-media incubated at 4° C. overnight. Next, PEG-media was centrifuged at 4,000 g for 1 hour to pellet PEG along with the viral vector. Vector was resuspended in 2 mL DNase buffer (10 mM tris-HCL, 10 mM MgCl₂, 2 mM CaCl₂)) and treated with 20 μL DNase I (10 mg/mL). Next, an iodixanol gradient was prepared by layering 3 mL 17% iodixanol, 3 mL 25% iodixanol, 4 mL 40% iodixanol, and 3 mL 60% iodixanol in a clear ultracentrifuge tube (Beckman-Coulter). Vector solution was overlaid on top of the gradient and centrifuged at 100,000 g for 15 hours. The top two layers were discarded and 1 mL Iodixanol fractions were then collected. Fractions were titered with quantitative PCR titered to determine AAV genome concentration. Peak fractions were then pooled and purified by desalting (Thermo Fisher Zeba Spin Desalting Column [40k]) or diafiltration (Thermo Fisher Pierce Protein Concentrator PES [100k]). Final vector preps were eluted in PBS containing 0.001% pluronic F68. Final vector concentrations were determined by quantitative PCR.

AAV samples were treated with DNase I, along with standards and negative controls, to remove non-capsid packaged DNA. 10 μL of each sample was combined with 89 μL DNase buffer (10 mM tris-HCL, 10 mM MgCl₂, 2 mM CaCl₂)) and 1 μL DNase I (10 mg/mL) and incubated at 37° C. for 1 hour. Samples were then treated with 6 μL EDTA (0.5 M) to inactivate DNase I. Next, samples were diluted 1:2 with 10% Tween-20, to inhibit non-specific genome-capsid interactions. Samples were then diluted 1:100 to achieve a final Tween-20 concentration of 0.05%. Next, standards were subject to 7 8-fold dilutions in H₂O. Finally, 2 μL of each sample was combined with 5 μL SYBR Green qPCR Master Mix (Thermo Fisher), 2.5 μL H₂O, and 0.5 μL primer mix (10 μM each forward and reverse primer). Concentrations were then determined by quantitative PCR with primers amplifying AAV2 inverted terminal repeat regions.

### Example 1

### Generation of AAV Chimera Capsid Proteins

Capsid proteins of AAV variants have multiple domains in the VP1 unique (VP1u) and VP1/2 shared regions of the capsid that play important roles in trafficking and, ultimately, transduction. VP1u contains a phospholipase A2 domain used in endosomal escape, and there are multiple basic-rich (BR) regions that act as nuclear localization signals (NLSs) spread across the capsid. BR1 is located in VP1u after the PLA2 domain, BR2 and BR3 are in the VP1/2 shared region, and BR4 is in VP3. The area of VP1u before the PLA2 domain has been shown to mediate some kind of interaction with the host protein GPR108 in most primate AAVs, which is essential for transduction in these serotypes.

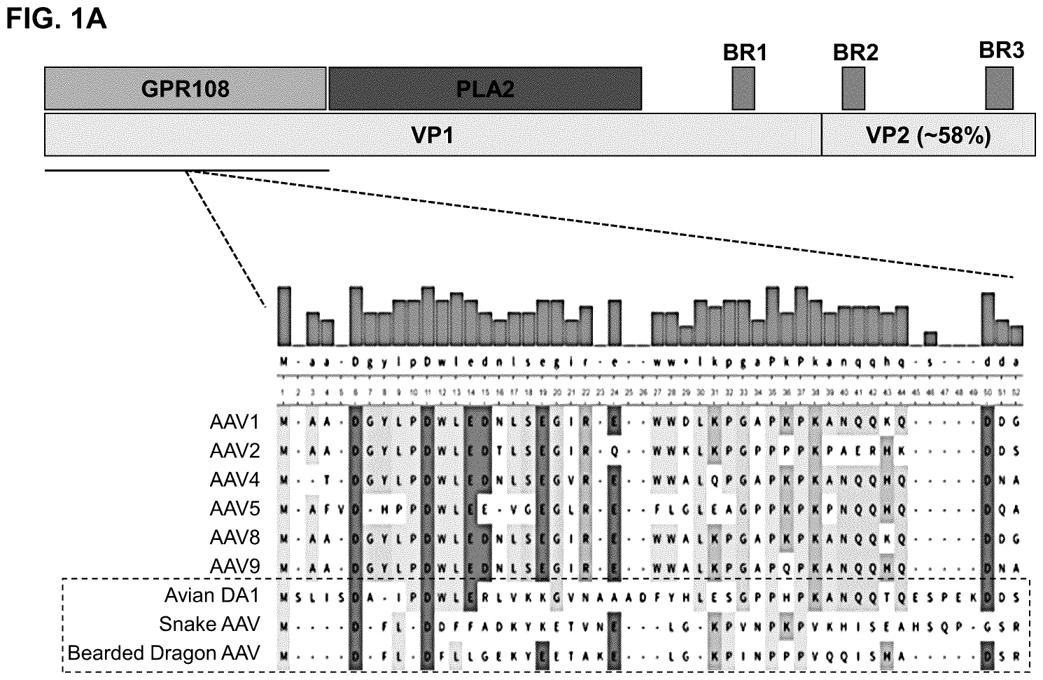

Sequences for avian DA-1 (accession YP_077183), snake (accession YP_068094.1), and bearded dragon (accession YP_009154713) AAVs were obtained from NCBI and aligned with primate AAV sequences using the UGENE program according to previously described methods (Okonechnikov et al. (2012) Bioinformatics. 28:1166-1167), the disclosure of which is incorporated herein in its entirety. Sequence alignment of reptile AAVs with those known to infect human cells revealed two areas of significantly lower homology between the two groups (FIG. 1A and FIG. 1B). The VP1 region contained a region shown to be important for interaction with essential host factor GPR108, the phospholipase A2 domain (PLA2), and one of four “basic rich” (BR) domains, which function as nuclear localization signals. The partial VP2 sequence (38 amino acids, or about 58% of VP2) contains the BR2 and BR3 domains. BR3 has been shown to be especially important for transduction. The alignment shown in FIG. 1A and FIG. 1B included AAV serotypes 1, 2, 3, 4, 5, 8, 9, avian DA-1, snake, and bearded dragon, in that order. Highlighting in FIG. 1A and FIG. 1B indicates sequence conservation among the serotypes. The reptilian serotypes (avian DA-1, snake, and bearded dragon), shown FIG. 1A and FIG. 1B inside the dashed box, did not differ significantly from the other serotypes in the PLA2 domain; however, the sequences did differ significantly in the GPR108-interacting region (FIG. 1A) and the BR2 and BR3 domains in VP2 (FIG. 1B). Specifically, the region of VP1u before the PLA2 domain, which is responsible for interactions with the host protein GPR108, and the area of VP2 containing the BR2 and BR3 NLS domains bore little resemblance to similar areas of reptile AAVs. Interestingly, AAV5 and AAV4 similarly showed fairly high divergence in VP1 and VP2, respectively, and these serotypes are known to infect human cells less efficiently.

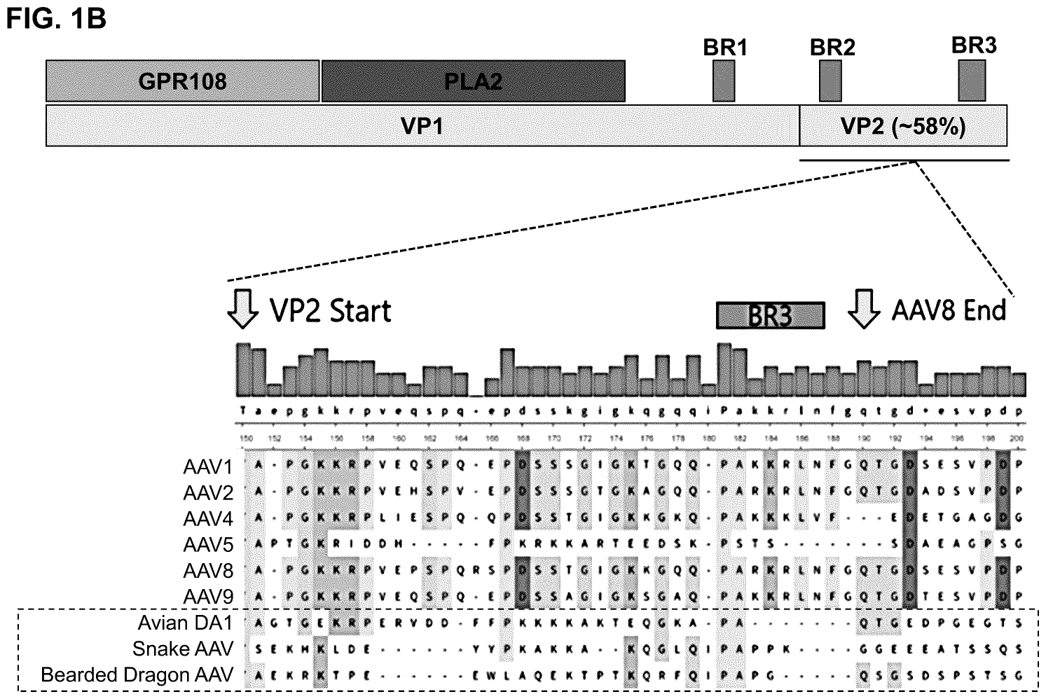

Next, chimera capsid proteins were constructed. The BM8 chimera capsid protein construct was a fusion between AAV8 and avian AAV strain DA-1. Specifically, the BM8 capsid was constructed by grafting the VP1/2 region of AAV8 onto avian AAV strain DA-1, up until the beginning of the AAP reading frame to not disrupt production of this essential protein (FIG. 2A). Snake AAV and bearded dragon AAV were used to make SM8 and DM8 chimera capsid protein constructs, respectively, by also fusing with AAV8 at the beginning of the AAP reading frame (FIG. 2B). In brief, BM8, SM8, and DM8 reptile (Avian DA-1, Bearded Dragon, and Snake) and chimeric (BM8, DM8, SM8) cap DNA sequences were synthesized and cloned in a pLH8 plasmid, which contained the AAV2 rep gene and the AAV8 cap gene, using the HindIII site in rep and an XbaI site that followed the cap polyA signal. Some examples herein described mutated BM8 chimera capsid protein constructs-BM2 and BM8L-which were created by PCR generating homologous overhangs using the appropriate templates (pLH2 and pLH-DA1 for BM2, pLH8 and pLH-DA1 for BM8L), followed by overlap extension.

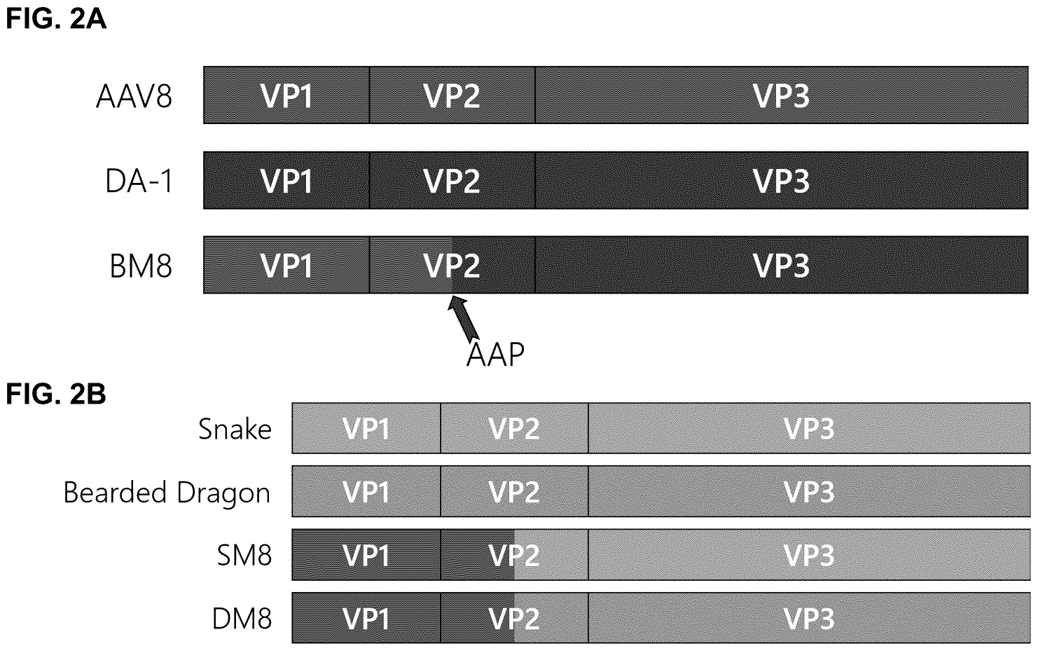

The BM8 chimera capsid was not recognized by the anti-AAV2 monoclonal antibody B1, which binds at the C-terminus of VP3, while AAV8 has the full B1 epitope and was recognized (FIG. 3A). BM8 was recognized, however, by the anti-AAV2 monoclonal antibody A1, which recognized a linear epitope in VP1. Avian AAV was only weakly recognized by the antibody, as it contained one amino acid difference in this region (FIG. 3B). AAV8 and BM8 both contained the full epitope, so they were fully recognized (FIG. 3B). Interestingly, the DA-1 and BM8 VP1 proteins ran at a lower apparent molecular weight than those of AAV8, potentially indicating a difference in post-translational modification.

### Example 2

### AAV Vector Production and Characterization

Recombinant AAV vectors were produced by transfecting HEK293 cells at 70-80% confluence with polyethylenimine using the triple-plasmid transfection protocol. In brief, on the day of transfection, the cells were counted using a ViCell XR Viability Analyzer (Beckman Coulter) and diluted to 1×10⁶ viable cells/mL. To mix the transfection cocktail, the following reagents were added in this order: plasmid DNA (CsCl purified), Optimem (Thermo Fisher Scientific), Optipro or F17 media, Polyethyleneimine Max (MW 25,000). The plasmid DNA used also included AAV helper plasmids and 1.5 μg total plasmid was used per 1×10⁶ total cells. The pXR series of helper plasmids were used to generate multiple rAAV serotype vectors. PEI Max was used at a PEI:DNA ratio of 2:1. Optimem I, Optipro, or F17 media was added such that the entire transfection mix cocktail made up 5% of the total flask final volume. The cocktail was vortexed for 5-10 seconds prior to being incubated at room temperature for 10-15 minutes. The transfection cocktail was then pipetted into the flasks and placed back in the shaker/incubator. Three hours post-transfection, the flasks were supplemented with CDM4HEK293 media (HyClone, Logan, UT) at 10% of final total cell culture volume to halt the transfection and prevent further PEI-related cell aggregation. Cells were harvested 48 hours post-transfection with viabilities ranging between 75 and 90% depending on input plasmids.

Recombinant vectors packaging single-stranded genomes encoding firefly luciferase driven by the chicken β-actin promoter (ssCBA-Luc) or self-complementary green fluorescence protein driven by a hybrid chicken β-actin promoter (scCBh-GFP) were generated using the methods described above. Vector purification was carried out using an iodaxinol gradient ultracentrifugation protocol, buffer exchange and concentration using vivaspin2 100 kDa molecular weight cut-off (MWCO) centrifugation columns (F-2731-100 Bioexpress, Kaysville, UT). Recombinant AAV vector titers were determined by quantitative PCR with primers amplifying AAV2 inverted terminal repeat regions, 5′-AACATGCTACGCAGAGAGGGAGTGG-3′ (SEQ ID NO:96) and 5′-CATGAGACAAGGAACCCCTAGTGATGGAG-3′ (SEQ ID NO:97).

### 1. Characterization of BM8 Vector

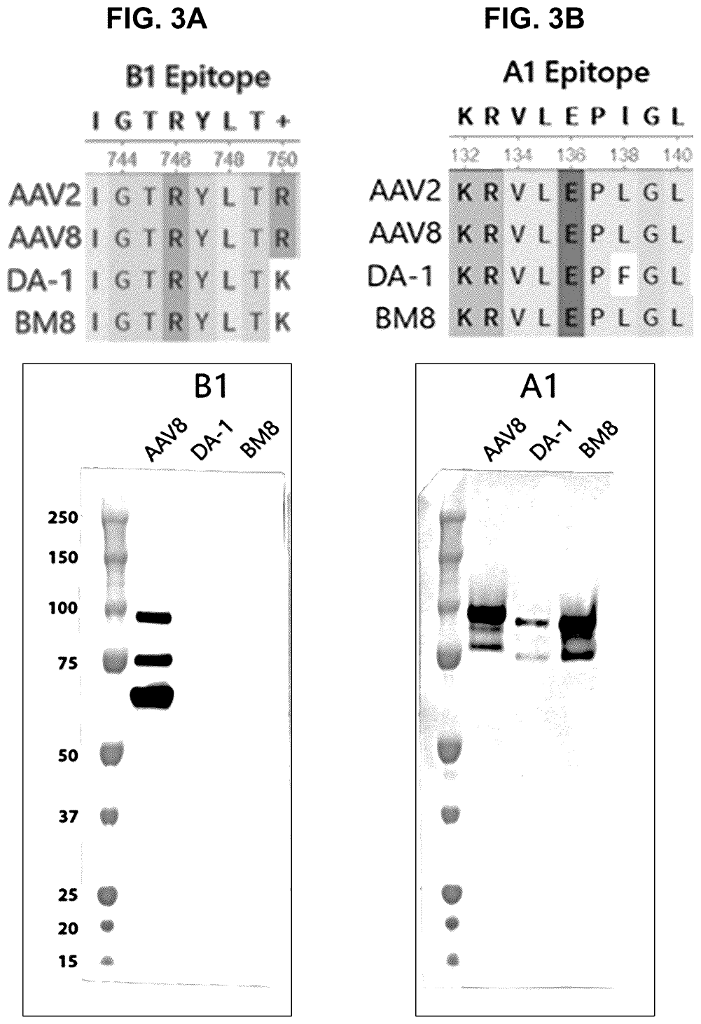

AAV luciferase vectors were produced as described above and used to transduce HEK293 cells at medium (10,000) and high (100,000) MOIs. For transduction assays, viral vectors packaging ssCBA-Luc were diluted in 1×PBS containing 200 mM NaCl, 1 mM MgCl₂, and 0.001% Pluronic F68. Cells were incubated with vectors at the indicated MOI for 48 hours at 37° C. in 5% CO₂. Cells were lysed by incubation in 1× Passive Lysis Buffer (Promega) for 30 minutes at room temperature. Luciferase activity was measured with a Victor X3 plate reader (PerkinElmer) immediately following the addition of 25 μL of luciferin (Promega). While DA-1 showed no transduction at the medium MOI, and only miniscule transduction at the higher MOI, BM8 transduction was comparable to AAV8 or higher (FIG. 4A).

Evasion of pre-existing anti-AAV neutralizing antibodies was evaluated using serum from a dog treated with AAV8 vector. For serum neutralization assays, viral vectors packaging ssCBA-Luc and anti-AAV8 serum were diluted in serum-free DMEM and equal volumes (25 μL each) were combined in black, clear-bottom 96-well plates (Corning) and incubated at room temperature for 30 minutes. HEK293 cells were added to each well (10,000 cells in 50 μL DMEM 10% FBS) and incubated at 37° C. in 5% CO₂ for 24 hours (100,000 vg/cell). While the engineered AAV hum.8 variant (engineered to evade anti-AAV8 antibodies) showed improved escape compared to AAV8, it was still strongly neutralized at low serum dilutions (FIG. 4B). Strikingly, BM8 maintained robust transduction even at a 1:5 dilution of serum, suggesting it could possibly be used in patients previously treated with AAV vectors.

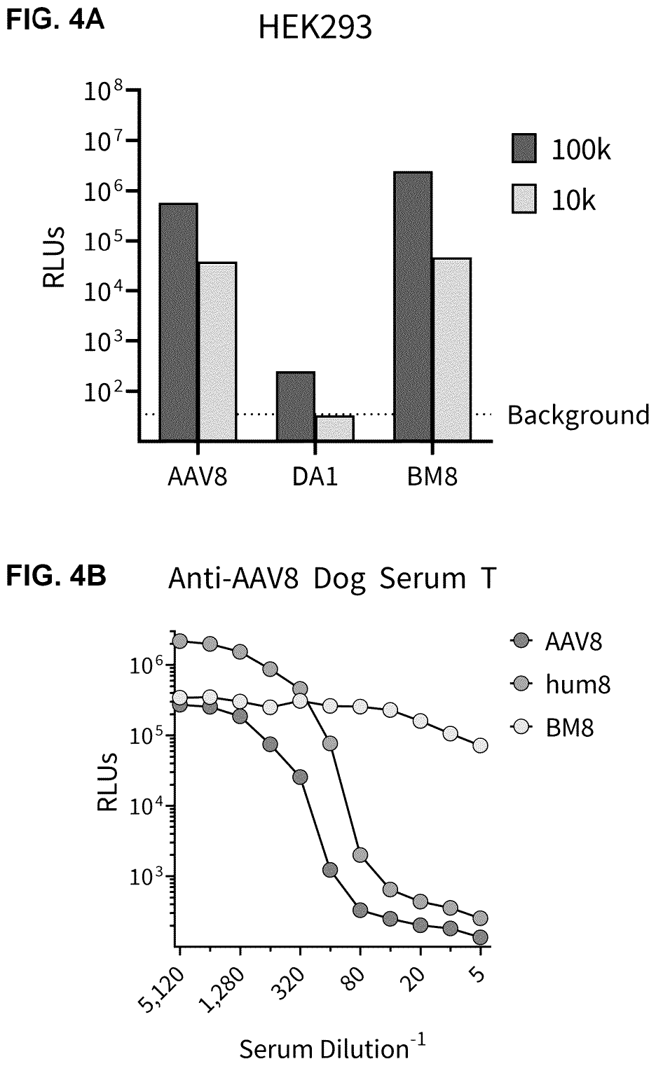

To determine the importance of different portions of the AAV8 graft, as well as explore the use of other primate serotypes, two additional mutants were constructed as described in Example 1. The BM8L mutant contained the VP1u region of AAV8, but none of the VP1/2 shared region (FIG. 5A). The BM2 variant mirrors BM8, but contains a sequence grafted from AAV2 rather than AAV8 (FIG. 5A). Transduction of U87 cells with these constructs showed an approximately 80% decrease in transduction for BM8L compared to BM8, while transduction of BM2 was not substantially different (FIG. 5B). While changing the VP1/2 region from AAV8 to AAV2 did not affect transduction (BM8 vs. BM2), reverting the VP2 portion back to Avian AAV dropped transduction over 80% (BM8 vs. BM8L). This suggested that the 38 amino acids from VP2 are crucial for the full BM8 phenotype.

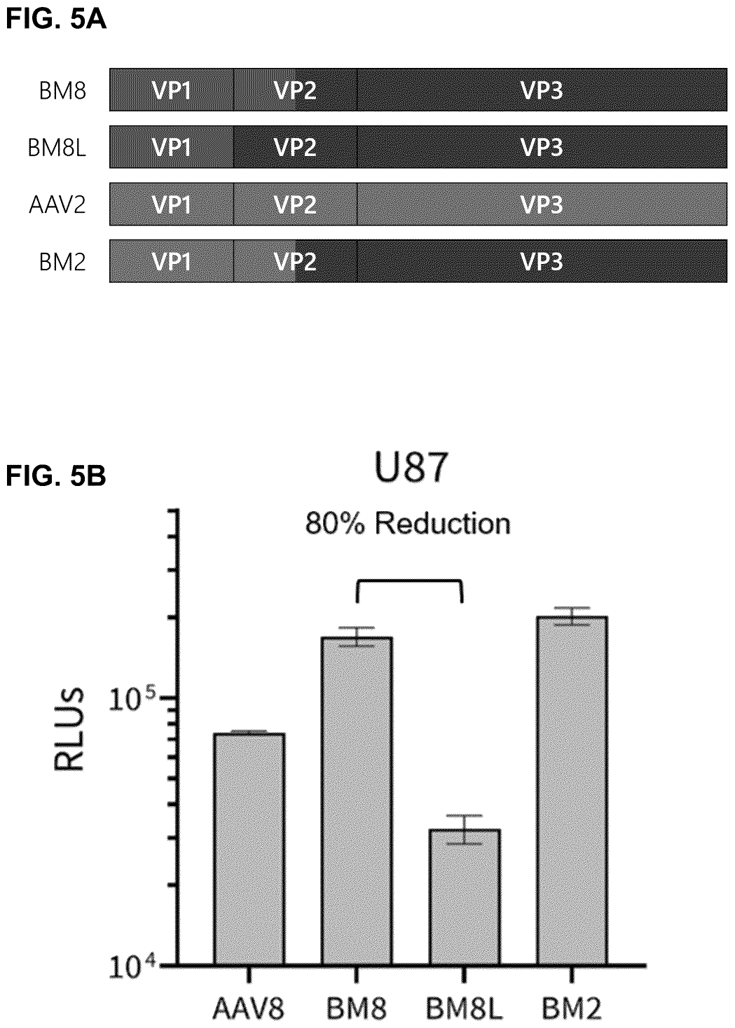

The BM8 construct was also characterized in vivo. In brief, naïve C57BL/6 mice were injected at around 12 weeks of age with AAV9-GFP vector (3e11 vg/mouse) or PBS. Four weeks later, AAV-luciferase vectors were administered. Four weeks after the second injection, mice were sacrificed, and organs were harvested. Around 30 mg of each tissue was lysed in 200 μL 1× Passive Lysis Buffer (Promega) with metal beads using a Qiagen TissueLyser II. Lysates were used to measure luciferase activity as described above. BM8 showed transduction in the mouse heart while Avian DA-1 did not (FIG. 6).

### 2. Characterization of SM8 and DM8 Vectors

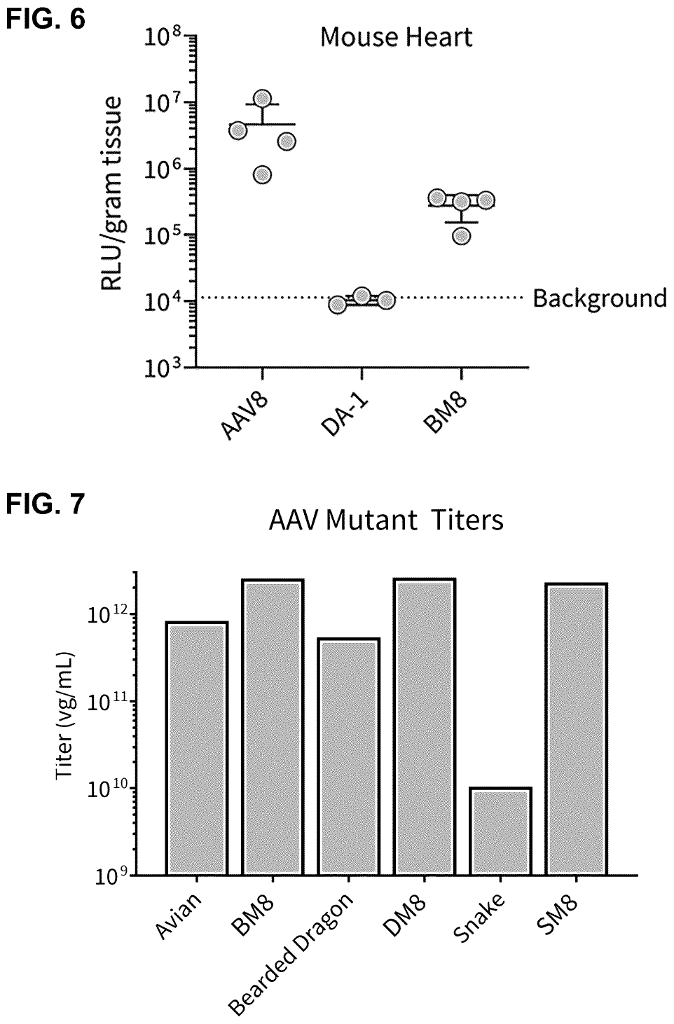

As described above, vectors were constructed by grafting AAV8 VP1/2 onto Snake and Bearded Dragon AAVs (FIG. 2B). AAV-luciferase vectors were produced according to methods described herein. Final titer after purification is shown in FIG. 7. Chimeric capsids showed a consistent increase in titer compared to the natural, non-mammalian versions. Snake AAV produced near-background titers and was not evaluated further.

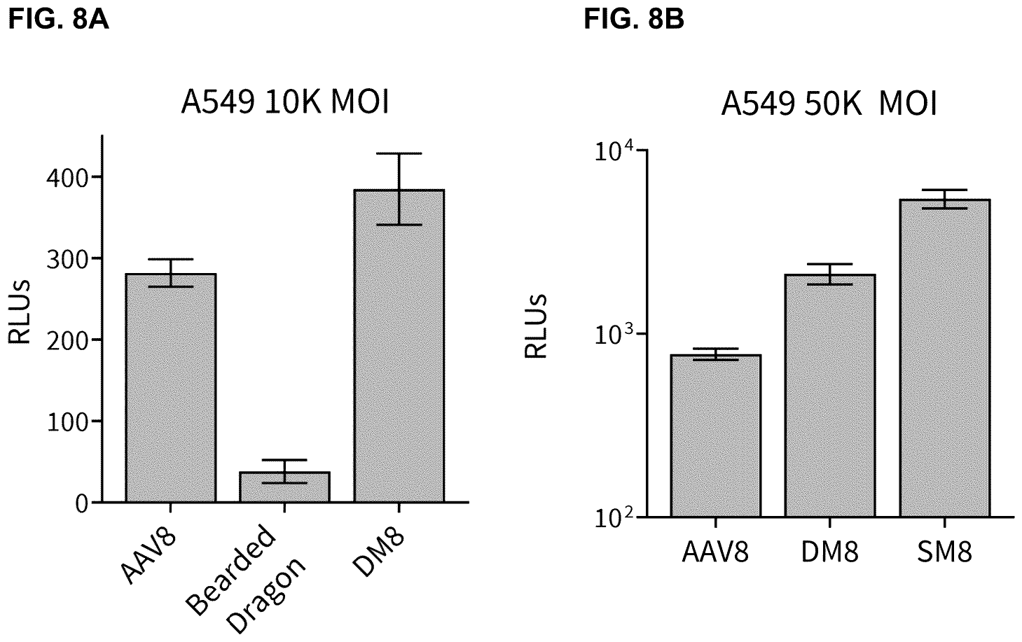

The vectors displayed differential transduction of cell lines compared to BM8, so A549 cells were transduced with the AAV-luciferase vectors using the methods disclosed herein. As shown in FIG. 8A, Bearded Dragon AAV did not transduce above background at 10k MOI, while DM8 transduced better than AAV8. The difference between them was more apparent at a higher MOI, and SM8 transduced even better than DM8 (FIG. 8B).

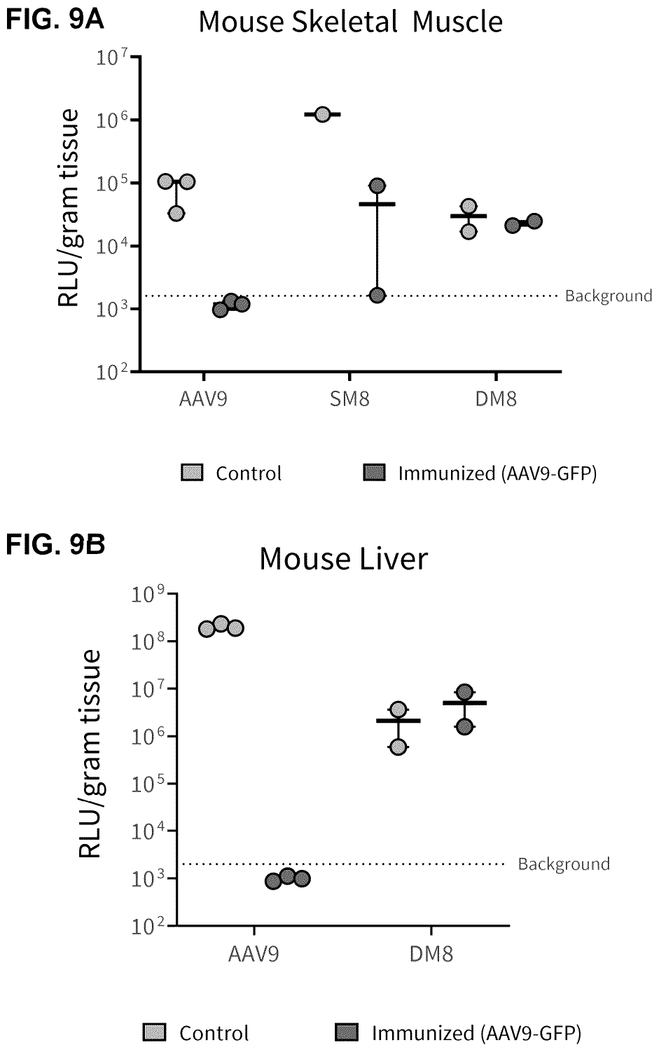

The SM8 and DM8 vectors were also evaluated in mice, both for transduction and evasion of pre-existing neutralizing antibodies. Mice were pre-immunized with AAV9-GFP or PBS, followed by administration of AAV-luciferase vectors four weeks later using the methods described above. Animals were sacrificed four weeks following AAV-luciferase administration and harvested organs were assayed for luciferase expression. Strikingly, while AAV9-luciferase was completely neutralized following AAV9-GFP immunization, DM8 and SM8 maintained efficient transduction despite the presence of anti-AAV9 neutralizing antibodies in immunized animals (FIG. 9A and FIG. 9B). Further, the tropism of both DM8 and SM8 appears to be specific in mice. Transduction was negligible in heart and lung. DM8 transduction was split between liver and skeletal muscle. In liver, DM8 transduction was well below AAV9, while DM8 performance was within the lower range of AAV9 transduction in skeletal muscle. SM8 was even more specific; transduction was limited to skeletal muscle, where SM8 performed as well as AAV9 or better. Both capsids far outperformed AAV9 following pre-immunization, suggesting that DM8 and/or SM8 may be useful for administering a second dose of gene therapy to patients previously treated with AAV vectors, in addition to enabling treatment of patients with neutralizing Abs generated as a result of natural AAV exposure.

### Example 3

### DA-1 Successfully Assembled Capsids and Packaged Genomes

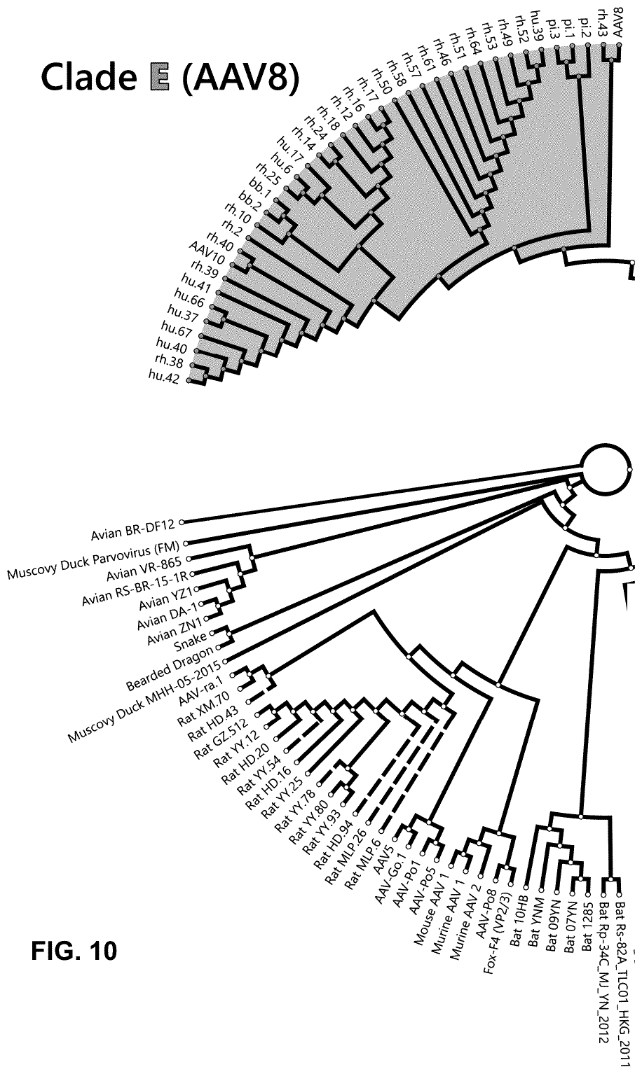

With only 60% pairwise amino acid identity between DA-1 and AAV8 VP3 (FIG. 10), there should be significantly different capsid surface topologies and, thus, low antibody cross-reactivity. To test this hypothesis, a DA-1 vector packaging CBA-Fluc using HEK293 E1A cells was generated and a triple transfection method (pHelper, pRepCap, and pTransfer) was used. As a positive control, an AAV8 vector packaging CBA-Fluc was generated.

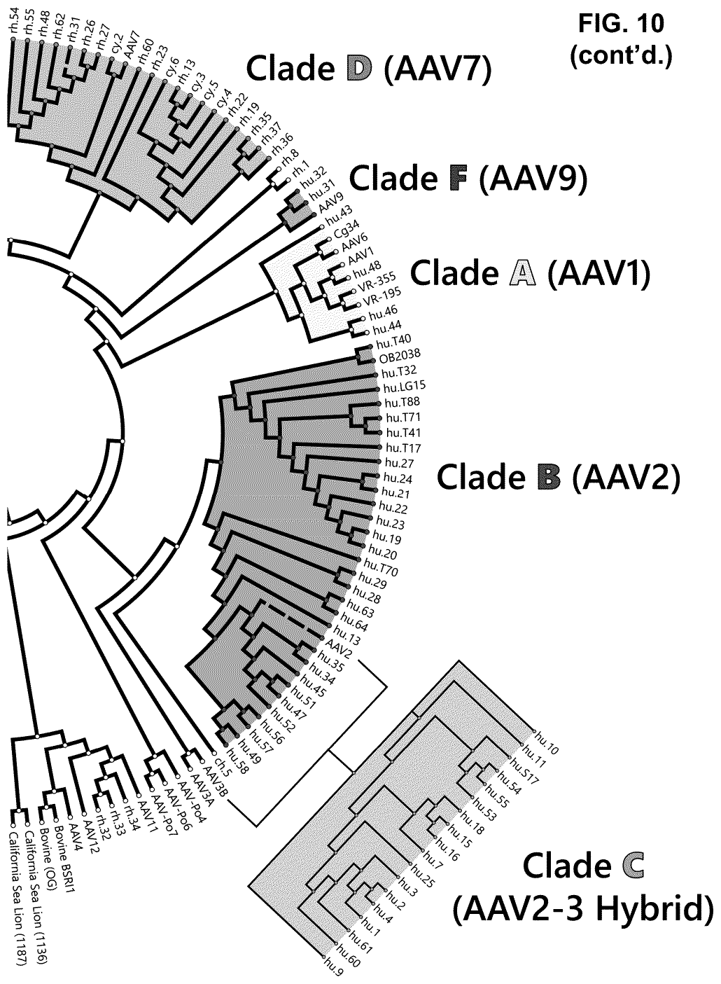

HEK293 E1A cells were seeded in 96-well plates at a density of 1e5 cells/mL and grown in a 37° C. CO₂ incubator overnight. Each well was transfected with 0.12 μg adenovirus helper plasmid (pXX680), 0.10 μg RepCap plasmid, and 0.06 μg ITR transfer plasmid (pTR-sc-CBh-eGFP). Transfection of AAV vector was accomplished using PEI (PolySciences) at a ratio of 3.5 μg PEI per μg DNA. Cells were incubated in a 37° C. CO₂ incubator. 24 hours after transfection, media was discarded and replaced to remove untransfected DNA. At 6 days post-transfection, the media and cells were collected from three wells for each vector and frozen at −80° C. Cells were then treated with passive lysis buffer (Promega), DNase I, RNase I, and proteinase inhibitor for 30 minutes. Packaged vector yields for each sample was determined by quantitative PCR using primers against the ITRs. As shown in in FIG. 11, cell and media yields were summed to determine total packaged vector yield. The packaging assay demonstrated that DA-1 successfully assembled capsids and packaged genomes.

### Example 4

### BM8 Successfully Assembled Capsids and Packaged Genomes

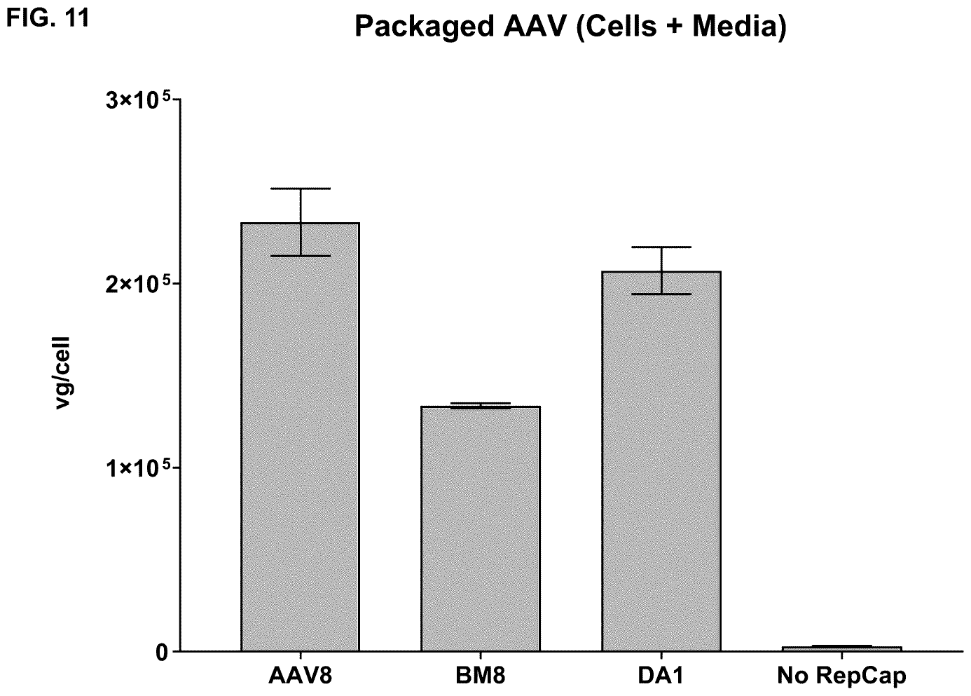

However, a luciferase transduction assay shows that DA-1 transduction is fully inhibited in a range of mammalian cell lines. HeLa, A549, U87, HEK293, HuH7, C2C12, and TH1 cells were seeded in 96-well plates at a density of 1e5 cells/mL and grown in a 37° C. CO₂ incubator overnight. Three wells per cell line were used to establish a cell count. AAV vector packaging CBh-Luc-vsfGFP-9 was diluted in PBS (0.001% pluronic F68) to the appropriate concentration for an MOI of 1e5 vg/cell. AAV vector was then added to each well and allowed to incubate at 37° C. for 48 hours. Media was aspirated from each well and cells were then treated with 20 μL 1× passive lysis buffer (Promega) for 30 minutes. Next, 20 μL firefly luciferin was added to each well. As shown in FIG. 12, Luminescence was then measured for a duration of 1 second by the VICTOR X Plate Reader (PerkinElmer).

### Example 5

### DA-1 Bound to the Cell Surface and was Internalized

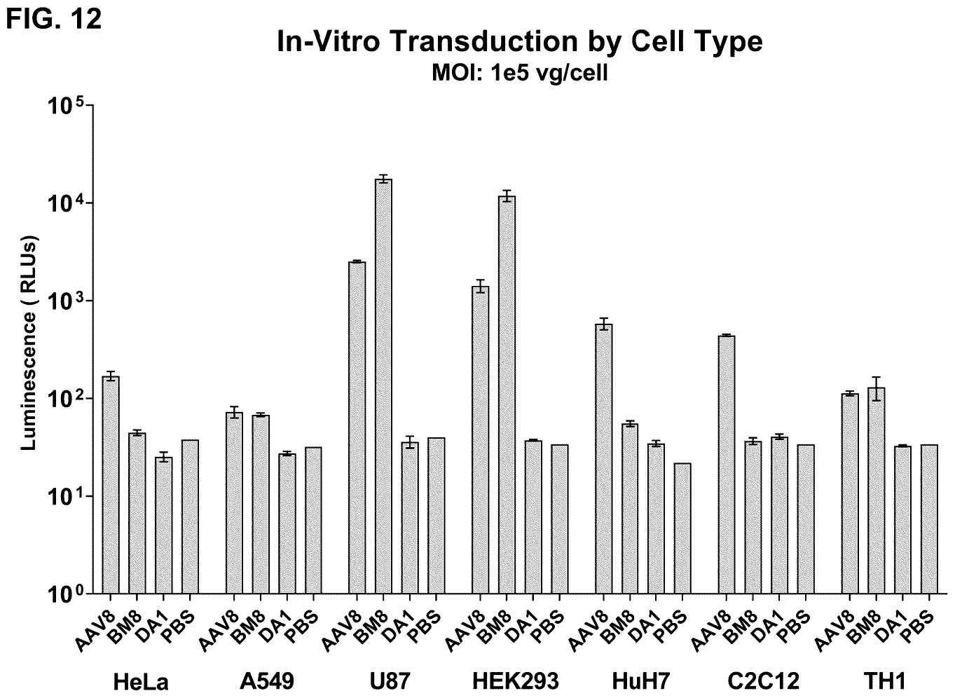

Despite failing to transduce mammalian cells, a binding and uptake assay reveals that DA-1 binds the cell surface and is internalized as levels similar to AAV8. For the cell binding assay (FIG. 13A), U87 cells were seeded into four 6-well plates at a density of 3e5 cells/mL and grown in a 37° C. CO₂ incubator overnight. One well per plate was used to establish a cell count. Cells were then preincubated at 4° C. for 30 minutes. AAV vector packaging CBA-Fluc was diluted in PBS (0.001% pluronic F68) to the appropriate concentration for an MOI of 2e4 vg/cell. AAV vector was then added to each well and allowed to incubate at 4° C. for 1 hour. Media was aspirated and cells were collected and washed three times with PBS to remove unbound vector. Cells were vortexed vigorously with lysis buffer (10 mM tris-HCl, 10 mM MgCl₂, 2 mM CaCl₂, 0.5% Triton X-100) and then treated with RNase I, and proteinase inhibitor for 1 hour. AAV titers were then determined by quantitative PCR for each sample using primers against the luciferase transgene.

For the cell uptake assay (FIG. 13B), U87 cells were seeded into 4 6-well plates at a density of 3e5 cells/mL and grown in a 37° C. CO₂ incubator overnight. One well per plate was used to establish a cell count. AAV vector packaging CBA-Fluc was diluted in PBS (0.001% pluronic F68) to the appropriate concentration for an MOI of 2e4 vg/cell. AAV vector was then added to each well and allowed to incubate at 37° C. for 1 hour. Cells were collected and washed three times with PBS to remove excess vector. Cells were vortexed vigorously with lysis buffer (10 mM tris-HCl, 10 mM MgCl₂, 2 mM CaCl₂, 0.5% Triton X-100) and then treated with RNase I, and proteinase inhibitor for 1 hour. AAV titers were then determined by quantitative PCR for each sample using primers against the luciferase transgene. FIG. 13C shows the percent of bound vector internalized was calculated by dividing cell binding vg/cell by cell uptake vg/cell.

### Example 6

### Generation and Characterization of Birdman-8

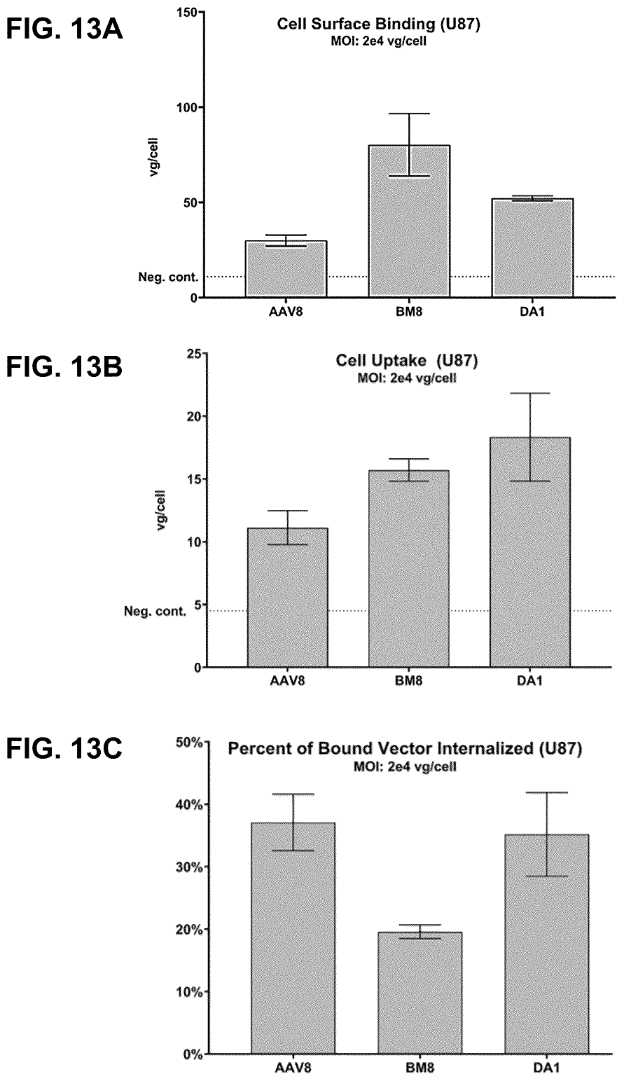

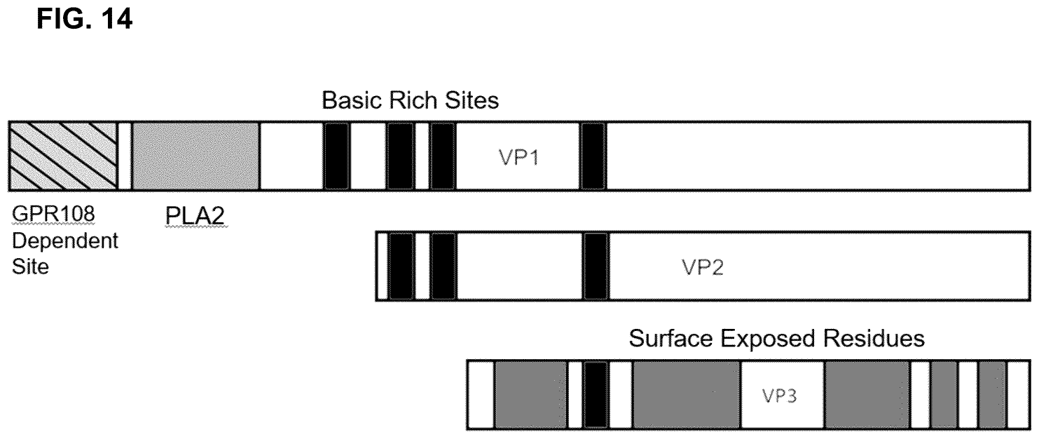

To rescue transduction in DA-1 without altering immune evasion, a DA1-AAV8 hybrid capsid was generated by fusing DA-1 and AAV8 at the VP1/2 unique region. Given the binding and uptake data, it appeared that the intracellular function of DA-1 was impaired. The VP1/2 unique region of AAV has previously been linked with the intracellular function of AAV (FIG. 14). Moreover, the first surface-exposed residue of AAV does not begin until VP3 (FIG. 14). Thus, to rescue transduction in DA-1 without impacting its antigenic profile, the VP1/2 unique region of DA-1 was swapped for that of AAV8. To generate this new hybrid vector, termed Birdman-8, the first 177 amino acids of Avian AAV DA-1 VP1 were replaced with the first 175 amino acids of AAV8 [YP_077180.1] VP1 (FIG. 15).

A packaging assay again validated that Birdman-8 can successfully assemble capsids and packages genomes (FIG. 11). Further, a luciferase transduction assay showed that our VP1/2 swap rescued transduction in DA-1 (FIG. 12). Birdman-8 (BM8) transduced significantly above background levels in several cell lines: A549, U87, HEK293, HuH7, and TH1. Further, Birdman-8 significantly outperformed AAV8 transduction in U87 and HEK293. Birdman-8 transduced U87 cells 7.03-fold (p=0.0116) better and HEK293 cells 8.37-fold (p=0.0203) better than AAV8.

After demonstrating the transduction profile of Birdman-8 (BM8), its ability to avoid immune evasion in vitro and in vivo was evaluated. To test the immune evasion of Birdman-8 against AAV8 in vitro, the vectors were pre-incubated with varying concentrations of anti-AAV8 dog serum T. Transduction of the vectors was then again measured in HEK293 cells using a luciferase reporter gene. Viral vectors packaging CBA-Fluc and anti-AAV8 serum were diluted in serum-free DMEM and equal volumes (25 μL each) were combined in black, clear-bottom 96-well plates (Corning) and incubated at room temperature for 30 minutes. HEK293 cells were added to each well (10,000 cells in 50 μL DMEM 10% FBS) and incubated at 37° C. in 5% CO₂ for 24 hours (100,000 vg/cell). Cells were lysed by incubation in 1× Passive Lysis Buffer (Promega) for 30 minutes at room temperature. Luciferase activity was measured with a Victor X3 plate reader (PerkinElmer) immediately following the addition of 25 μL of luciferin (Promega) to 25 μL of cell lysate.

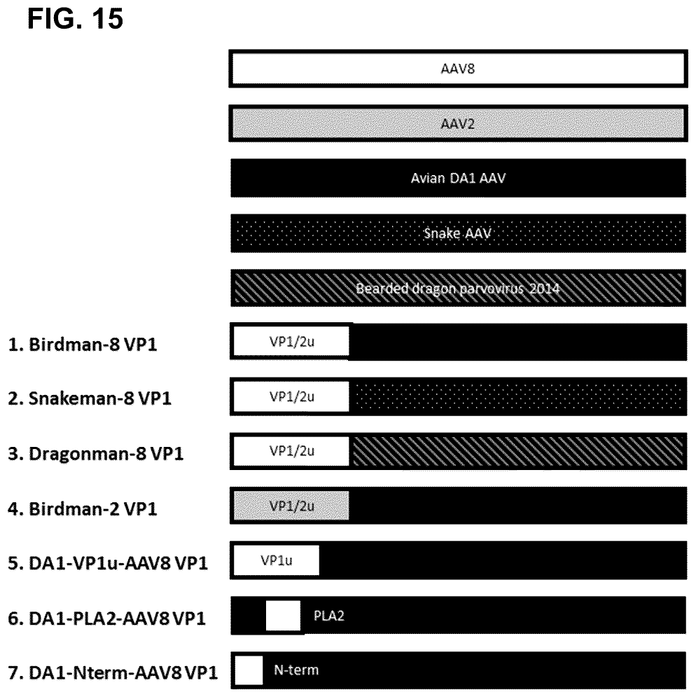

FIG. 16 shows that while AAV8 transduction was only 0.80-fold lower than Birdman-8 at a 5120⁻¹ serum dilution, transduction was 0.001-fold lower at an 80⁻¹ serum dilution. Further, Birdman-8 transduction dropped only 0.76-fold between a 5120⁻¹ serum dilution and 80⁻¹ dilution. In contrast, AAV8 transduction fell 0.001-fold. This indicates that Birdman-8 robustly evaded anti-AAV8 antibodies in vitro. Birdman-8 and AAV8 therefore exhibited little antibody cross-reactivity.

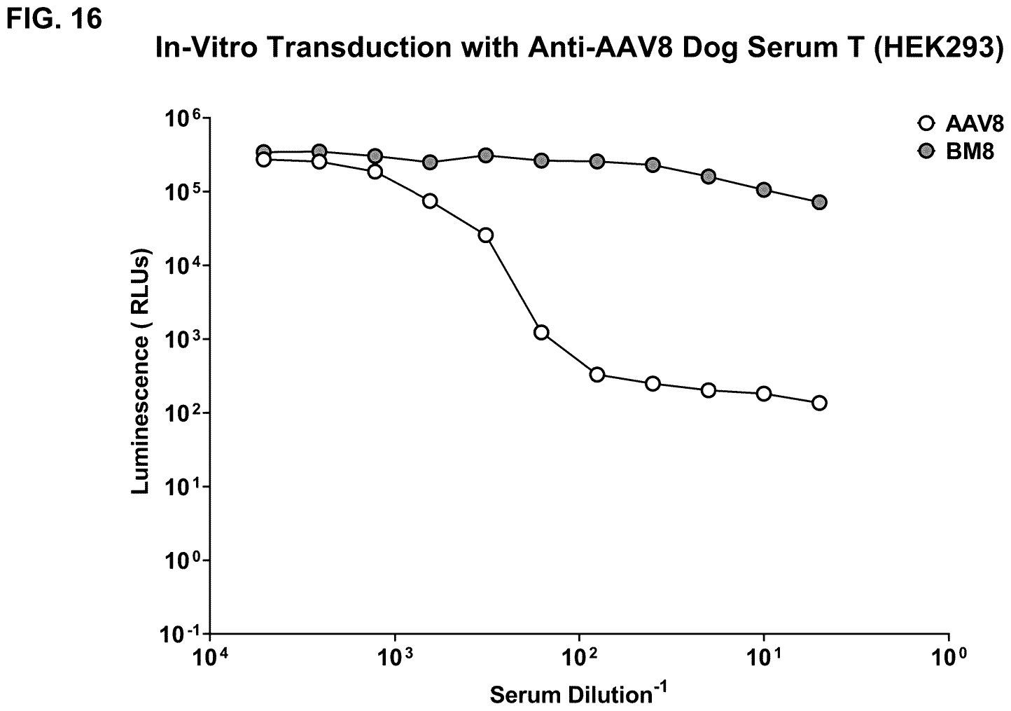

Next, the immune evasion of Birdman-8 in vivo was examined. Naïve C57BL/6 mice were injected at around 12 weeks of age with AAV9-GFP vector (3e11 vg/mouse) or PBS. Four weeks later, AAV-luciferase vectors (AAV9 or Birdman-8) were administered. Four weeks after the second injection, mice were sacrificed, and organs were harvested. Around 30 mg of each tissue was lysed in 200 μL Passive Lysis Buffer (Promega) with metal beads using a Qiagen TissueLyser II. Luciferase activity was measured with a Victor X3 plate reader (PerkinElmer) immediately following the addition of 25 μL of luciferin (Promega) to 25 μL of tissue lysate. As shown in FIG. 17A-FIG. 17C, transduction was measured in liver, heart, and skeletal muscle tissue via luciferase assay. In the unimmunized mice, AAV9 transduction markedly outperformed Birdman-8. In the AAV9-GFP immunized mice, however, Birdman-8 transduction outperformed AAV9 in all tested tissue types. Birdman-8 transduction was 1.71-fold greater in liver tissue, 5.64-fold greater in cardiac tissue, and 8.08-fold greater in skeletal muscle. This indicated that Birdman-8 robustly evaded anti-AAV9 antibodies in vivo. Birdman-8 and AAV9, therefore exhibit little antibody cross-reactivity.

### Example 7

### Generation and Characterization of Snakeman-8

With the promising results from our immune evasion experiments, whether VP1/2 swaps could be used to rescue transduction in other non-mammalian dependoparvoviruses was examined. If the answer was YES, then it would be possible to generate a profile of antigenically distinct, immune-evasive capsids. Thus, two more hybrid vectors, Snakeman-8 and Dragonman-8, were generated. To clone Snakeman-8, the first 177 amino acids of Snake Adeno-associated virus VP1 [YP_068094.1] were replaced with the first 175 amino acids of AAV8 VP1 (FIG. 15). Likewise, Dragonman-8 was cloned by replacing the first 177 amino acids of Bearded dragon parvovirus 2014 [YP_009154713.1] with the first 175 amino acids of AAV8 VP1 (FIG. 15). Snakeman-8 and Dragonman-8 vectors packaging CBA-Fluc were then produced using HEK293 E1A cells and a triple transfection method.

The sequences for the constructs shown in FIG. 15 are presented below.

Amino Acid Sequence (Sequence Name // Sequence Identifier)

MAADGYLPDWLEDNLSEGIREWWALKPGAPKPKANQQKQDDGRGLVLPGYKYLGP
FNGLDKGEPVNAADAAALEHDKAYDQQLQAGDNPYLRYNHADAEFQERLQEDTSF
GGNLGRAVFQAKKRVLEPLGLVEEGAKTAPGKKRPVEPSPQRSPDSSTGIGKKGQQP
ARKRLNFGQTGEDPGEGTSSNAGSSAPSSVGSSVMAEGGGGPMGDAGQGADGVGNS
SGNWHCDSQWLDNGVVTRTTRTWVLPSYNNHLYKRIQGPGGTDPNNKFFGFSTPW
GYFDYNRFHCHFSPRDWQRLINNNWGIRPKAMRFRLFNIQVKEVTVQDSNTTIANNL
TSTVQVFADKDYQLPYVLGSATEGTFPPFPADIYTIPQYGYCTLNYNNEAVDRSAFYC
LDYFPSDMLRTGNNFEFTYTFEDVPFHSMFAHNQTLDRLMNPLVDQYLWAFSSVSQT
GSSGRALNYSRATKTNMATQYRNWLPGPFVRDQQIFTGASNITQNNVFNVWDKGKQ
WVIDNRINMMQPGPAAATTFSGEPDRQAMQNTLAFSRTVYDQTTSTTDRNQLLITNE
DEIRPTNSVGIDTWGVVPNNNQSKVTAGTRAAINNQGALPGMVWQNRDIYLQGPIW
AKIPDTDNHFHPSPLIGGFGCKHPPPQIFIKNTPVPANPSETFQTAKVASFINQYSTGQC
TVEIFWELKKETSKRWNPEIQFTSNFGNAADIQFAVSDTGSYSEPRPIGTRYLTKPL
(Birdman-8 // SEQ ID NO: 01)
MAADGYLPDWLEDNLSEGIREWWALKPGAPKPKANQQKQDDGRGLVLPGYKYLGP
FNGLDKGEPVNAADAAALEHDKAYDQQLQAGDNPYLRYNHADAEFQERLQEDTSF
GGNLGRAVFQAKKRVLEPLGLVEEGAKTAPGKKRPVEPSPQRSPDSSTGIGKKGQQP
ARKRLNFGQTGEEEATSSQSGGSPAGSDTSGTSVMATGGGGPMADDNQGAEGVGNS
SGDWHCDTKWMGDHVITKSTRTWVLPTYGNHLYGPINFDGTTGSGANAAYAGYKT
PWGYFDFNRFHCHFSPRDWQRLINNHTGIRPKGLKIKVFNVQVKEVTTQDSTKTIAN
NLTSTVQIFADENYDLPYVLGSATQGTFPPFPNDVFMLPQYAYCTLQGNSGKFVDRS
AFYCLEYFPSQMLRTGNNFEFQFKFEEVPFHSGWAQSQSLDRLMNPLLDQYLIGDYG
TDASGNLIYHRAGPNDLNEFYKNWAPAPYECIQNINSSDNTKNANSINGSNSTNKWG
LQGRQAWDAPGFVQASTYEGAAAGQSLLNGVLTFDKSSATTSSPAATAVNRTIEDEI
QGTNNFGNARNNIVAINQQTKGTNPTTGSTSQFETMPGMVWSNRDIYLQGPIWAKIP
NTDGHFHPSPRMGGFGLKHPPPMILIKNTPVPADPPTTFNPMPQTSFITEYSTGQVTVE
MLWEVQKESSKRWNPEVQFTSNFGTSDPAVDGIPFGINNLGTYVESRPIGTRYISKHL
(Snakeman-8 // SEQ ID NO: 02)
MAADGYLPDWLEDNLSEGIREWWALKPGAPKPKANQQKQDDGRGLVLPGYKYLGP
FNGLDKGEPVNAADAAALEHDKAYDQQLQAGDNPYLRYNHADAEFQERLQEDTSF
GGNLGRAVFQAKKRVLEPLGLVEEGAKTAPGKKRPVEPSPQRSPDSSTGIGKKGQQP
ARKRLNFGQTGSDSPSTSGSGGTAGSSSSASNTMAQGGGGPMADDNQGAEGVGNAS
GDWHCDTQWLGDHVITKSTRTWVLPSYGNHLYSPINFDGTTGNGTQAAYCGYATP
WAYFDFNRFHCHFSPRDWQRLINNHTGIRPVGLKFKLFNIQVKEITVQDSTKTIANNL
TSTVQVFADTEHQLPYVLGNATQGTFPPFPAEVFQLPQYGYCTIQGPQGKFTDRSAFY
CLEYFPSKMLRTGNNFEFTYKFEKVPFHSGWAQSQSLDRLMNPLIPQYLLADYGTTA
SSAITYYRPNSTDLSWYFKNWLPGPVERRQQINSEDSTKNHANLNGNAHTNKYSIQH
RQTKMMPGVALSSKYTGAAEGTSLLNGVLTFDKIANDNTNITDTNNVNRTIEDEIQG
TNPYGNDVPITVAVNTQNATTSPTMQNSSTYELLPGAVWSNRDIYLQGPIWAKIPDT
DGHFHPSPQMGGFGLKNPPPMILIKNTPVPADPPTTFKPTPMNSFISEYSTGQVTVEML
WEVRKEDSKRWNPEIQFTSNFGLSDPQIDGIPFGITKLGNYEEPRPIGSRYLSKHL
(Dragonman-8 // SEQ ID NO: 03)
MAADGYLPDWLEDTLSEGIRQWWKLKPGPPPPKPAERHKDDSRGLVLPGYKYLGPF
NGLDKGEPVNEADAAALEHDKAYDRQLDSGDNPYLKYNHADAEFQERLKEDTSFG
GNLGRAVFQAKKRVLEPLGLVEEPVKTAPGKKRPVEHSPVEPDSSSGTGKAGQQPAR
KRLNFGQTGEDPGEGTSSNAGSSAPSSVGSSVMAEGGGGPMGDAGQGADGVGNSSG
NWHCDSQWLDNGVVTRTTRTWVLPSYNNHLYKRIQGPGGTDPNNKFFGFSTPWGY
FDYNRFHCHFSPRDWQRLINNNWGIRPKAMRFRLFNIQVKEVTVQDSNTTIANNLTS
TVQVFADKDYQLPYVLGSATEGTFPPFPADIYTIPQYGYCTLNYNNEAVDRSAFYCL
DYFPSDMLRTGNNFEFTYTFEDVPFHSMFAHNQTLDRLMNPLVDQYLWAFSSVSQT
GSSGRALNYSRATKTNMATQYRNWLPGPFVRDQQIFTGASNITQNNVFNVWDKGKQ
WVIDNRINMMQPGPAAATTFSGEPDRQAMQNTLAFSRTVYDQTTSTTDRNQLLITNE
DEIRPTNSVGIDTWGVVPNNNQSKVTAGTRAAINNQGALPGMVWQNRDIYLQGPIW
AKIPDTDNHFHPSPLIGGFGCKHPPPQIFIKNTPVPANPSETFQTAKVASFINQYSTGQC
TVEIFWELKKETSKRWNPEIQFTSNFGNAADIQFAVSDTGSYSEPRPIGTRYLTKPL
(Birdman-2 // SEQ ID NO: 04)
MAADGYLPDWLEDNLSEGIREWWALKPGAPKPKANQQKQDDGRGLVLPGYKYLGP
FNGLDKGEPVNAADAAALEHDKAYDQQLQAGDNPYLRYNHADAEFQERLQEDTSF
GGNLGRAVFQAKKRVLEPLGLVEEGAKTAGTGEKRPERVDDFFPKKKKAKTEQGKA
PAQTGEDPGEGTSSNAGSSAPSSVGSSVMAEGGGGPMGDAGQGADGVGNSSGNWH
CDSQWLDNGVVTRTTRTWVLPSYNNHLYKRIQGPGGTDPNNKFFGFSTPWGYFDYN
RFHCHFSPRDWQRLINNNWGIRPKAMRFRLFNIQVKEVTVQDSNTTIANNLTSTVQV
FADKDYQLPYVLGSATEGTFPPFPADIYTIPQYGYCTLNYNNEAVDRSAFYCLDYFPS
DMLRTGNNFEFTYTFEDVPFHSMFAHNQTLDRLMNPLVDQYLWAFSSVSQTGSSGR
ALNYSRATKTNMATQYRNWLPGPFVRDQQIFTGASNITQNNVFNVWDKGKQWVID
NRINMMQPGPAAATTFSGEPDRQAMQNTLAFSRTVYDQTTSTTDRNQLLITNEDEIRP
TNSVGIDTWGVVPNNNQSKVTAGTRAAINNQGALPGMVWQNRDIYLQGPIWAKIPD
TDNHFHPSPLIGGFGCKHPPPQIFIKNTPVPANPSETFQTAKVASFINQYSTGQCTVEIF
WELKKETSKRWNPEIQFTSNFGNAADIQFAVSDTGSYSEPRPIGTRYLTKPL (DA1-
VP1u-AAV8 // SEQ ID NO: 05)
MSLISDAIPDWLERLVKKGVNAAADFYHLESGPPHPKANQQTQESPEKDDSRGLVLP
GYKYLGPFNGLDKGEPVNAADAAALEHDKAYDQQLQAGDNPYLRYNHADAEFQER
LKDDTSFGGNLGKAIFQAKKRVLEPFGLIEQPDNTAGTGEKRPERVDDFFPKKKKAK
TEQGKAPAQTGEDPGEGTSSNAGSSAPSSVGSSVMAEGGGGPMGDAGQGADGVGNS
SGNWHCDSQWLDNGVVTRTTRTWVLPSYNNHLYKRIQGPGGTDPNNKFFGFSTPW
GYFDYNRFHCHFSPRDWQRLINNNWGIRPKAMRFRLFNIQVKEVTVQDSNTTIANNL
TSTVQVFADKDYQLPYVLGSATEGTFPPFPADIYTIPQYGYCTLNYNNEAVDRSAFYC
LDYFPSDMLRTGNNFEFTYTFEDVPFHSMFAHNQTLDRLMNPLVDQYLWAFSSVSQT
GSSGRALNYSRATKTNMATQYRNWLPGPFVRDQQIFTGASNITQNNVFNVWDKGKQ
WVIDNRINMMQPGPAAATTFSGEPDRQAMQNTLAFSRTVYDQTTSTTDRNQLLITNE
DEIRPTNSVGIDTWGVVPNNNQSKVTAGTRAAINNQGALPGMVWQNRDIYLQGPIW
AKIPDTDNHFHPSPLIGGFGCKHPPPQIFIKNTPVPANPSETFQTAKVASFINQYSTGQC
TVEIFWELKKETSKRWNPEIQFTSNFGNAADIQFAVSDTGSYSEPRPIGTRYLTKPL
(DA1-VP1u-AAV8 // SEQ ID NO: 06)
MAADGYLPDWLEDNLSEGIREWWALKPGAPKPKANQQKQDDGRGLVFPGYKYLGP
FNGLDKGKPVNEADAAALEHDKAYDLELKDGHNPYFEYNEADRRFQERLKDDTSFG
GNLGKAIFQAKKRVLEPFGLIEQPDNTAGTGEKRPERVDDFFPKKKKAKTEQGKAPA
QTGEDPGEGTSSNAGSSAPSSVGSSVMAEGGGGPMGDAGQGADGVGNSSGNWHCD
SQWLDNGVVTRTTRTWVLPSYNNHLYKRIQGPGGTDPNNKFFGFSTPWGYFDYNRF
HCHFSPRDWQRLINNNWGIRPKAMRFRLFNIQVKEVTVQDSNTTIANNLTSTVQVFA
DKDYQLPYVLGSATEGTFPPFPADIYTIPQYGYCTLNYNNEAVDRSAFYCLDYFPSDM
LRTGNNFEFTYTFEDVPFHSMFAHNQTLDRLMNPLVDQYLWAFSSVSQTGSSGRALN
YSRATKTNMATQYRNWLPGPFVRDQQIFTGASNITQNNVFNVWDKGKQWVIDNRIN
MMQPGPAAATTFSGEPDRQAMQNTLAFSRTVYDQTTSTTDRNQLLITNEDEIRPTNS
VGIDTWGVVPNNNQSKVTAGTRAAINNQGALPGMVWQNRDIYLQGPIWAKIPDTDN
HFHPSPLIGGFGCKHPPPQIFIKNTPVPANPSETFQTAKVASFINQYSTGQCTVEIFWEL
KKETSKRWNPEIQFTSNFGNAADIQFAVSDTGSYSEPRPIGTRYLTKPL (DA1-Nterm-
AAV8 // SEQ ID NO: 07)

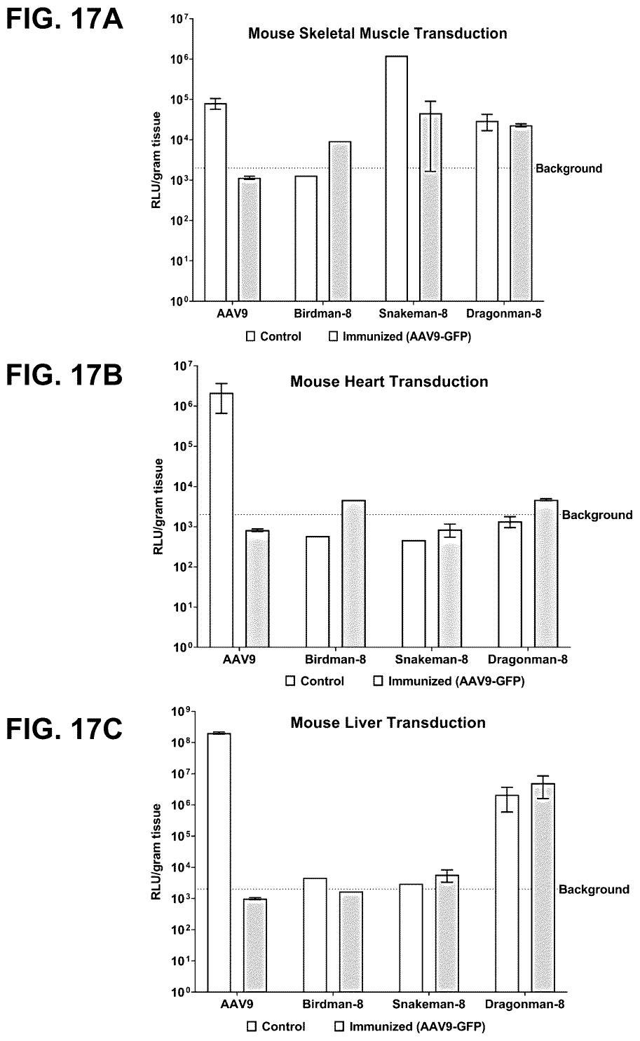

The vectors packaging CBA-Fluc were diluted in 1×PBS containing 200 mM NaCl, 1 mM MgCl₂, and 0.001% Pluronic F68. A549 cells were incubated with vectors at the indicated MOI for 48 hours at 37° C. in 5% CO₂. Cells were lysed by incubation in 1× Passive Lysis Buffer (Promega) for 30 minutes at room temperature. Luciferase activity was measured with a Victor X3 plate reader (PerkinElmer) immediately following the addition of 25 μL of luciferin (Promega) to 25 μL of cell lysate. The luciferase transduction assay demonstrated that Snakeman-8 and Dragonman-8 were capable of transducing human cells and outperforming AAV8 (FIG. 18). Snakeman-8 transduced A549 cells 7.06-fold (p=0.0016) better than AAV8. Dragonman-8 transduced A549 cells 2.75-fold (p=0.0006) better than AAV8. Thus, the ability of VP1/2 swaps to rescue transduction was not limited to AAV8. Rather, a wide range of sauropsidan (avian, reptile, etc.) dependoparvoviruses may be amenable to rescue by this approach.

Next, the immune evasion of Snakeman-8 and Dragonman-8 was investigated as was the transduction in vivo. Naïve C57BL/6 mice were injected at around 12 weeks of age with AAV9-GFP vector (3e11 vg/mouse) or PBS. Four weeks later, AAV-luciferase vectors were administered. Four weeks after the second injection, mice were sacrificed, and organs were harvested. Around 30 mg of each tissue was lysed in 200 μL Passive Lysis Buffer (Promega) with metal beads using a Qiagen TissueLyser II. Luciferase activity was measured with a Victor X3 plate reader (PerkinElmer) immediately following the addition of 25 μL of luciferin (Promega) to 25 μL of tissue lysate. Transduction was measured in liver (FIG. 17A), heart (FIG. 17B), and skeletal muscle tissue (FIG. 17A) via luciferase assay. In immunized liver tissue, Dragonman-8 transduced 5058-fold greater than AAV9 and Snakeman-8 transduced 5.83-fold greater than AAV9. In immunized heart tissue, Dragonman-8 transduced 5.73-fold greater than AAV9 while Snakeman-8 transduced at similar levels. Finally, in immunized skeletal muscle tissue, Dragonman-8 transduced 19.89-fold greater than AAV9 and Snakeman-8 transduced 39.78-fold greater than AAV9.

### Example 8

### Generation and Characterization of Birdman-2

Whether DA-1 transduction could be rescued using VP1/2 swaps from other mammalian AAVs besides AAV8 was examined. Thus, a new vector, Birdman-2 (SEQ ID NO:04), was generated by replacing the first 177 amino acids of DA-1 VP1 with the first 174 amino acids of AAV2 [YP_680426.1] VP1 (FIG. 15). A Birdman-2 vector packaging CBA-Fluc was then produced using HEK293 E1A cells and a triple transfection method was used.

Then, vectors packaging CBA-Fluc were diluted in 1×PBS containing 200 mM NaCl, 1 mM MgCl₂, and 0.001% Pluronic F68.

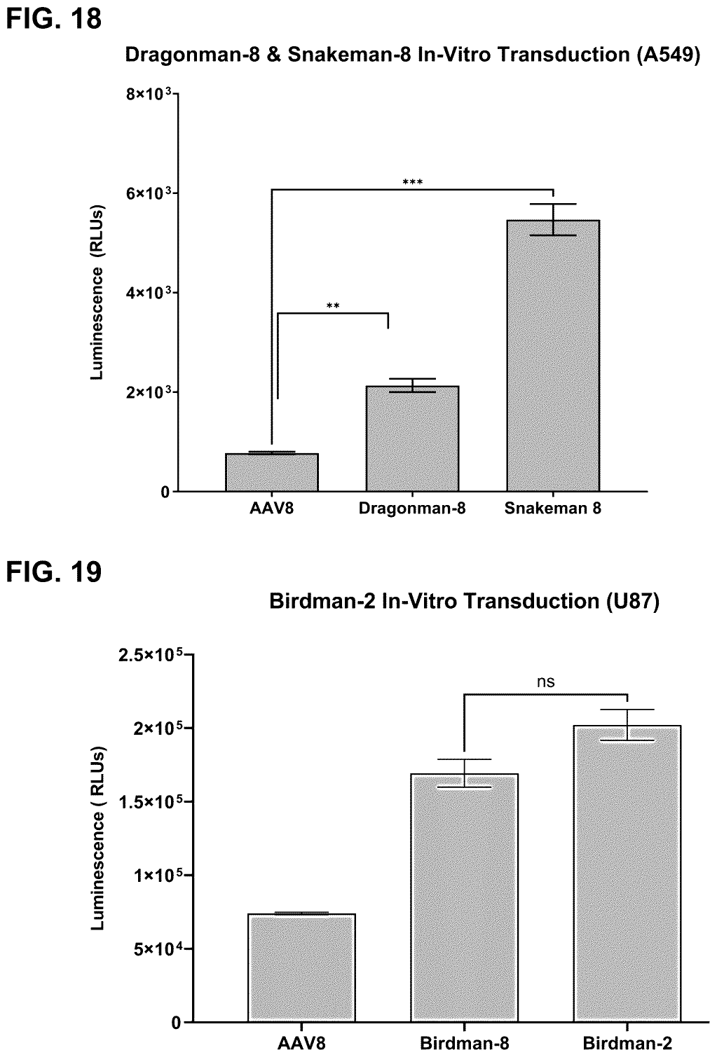

U87 cells were seeded in 96-well plates at a density of 5e4 cells/mL and grown in a 37° C. CO₂ incubator overnight. Four wells were used to establish a cell count. AAV vector packaging CBA-Fluc—was diluted in PBS (0.001% pluronic F68) to the appropriate concentration for an MOI of 5e4 vg/cell. AAV vector was then added to each well and allowed to incubate at 37° C. for 48 hours. Media was aspirated from each well and cells were then treated with 20 μL 1× passive lysis buffer (Promega) for 30 minutes. Luciferase activity was measured for a duration of 1 second with a Victor X3 plate reader (PerkinElmer) immediately following the addition of 25 μL of luciferin (Promega) to 25 μL of cell lysate. The luciferase transduction assay with Birdman-2 reveals similar transduction profiles for Birdman-8 and Birdman-2 (FIG. 19). Birdman-2 transduced U87 cells 1.19-fold greater than Birdman-8. However, this difference was not statistically significant (p=0.147).

### Example 9

### Generation of Minimal Hybrids

Finally, what minimal swaps from Birdman-8 to DA-1 were sufficient to rescue transduction in human cells was examined. To this end, three new constructs were made: DA1-VP1u-AAV8, DA1-PLA2-AAV8, and DA1-Nterm-AAV8.

To generate DA1-VP1u-AAV8, the VP1u region of DA-1 VP1 (aa 1-146) was swapped for AAV8's VP1u region (aa 1-137) (FIG. 15). To generate DA1-PLA2-AAV8, the PLA2 domain of DA-1 VP1 (aa 54-112) was swapped for AAV8's PLA2 domain (aa 45-103) (FIG. 15).

To generate DA1-Nterm-AAV8, the N-terminus of DA-1 VP1 (aa 1-51) was swapped for AAV8's N-terminus (aa 1-41) (FIG. 15). DA1-VP1u-AAV8 (SEQ ID NO:05), DA1-PLA2-AAV8 (SEQ ID NO:06), and DA1-Nterm-AAV8 (SEQ ID NO:07) AAV vectors packaging CBA-Fluc were then produced using HEK293 E1A cells and a triple transfection method. Then, U87 cells were seeded in 96-well plates at a density of 5e4 cells/mL and grown in a 37° C. CO₂ incubator overnight. Four wells were used to establish a cell count. AAV vector packaging CBA-Fluc—was diluted in PBS (0.001% pluronic F68) to the appropriate concentration for an MOI of 5e4 vg/cell. AAV vector was then added to each well and allowed to incubate at 37° C. for 48 hours. Media was aspirated from each well and cells were then treated with 20 μL 1× passive lysis buffer (Promega) for 30 minutes. Next, 20 μL firefly luciferin was added to each well. Luminescence was then measured for a duration of 1 second by the VICTOR X Plate Reader (PerkinElmer).

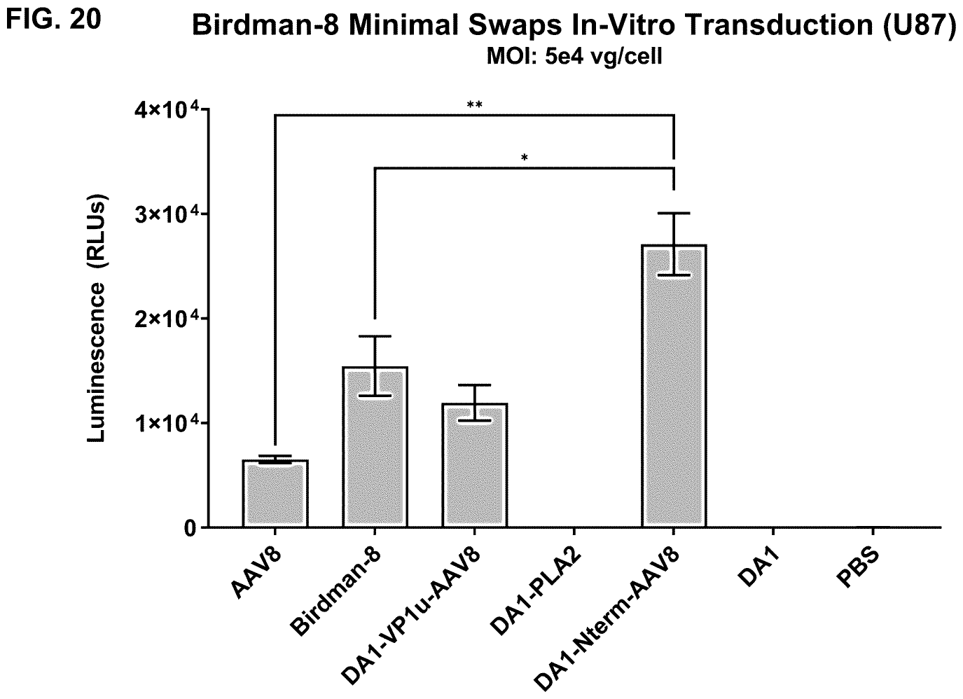

As FIG. 20 shows, DA1-PLA2-AAV8 did not transduce above background levels while Birdman-8, DA1-VP1u-AAV8, and DA1-Nterm-AAV8 all did so. DA1-VP1u-AAV8 transduced 1.83-fold greater than AAV8 (p=0.03) and at levels similar to Birdman-8 (p=0.33). DA1-Nterm-AAV8, however, transduced 4.16-fold greater than AAV8 (p=0.002) and 1.75-fold greater than Birdman-8 (p=0.02). This indicated that swapping the first 41 amino acids of AAV8 onto DA-1 was sufficient to rescue transduction in human cells and that this minimal N-terminus swap outperformed the larger VP1u and VP1/2u swaps.

### Example 10

### Generation of Portfolio Comprising Mammalian-Sauropsidan AAV Hybrids

Overall, the data disclosed herein indicated that sauropsidan AAVs (avian AAVs, reptile AAVs, etc.) were antigenically distinct from mammalian AAVs and that human transduction could be rescued in these AAVs via N-terminus swaps from a range of mammalian AAVs. This engineering approach to generating immune evasive elite AAV capsids can be applied using a broad range of dependoparvoviruses.

To this end, the VP1 sequences from 437 unique dependoparvoviruses were accessed from GenBank. The sequences were then divided into two groups according to whether their hosts belong to the mammalia or sauropsida clades. Sequences were then aligned using MUSCLE. The N-termini of mammalian dependoparvoviruses were then extracted, using an ubiquitously conserved arginine (aa 43 in AAV8) as the delimiter. Likewise, the N-termini of sauropsidan dependoparvoviruses were removed using the same arginine as a delimiter. After filtering for unique sequences, all possible combinations of sauropsidan VP1s and mammalian N-termini were generated, yielding 7923 unique novel sequences. To illustrate this process, seven representative VP1 sequences (SEQ ID NOs: 35, 41, 49, 57, 65, 73, and 81) and seven representative N-termini sequences (SEQ ID NOs: 89, 90, 91, 92, 93, 94, and 95) were selected.

| Seq. # | Representative VP1 Sequences |
|---|---|
| 35 | AAV DA-1 [YP_077183.1] |
| 41 | Snake AAV [YP_068094.1] |
| 49 | Bearded dragon parvovirus [YP_009154713.1] |
| 57 | MHH-05-2015 [YP_009552824.1] |
| 65 | Duck AAV [UAJ21443.1] |
| 73 | Goose Parvovirus Yan-1 [ALH24919.1] |
| 81 | Goose Parvovirus DY [ABP82770.1] |

| Seq. # | Representative N-Termini Sequences |
|---|---|
| 89 | AAV2_[YP_680426.1] |
| 90 | AAV4 ATCC VR-646 [NP_044927.1] |
| 91 | AAV5 [YP_068409.1] |
| 92 | AAV6 [AAB95450.1] |
| 93 | AAV8 [YP_077180.1] |
| 94 | AAV9 [UMO75524.1] |
| 95 | California sea lion AAV1 1187 [YP_009507367.1] |

The seven representative VP1 sequences and seven representative N-termini sequences were combined to yield dozens of different sequences.

| Seq. # | Combination of VP1s + N-Termini |
|---|---|
| 36 | AAV DA-1 [YP_077183.1] + AAV4 ATCC VR-646 |
| | [NP_044927.1] |
| 37 | AAV DA-1 [YP_077183.1] + AAV5 [YP_068409.1] |
| 38 | AAV DA-1 [YP_077183.1] + AAV6 [AAB95450.1] |
| 39 | AAV DA-1 [YP_077183.1] + AAV9 [UMO75524.1] |
| 40 | AAV DA-1 [YP_077183.1] + California sea lion |
| | AAV1 1187 [YP_009507367.1] |
| 42 | Snake AAV [YP_068094.1] + AAV2 [YP_680426.1]: |
| 43 | Snake AAV [YP_068094.1] + AAV4 ATCC VR-646 |
| | [NP_044927.1]: |
| 44 | Snake AAV [YP_068094.1] + AAV5 [YP_068409.1]: |
| 45 | Snake AAV [YP_068094.1] + AAV6 [AAB95450.1]: |
| 46 | Snake AAV [YP_068094.1] + AAV8 [YP_077180.1] |
| 47 | Snake AAV [YP_068094.1] + Cali. sea lion AAV9 |
| | [UMO75524.1] |
| 48 | Snake AAV [YP_068094.1] + Cali. sea lion |
| | AAV1 1187 [YP_009507367.1] |
| 50 | Bearded Dragon Parvovirus 2014 [YP_009154713.1] + |
| | AAV2 [YP_680426.1] |
| 51 | Bearded Dragon Parvovirus 2014 [YP_009154713.1] + |
| | AAV4 ATCC VR-646 [NP_044927.1] |
| 52 | Bearded Dragon Parvovirus 2014 [YP_009154713.1] + |
| | AAV5 [YP_068409.1] |
| 53 | Bearded Dragon Parvovirus 2014 [YP_009154713.1] + |
| | AAV6 [AAB95450.1] |
| 54 | Bearded Dragon Parvovirus 2014 [YP_009154713.1] + |
| | AAV8 [YP_077180.1] |
| 55 | Bearded Dragon Parvovirus 2014 [YP_009154713.1] + |
| | AAV9 [UMO75524.1] |
| 56 | Bearded Dragon Parvovirus 2014 [YP_009154713.1] + |
| | Cali. sea lion AAV1 1187 [YP_009507367.1] |
| 58 | AAV MHH-05-2015 [YP_009552824.1] + AAV2 |
| | [YP_680426.1] |
| 59 | AAV MHH-05-2015 [YP_009552824.1] + AAV4 |
| | ATCC VR-646 [NP_044927.1] |
| 60 | AAV MHH-05-2015 [YP_009552824.1] + AAV5 |
| | [YP_068409.1] |
| 61 | AAV MHH-05-2015 [YP_009552824.1] + AAV |
| | [AAB95450.1] |
| 62 | AAV MHH-05-2015 [YP_009552824.1] + AAV8 |
| | [YP_077180.1] |
| 63 | AAV MHH-05-2015 [YP_009552824.1] + AAV9 |
| | [UMO75524.1] |
| 64 | AAV MHH-05-2015 [YP_009552824.1] + |
| | Cali. sea lion AAV1 1187 [YP_009507367.1] |
| 66 | Duck AAV G003-20-Australia-2020 [UAJ21443.1] + |
| | AAV2 [YP_680426.1] |
| 67 | Duck AAV G003-20-Australia-2020 [UAJ21443.1] + |
| | AAV4 ATCC VR-646 [NP_044927.1] |
| 68 | Duck AAV G003-20-Australia-2020 [UAJ21443.1] + |
| | AAV5 [YP_068409.1] |
| 69 | Duck AAV G003-20-Australia-2020 [UAJ21443.1] + |
| | AAV6 [AAB95450.1] |
| 70 | Duck AAV G003-20-Australia-2020 [UAJ21443.1] + |
| | AAV8 [YP_077180.1] |
| 71 | Duck AAV G003-20-Australia-2020 [UAJ21443.1] + |
| | AAV9 [UMO75524.1] |
| 72 | Duck AAV G003-20-Australia-2020 [UAJ21443.1] + |
| | Cali. sea lion AAV1 1187 [YP_009507367.1] |
| 74 | Goose Parvovirus Yan-1 [ALH24919.1] + AAV2 |
| | [YP_680426.1] |
| 75 | Goose Parvovirus Yan-1 [ALH24919.1] + AAV4 |
| | ATCC VR-646 [NP_044927.1] |
| 76 | Goose Parvovirus Yan-1 [ALH24919.1] + AAV |
| | [YP_068409.1] |
| 77 | Goose Parvovirus Yan-1 [ALH24919.1] + AAV6 |
| | [AAB95450.1] |
| 78 | Goose Parvovirus Yan-1 [ALH24919.1] + AAV8 |
| | [YP_077180.1] |
| 79 | Goose Parvovirus Yan-1 [ALH24919.1] + AAV9 |
| | [UMO75524.1] |
| 80 | Goose Parvovirus Yan-1 [ALH24919.1] + |
| | Cali. sea lion AAV1 1187 [YP_009507367.1] |
| 82 | Goose Parvovirus DY [ABP82770.1] + AAV2 |
| | [YP_680426.1] |
| 83 | Goose Parvovirus DY [ABP82770.1] + AAV4 |
| | ATCC VR-646 [NP_044927.1] |
| 84 | Goose Parvovirus DY [ABP82770.1] + AAV5 |
| | [YP_068409.1] |
| 85 | Goose Parvovirus DY [ABP82770.1] + AAV6 |
| | [AAB95450.1] |
| 86 | Goose Parvovirus DY [ABP82770.1] + AAV8 |
| | [YP_077180.1] |
| 87 | Goose Parvovirus DY [ABP82770.1] + AAV9 |
| | [UM075524.1] |
| 88 | Goose Parvovirus DY [ABP82770.1] + |
| | Cali. sea lion AAV1 1187 [YP_009507367.1] |

### SUMMARY OF EXAMPLES

The compositions disclosed herein, including Birdman-8, displayed robust immune evasion. At high concentrations of anti-AAV8 serum, Birdman-8 transduction remained virtually unchanged, while that of AAV8 was nearly fully abolished in vitro. Upon “redosing” in mice previously immunized by treating with AAV9, Birdman-8 transduced multiple systemic tissues at levels significantly exceeding that of redosed AAV9 vectors.

The Examples and data disclosed herein also demonstrated that this approach could be broadly applied to re-engineer other AAV capsids of non-mammalian or non-primate origin, providing a roadmap to expand the portfolio of AAV candidates available for clinical use, with a particular focus on secondary dosing of human gene therapies.

1. An adeno-associated virus (AAV) chimeric capsid protein, comprising:
a non-mammalian VP1 or a fragment thereof; and
an N-terminus of a mammalian VP1 or a fragment thereof,
wherein the N-terminus of the mammalian VP1 replaces the N-terminus of the non-mammalian VP1.

2. The AAV chimeric capsid protein of claim 1, comprising the sequence set forth in SEQ ID NO:07.

3.-6. (canceled)

7. The AAV chimeric capsid protein of claim 1, comprising a sequence having about 90% identity to the sequence set forth in any one of SEQ ID NO: 36-SEQ ID NO:40, SEQ ID NO:42-SEQ ID NO:48, SEQ ID NO:50-SEQ ID NO: 56, SEQ ID NO:58-SEQ ID NO:64, SEQ ID NO:66-SEQ ID NO:72, SEQ ID NO: 74-SEQ ID NO:80, or SEQ ID NO:82-SEQ ID NO:88.

8. The AAV chimeric capsid protein of claim 1,
wherein the non-mammalian VP1 or a fragment thereof comprises a VP1 sequence set forth in Table 1, Table 3, Table 4, or Table 5, and
wherein an N-terminus of a mammalian VP1 or a fragment thereof comprises the N-terminus set forth in AAB95450.1, AAB95452.1, ABI16639.1, AKU89595.1, AKU89600.1, ATV81503.1, NP_044927.1, QDH44082.1, QDH44228.1, QDH44235.1, QDH44284.1, QDH44305.1, QDH44326.1, QDX47270.1, UMO75524.1, YP_009507367.1, YP_068409.1, YP_077180.1, or YP_680426.1.

9.-10. (canceled)

11. An AAV chimeric capsid protein, comprising:
(i) a mammalian VP1/VP2 (partial), and
(ii) a non-mammalian VP2 (partial)/VP3.

12.-14. (canceled)

15. The AAV chimeric capsid protein of claim 11, wherein the mammalian VP1/VP2 (partial) comprises a sequence having about 90% identity to the sequence set forth in any one of SEQ ID NO:29-SEQ ID NO:34.

16.-19. (canceled)

20. The AAV chimeric capsid protein of claim 11, wherein the non-mammalian VP2 (partial)/VP3 comprises a sequence having about 90% identity to the sequence set forth in any one of SEQ ID NO:18-SEQ ID NO:28.

21.-24. (canceled)

25. An AAV capsid comprising the AAV chimeric capsid protein of claim 1.

26. An AAV vector comprising the AAV capsid of claim 25, wherein the AAV vector evades neutralizing antibodies better than an AAV vector not having one or more copies of the AAV chimeric capsid protein.

27. (canceled)

28. The AAV vector of claim 26, further comprising a nucleic acid sequence encoding at least one transgene of interest,
wherein the transgene of interest encodes a missing, deficient, and/or mutant protein or enzyme; and
wherein the AAV vector demonstrates a higher gene transfer efficiency than an AAV vector not having one or more copies of the AAV chimeric capsid protein.

29. (canceled)

30. A method of treating a genetic disease or disorder, the method comprising:
administering to a subject in need thereof the AAV vector of claim 28
wherein, following the administering of the vector, (i) the subject does not develop an antibody response to the vector, and/or (ii) one or more aspects of cellular homeostasis and/or cellular functionality in the subject is improved and/or restored.

31.-33. (canceled)

34. The AAV chimeric capsid protein of claim 11, comprising the sequence set forth in SEQ ID NO:01.

35. The AAV chimeric capsid protein of claim 11, comprising the sequence set forth in SEQ ID NO:02.

36. The AAV chimeric capsid protein of claim 11, comprising the sequence set forth in SEQ ID NO:03.

37. The AAV chimeric capsid protein of claim 11, comprising the sequence set forth in SEQ ID NO:04.

38. The AAV chimeric capsid protein of claim 11, comprising the sequence set forth in SEQ ID NO:05.

39. An AAV capsid comprising the AAV chimeric capsid protein of claim 11.

40. An AAV vector comprising the AAV capsid of claim 39, wherein the AAV vector evades neutralizing antibodies better than an AAV vector not having one or more copies of the AAV chimeric capsid protein.

41. The AAV vector of claim 40, further comprising a nucleic acid sequence encoding at least one transgene of interest,
wherein the transgene of interest encodes a missing, deficient, and/or mutant protein or enzyme; and
wherein the AAV vector demonstrates a higher gene transfer efficiency than an AAV vector not having one or more copies of the AAV chimeric capsid protein.

42. A method of treating a genetic disease or disorder, the method comprising:
administering to a subject in need thereof the AAV vector of claim 41,
wherein, following the administering of the vector, (i) the subject does not develop an antibody response to the vector, and/or (ii) one or more aspects of cellular homeostasis and/or cellular functionality in the subject is improved and/or restored.


SEQUENCE LISTING (97 sequences)

SEQ ID NO: 1 - protein, 736 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 2 - protein, 737 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 3 - protein, 737 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 4 - protein, 735 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 5 - protein, 729 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 6 - protein, 738 aa, Artificial Sequence
  Synthetic
MSLISDAIPDWLERLVKKGVNAAADFYHLESGPPHPKANQQTQESPEKDDSRGLVLPGYKYLGPFNGLDKGEPVNAADAAALEHDKAYDQQLQAGDNPYLRYNHADAEFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 7 - protein, 729 aa, Artificial Sequence
  Synthetic
MAADGYLPDWLEDNLSEGIREWWALKPGAPKPKANQQKQDDGRGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 8 - protein, 736 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGEDPGEGTSSNAGSSAPSGVGSSIMAEGGGGPVGDAGQGADGVGNSSGNWHCDSQWLENGVVTRTTRTWVLPSYNNHLYKRIQGASGGDNNSKYFGYSTPWGYFDYNRFHCHFSPRDWQRLINNNWGIRPKAMRFRLFNIQVKEVTVQDSNTTIANNLTSTVQVFADKDYQLPYVLGSATEGTLPPFPADIYTIPQYGYCTINYNNEAVDRSAFYCLDYFPSDMLRTGNNFEFTYTFEDVPFHSMFAHNQTLDRLMNPLVDQYLWAFSSVSQVGSSGRQITYSRATKDNMAAQYRNWLPGPFFRDQQIFTGASNITQNNVFNVWEKGKQWELDNRTNLMQPGPAAATTFTGEPDRQAMQNTLAFSTTVYDQTTNTTNRDQLLITNEDEIRPTNTVGIDTWGAVPTNNQSNVTPGTRTGVNNQGALPGMVWQNRDIYLQGPIWAKIPDTDNHFHPSPLIGGFGCKHPPPQIFIKNTPVPANPAETFQTSKVVSFINQYSTGQCTVEIFWELKKETSKRWNPEIQFTSNFGNASDIQFSVSDTGSYSEPRPIGTRYLTKPL

SEQ ID NO: 9 - protein, 736 aa, Artificial Sequence; identical to SEQ ID NO: 8
  Synthetic

SEQ ID NO: 10 - protein, 736 aa, Artificial Sequence; identical to SEQ ID NO: 8
  Synthetic

SEQ ID NO: 11 - protein, 661 aa, Artificial Sequence
  Synthetic
DKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGEDPGEGTSSNAGSSAPSSVGSSVMAEGGGGPVGDAGQGADGVGNSSGNWHCDSQWLDNGVVTRTTRTWVLPSYNNHLYKRIQGPGGTDNNNKYFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQPSSGQRRLNYSRGTKNNMAVQYKNWLPGPFFRDQQIYTGASNITQNNVFNVWDKGKQWVIDNRTNLMQPGPAAATTFSGEPDRQAMQNTLAFSRTVYDQTTSTADRNQLLITNEDEIRPTNSVGIDVWGVVPNNQQSNVSPGNRTGVNNQGALPGMVWQNRDIYLQGPIWAKVPDTDNHFHPSPLIGGFGCKHPPPQIFIKNTPVPANPPETFQTAKVASFINQYSTGQCTVEIFWELKKETSKRWNPEIQFTSNFGNAADIQFSVSDTGLYSEPRPIGTRYLTKPL

SEQ ID NO: 12 - protein, 735 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKYFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSMLRTGNNFEFTYTFEDVPFHSMFAHNQTLDRLMNPLVDQYLWAFSSATQAGSSGRKLNYSRGTKTNMAVQYKNWLPGPFVRDQQIFTGASNINQNNVFNAWDKSKQWVIDNRVNMMQPGPAAATSFSGEPDRQAMQNTLAFSRTVYDQTSSTADRNQLLITNEDEIRPTNPVGIDTWGVVPNNNQSNVSAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 13 - protein, 731 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTSGGEASAAATEGSEPVAAPNMAEGGSGAMGDSAGGADGVGNASGNWHCDSQWLGDTVITKTTRTWVLPSYNNHMYQAITSGTNPDSNTQYAGYSTPWGYFDFNRFHCHFSPRDWQRLINNHWGIRPKALKFKIFNVQVKEVTTQDQTKTIANNLTSTIQIFTDNEHQLPYVLGSATEGTMPPFPSDVYALPQYGYCTMHTNQSGARFNDRSAFYCLEYFPSQMLRTGNNFEFSFEFEEVPFHSMFAHSQDLDRLMNPLLDQYLWNFSEVNGGRNAQFKKAVKGAFGAMGRNWLPGPKLLDQRVRAYSGGTDNYANWSIWSKGNKVFLKDREYLLQPGPVATTHTEDQASSVPAQNIIGIAKDPYRSGSTLAGISDIMVTDEQEIAPTNGVGWRPYGLTVTNEQNTTTAPTNAELEVLGALPGMVWQNRDIYLQGPIWAKIPKTDGKPHPSPNLGGFGLHNPPPQVFIKNTPVPADPPLEYVNQKWNSYITQYSTGQCTVEMVWELRKENSKRWNPEIQFTSNFGNRTSTMFAPNETGGYVEDRLIGTRYLTQNL

SEQ ID NO: 14 - protein, 739 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGGAEDPGEGTSSNAGAAAPASSVGSSIMAEGGGGPVGDAGQGADGVGNSSGNWHCDSQWLENGVVTRTTRTWVLPSYNNHLYKRIQGPSGGDNNNKFFGFSTPWGYFDYNRFHCHFSPRDWQRLINNNWGIRPKAMRFRLFNIQVKEVTVQDFNTTIGNNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQAGSSGRALHYSRATKTNMAAQYRNWLPGPFFRDQQIFTGASNITKNNVFSVWEKGKQWELDNRTNLMQPGPAAATTFSGEPDRQAMQNTLAFSRTVYDQTTATTDRNQILITNEDEIRPTNSVGIDAWGAVPTNNQSIVTPGTRAAVNNQGALPGMVWQNRDIYPTGTHLAKIPDTDNHFHPSPLIGRFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 15 - protein, 739 aa, Artificial Sequence; identical to SEQ ID NO: 14
  Synthetic

SEQ ID NO: 16 - protein, 733 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 17 - protein, 734 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGAGDGEPSTSTGGATPSGVGSGTMSAGGGAPMGDDHQGADGVGNASGDWHCDSQWMGDHVVTRTTRTWVLPSYNNHLYKQIASTDSPVNPDNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGQPRFQKCRAPSYADQYHNWMPGPHYRIQGWNTTNTSGNQQTGSNLWDSKSKINLGEGVTAALPGPNGMCNYSQDLGRIAWDNTMVFEKTVTETGNSDFRAPENLLTTREDETAATNPLAFKESGKVANNDQTNTVAPAVQNVNYHGLYPGSVWMDRDVYLQGPIWAKIPDVEGNIHPSPHMGGFGLKHPPPMIMIKNTPVPGNPATTFNPLKINSFITQYSTGQITVEMTWELKKESSKRWNPEIQFTNNYTSASGVDFTVGTDGSYSNSRPIGTRWLTKPL

SEQ ID NO: 18 - protein, 559 aa, Artificial Sequence
  Synthetic
GEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 19 - protein, 560 aa, Artificial Sequence
  Synthetic
GSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 20 - protein, 560 aa, Artificial Sequence
  Synthetic
GEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 21 - protein, 559 aa, Artificial Sequence
  Synthetic
GEDPGEGTSSNAGSSAPSGVGSSIMAEGGGGPVGDAGQGADGVGNSSGNWHCDSQWLENGVVTRTTRTWVLPSYNNHLYKRIQGASGGDNNSKYFGYSTPWGYFDYNRFHCHFSPRDWQRLINNNWGIRPKAMRFRLFNIQVKEVTVQDSNTTIANNLTSTVQVFADKDYQLPYVLGSATEGTLPPFPADIYTIPQYGYCTINYNNEAVDRSAFYCLDYFPSDMLRTGNNFEFTYTFEDVPFHSMFAHNQTLDRLMNPLVDQYLWAFSSVSQVGSSGRQITYSRATKDNMAAQYRNWLPGPFFRDQQIFTGASNITQNNVFNVWEKGKQWELDNRTNLMQPGPAAATTFTGEPDRQAMQNTLAFSTTVYDQTTNTTNRDQLLITNEDEIRPTNTVGIDTWGAVPTNNQSNVTPGTRTGVNNQGALPGMVWQNRDIYLQGPIWAKIPDTDNHFHPSPLIGGFGCKHPPPQIFIKNTPVPANPAETFQTSKVVSFINQYSTGQCTVEIFWELKKETSKRWNPEIQFTSNFGNASDIQFSVSDTGSYSEPRPIGTRYLTKPL

SEQ ID NO: 22 - protein, 559 aa, Artificial Sequence
  Synthetic
GEDPGEGTSSNAGSSAPSSVGSSVMAEGGGGPVGDAGQGADGVGNSSGNWHCDSQWLDNGVVTRTTRTWVLPSYNNHLYKRIQGPGGTDNNNKYFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQPSSGQRRLNYSRGTKNNMAVQYKNWLPGPFFRDQQIYTGASNITQNNVFNVWDKGKQWVIDNRTNLMQPGPAAATTFSGEPDRQAMQNTLAFSRTVYDQTTSTADRNQLLITNEDEIRPTNSVGIDVWGVVPNNQQSNVSPGNRTGVNNQGALPGMVWQNRDIYLQGPIWAKVPDTDNHFHPSPLIGGFGCKHPPPQIFIKNTPVPANPPETFQTAKVASFINQYSTGQCTVEIFWELKKETSKRWNPEIQFTSNFGNAADIQFSVSDTGLYSEPRPIGTRYLTKPL

SEQ ID NO: 23 - protein, 558 aa, Artificial Sequence
  Synthetic
GEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKYFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSMLRTGNNFEFTYTFEDVPFHSMFAHNQTLDRLMNPLVDQYLWAFSSATQAGSSGRKLNYSRGTKTNMAVQYKNWLPGPFVRDQQIFTGASNINQNNVFNAWDKSKQWVIDNRVNMMQPGPAAATSFSGEPDRQAMQNTLAFSRTVYDQTSSTADRNQLLITNEDEIRPTNPVGIDTWGVVPNNNQSNVSAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 24 - protein, 561 aa, Artificial Sequence
  Synthetic
GGAENGNATGGGAGAEGSPVSGAASIMAGGAGAPMADNNQGADGVGESAGNWHCDSKWMGDHVLTTTTRTWVLPPYNNYLYRAITSTGTDRNQAYAGFSTPWGFFDFNRFHCHFSPRDWQRLVNNHVGFRPRGMRVKIFNIQVKEVTMQDNQKTIANNLTSTVQIFADSEYLLPYVNDCASESALPPFPADIYQVPQYGYLTYHWQNANNNIEYTDRSAFYCLEYFPSSMLRTGNTFDFTFEFEDVPFHSSYAHSQFLDRIHNPLIDQYLWGIYEVSNKTAKWKKIDGSNCANRYRNWCKGPYVRDQRIRVYPGGAANYTTFSAWNAGAKAHIQDREYLFRPGISGPGTDLTNTSHVACNNNLTFAKKPQNTPANPTIDDLMITDESETLPTNPVVTKAWGAAANNAQDDGHAPAREEINDIGVLPGMCWQNRDCYLQGPIWAKIPDTENHFNPAPHMGGFGLKKPPPQIFIKPTPVPADPPATYSNAKWNSYINQYATGQITVEIVWEVQKESSKRWNPEIQFTSNFGNAFKESLNFAPNKDGDYNEPRLIGTRWLSKHL

SEQ ID NO: 25 - protein, 554 aa, Artificial Sequence
  Synthetic
SGGEASAAATEGSEPVAAPNMAEGGSGAMGDSAGGADGVGNASGNWHCDSQWLGDTVITKTTRTWVLPSYNNHMYQAITSGTNPDSNTQYAGYSTPWGYFDFNRFHCHFSPRDWQRLINNHWGIRPKALKFKIFNVQVKEVTTQDQTKTIANNLTSTIQIFTDNEHQLPYVLGSATEGTMPPFPSDVYALPQYGYCTMHTNQSGARFNDRSAFYCLEYFPSQMLRTGNNFEFSFEFEEVPFHSMFAHSQDLDRLMNPLLDQYLWNFSEVNGGRNAQFKKAVKGAFGAMGRNWLPGPKLLDQRVRAYSGGTDNYANWSIWSKGNKVFLKDREYLLQPGPVATTHTEDQASSVPAQNIIGIAKDPYRSGSTLAGISDIMVTDEQEIAPTNGVGWRPYGLTVTNEQNTTTAPTNAELEVLGALPGMVWQNRDIYLQGPIWAKIPKTDGKPHPSPNLGGFGLHNPPPQVFIKNTPVPADPPLEYVNQKWNSYITQYSTGQCTVEMVWELRKENSKRWNPEIQFTSNFGNRTSTMFAPNETGGYVEDRLIGTRYLTQNL

SEQ ID NO: 26 - protein, 562 aa, Artificial Sequence
  Synthetic
GGAEDPGEGTSSNAGAAAPASSVGSSIMAEGGGGPVGDAGQGADGVGNSSGNWHCDSQWLENGVVTRTTRTWVLPSYNNHLYKRIQGPSGGDNNNKFFGFSTPWGYFDYNRFHCHFSPRDWQRLINNNWGIRPKAMRFRLFNIQVKEVTVQDFNTTIGNNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQAGSSGRALHYSRATKTNMAAQYRNWLPGPFFRDQQIFTGASNITKNNVFSVWEKGKQWELDNRTNLMQPGPAAATTFSGEPDRQAMQNTLAFSRTVYDQTTATTDRNQILITNEDEIRPTNSVGIDAWGAVPTNNQSIVTPGTRAAVNNQGALPGMVWQNRDIYPTGTHLAKIPDTDNHFHPSPLIGRFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 27 - protein, 556 aa, Artificial Sequence
  Synthetic
GAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 28 - protein, 557 aa, Artificial Sequence
  Synthetic
GAGDGEPSTSTGGATPSGVGSGTMSAGGGAPMGDDHQGADGVGNASGDWHCDSQWMGDHVVTRTTRTWVLPSYNNHLYKQIASTDSPVNPDNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGQPRFQKCRAPSYADQYHNWMPGPHYRIQGWNTTNTSGNQQTGSNLWDSKSKINLGEGVTAALPGPNGMCNYSQDLGRIAWDNTMVFEKTVTETGNSDFRAPENLLTTREDETAATNPLAFKESGKVANNDQTNTVAPAVQNVNYHGLYPGSVWMDRDVYLQGPIWAKIPDVEGNIHPSPHMGGFGLKHPPPMIMIKNTPVPGNPATTFNPLKINSFITQYSTGQITVEMTWELKKESSKRWNPEIQFTNNYTSASGVDFTVGTDGSYSNSRPIGTRWLTKPL

SEQ ID NO: 29 - protein, 176 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQT

SEQ ID NO: 30 - protein, 176 aa, Artificial Sequence
  Synthetic
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQT

SEQ ID NO: 31 - protein, 177 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQT

SEQ ID NO: 32 - protein, 176 aa, Artificial Sequence
  Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQT

SEQ ID NO: 33 - protein, 177 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQT

SEQ ID NO: 34 - protein, 177 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQT

SEQ ID NO: 35 - protein, 738 aa, avian adenovirus
MSLISDAIPDWLERLVKKGVNAAADFYHLESGPPHPKANQQTQESPEKDDSRGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 36 - protein, 728 aa, Artificial Sequence
  Synthetic
MTDGYLPDWLEDNLSEGVREWWALQPGAPKPKANQQHQDNARGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 37 - protein, 728 aa, Artificial Sequence
  Synthetic
MSFVDHPPDWLEEVGEGLREFLGLEAGPPKPKPNQQHQDQARGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 38 - protein, 729 aa, Artificial Sequence
  Synthetic
MAADGYLPDWLEDNLSEGIREWWDLKPGAPKPKANQQKQDDGRGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 39 - protein, 729 aa, Artificial Sequence
  Synthetic
MAADGYLPDWLEDNLSEGIREWWALKPGAPQPKANQQHQDNARGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 40 - protein, 732 aa, Artificial Sequence
  Synthetic
MSSLFKEYLQSTGLVGIQSGAPKPKAGQQKQDTGSFEWKKKEDTARGLVFPGYKYLGPFNGLDKGKPVNEADAAALEHDKAYDLELKDGHNPYFEYNEADRRFQERLKDDTSFGGNLGKAIFQAKKRVLEPFGLIEQPDNTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTKPL

SEQ ID NO: 41 - protein, 726 aa, Artificial Sequence
  Synthetic
MDFLDDFFADKYKETVNELGKPVNPKPVKHISEAHSQPGSRRGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 42 - protein, 727 aa, Artificial Sequence
  Synthetic
MAADGYLPDWLEDTLSEGIRQWWKLKPGPPPPKPAERHKDDSRGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 43 - protein, 726 aa, Artificial Sequence
  Synthetic
MTDGYLPDWLEDNLSEGVREWWALQPGAPKPKANQQHQDNARGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 44 - protein, 726 aa, Artificial Sequence
  Synthetic
MSFVDHPPDWLEEVGEGLREFLGLEAGPPKPKPNQQHQDQARGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 45 - protein, 727 aa, Artificial Sequence
  Synthetic
MAADGYLPDWLEDNLSEGIREWWDLKPGAPKPKANQQKQDDGRGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 46 - protein, 727 aa, Artificial Sequence
  Synthetic
MAADGYLPDWLEDNLSEGIREWWALKPGAPKPKANQQKQDDGRGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 47 - protein, 727 aa, Artificial Sequence
  Synthetic
MAADGYLPDWLEDNLSEGIREWWALKPGAPQPKANQQHQDNARGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 48 - protein, 730 aa, Artificial Sequence
  Synthetic
MSSLFKEYLQSTGLVGIQSGAPKPKAGQQKQDTGSFEWKKKEDTARGFVVPGYRYLGPGNSLDRGKPVNKADEAAKKHDQEYDQQLKAGDNPYIKYNHADEQFQKDLQGDTSLAGNAANALFQGKKTLLAPLGLVETPVGKTSEKHKLDEYYPKAKKAKQGLQIPAPPKGGEEEATSSQSGGSPAGSDTSGTSVMATGGGGPMADDNQGAEGVGNSSGDWHCDTKWMGDHVITKSTRTWVLPTYGNHLYGPINFDGTTGSGANAAYAGYKTPWGYFDFNRFHCHFSPRDWQRLINNHTGIRPKGLKIKVFNVQVKEVTTQDSTKTIANNLTSTVQIFADENYDLPYVLGSATQGTFPPFPNDVFMLPQYAYCTLQGNSGKFVDRSAFYCLEYFPSQMLRTGNNFEFQFKFEEVPFHSGWAQSQSLDRLMNPLLDQYLIGDYGTDASGNLIYHRAGPNDLNEFYKNWAPAPYECIQNINSSDNTKNANSINGSNSTNKWGLQGRQAWDAPGFVQASTYEGAAAGQSLLNGVLTFDKSSATTSSPAATAVNRTIEDEIQGTNNFGNARNNIVAINQQTKGTNPTTGSTSQFETMPGMVWSNRDIYLQGPIWAKIPNTDGHFHPSPRMGGFGLKHPPPMILIKNTPVPADPPTTFNPMPQTSFITEYSTGQVTVEMLWEVQKESSKRWNPEVQFTSNFGTSDPAVDGIPFGINNLGTYVESRPIGTRYISKHL

SEQ ID NO: 49 - protein, 725 aa, Artificial Sequence
  Synthetic
MDFLDFLLGEKYEETAKELGKPINPPPVQQISHADSRRGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 50 - protein, 730 aa, Artificial Sequence
  Synthetic
MAADGYLPDWLEDTLSEGIRQWWKLKPGPPPPKPAERHKDDSRGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 51 - protein, 729 aa, Artificial Sequence
  Synthetic
MTDGYLPDWLEDNLSEGVREWWALQPGAPKPKANQQHQDNARGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 52 - protein, 729 aa, Artificial Sequence
  Synthetic
MSFVDHPPDWLEEVGEGLREFLGLEAGPPKPKPNQQHQDQARGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 53 - protein, 730 aa, Artificial Sequence
  Synthetic
MAADGYLPDWLEDNLSEGIREWWDLKPGAPKPKANQQKQDDGRGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 54 - protein, 730 aa, Artificial Sequence
  Synthetic
MAADGYLPDWLEDNLSEGIREWWALKPGAPKPKANQQKQDDGRGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 55 - protein, 730 aa, Artificial Sequence
  Synthetic
MAADGYLPDWLEDNLSEGIREWWALKPGAPQPKANQQHQDNARGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 56 - protein, 733 aa, Artificial Sequence
  Synthetic
MSSLFKEYLQSTGLVGIQSGAPKPKAGQQKQDTGSFEWKKKEDTARGLVVPGYKYLGPFNGLDKGEPVNAADAAALEHDKAYNELLEAGDNPYIKYNHADAVFQERLQGDTSLGGNAANAVFQFKKRLLEPFGAVEQPQPEKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGSRYLSKHL

SEQ ID NO: 57 - protein, 738 aa, Artificial Sequence
  Synthetic
MSAADSVPDWLENFVRKHIVNPVAEFAHLEAGAPQPKPNQQHQDRGGTKDDSRGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 58 - protein, 728 aa, Artificial Sequence
  Synthetic
MAADGYLPDWLEDTLSEGIRQWWKLKPGPPPPKPAERHKDDSRGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 59 - protein, 727 aa, Artificial Sequence
  Synthetic
MTDGYLPDWLEDNLSEGVREWWALQPGAPKPKANQQHQDNARGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 60 - protein, 727 aa, Artificial Sequence
  Synthetic
MSFVDHPPDWLEEVGEGLREFLGLEAGPPKPKPNQQHQDQARGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 61 - protein, 728 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 62 - protein, 728 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 63 - protein, 728 aa, Artificial Sequence
  Synthetic
MAADGYLPDWLEDNLSEGIREWWALKPGAPQPKANQQHQDNARGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 64 - protein, 731 aa, Artificial Sequence
  Synthetic
MSSLFKEYLQSTGLVGIQSGAPKPKAGQQKQDTGSFEWKKKEDTARGLVLPGYKYLGPFNGLDKGEPVNAADAAALEHDKAYDQQLQAGDNPYLKYNHADAEFQEALKDDTSFGGNLGKAVFQAKKRVLEPFGLAEDGKTAPTNERRKENIDDYYPKRKKAKAGEEKPPSTDAVEGAGDGEPSTSTGGETPSGTQSNTMSAGGGAPMGDDQQGADGVGNSSGNWHCDSTWLDNLVITKSTRTWVLPSYNNHIYKRVSNTGGDNSYFGFSTPWGYFDFNRFHCHFSPRDWQRLINNNWGIRPKALKFKLFNIQVKEVTTQDSTKTVANNLTSTIQVFADSEYQLPYVVGNASEGCLPPFPADVFVLPQYGYLTLDNNGNSVDRSAFYCLEYFPSQMLRTGNNFEFTYEFEKVPFHSMFAHNQSLSRLMNPLVDQYLYYFSNVSGPNNAAQIRYDKGRKEDIAGQCRNWLPGPFWQNQSVRLDNANNNPKWDYWANSNRVRLDGKLYSVNPGIPQATEATQNPYNQYPTQSTLVFEKKPGGNPTGVDPNNLNLTKDEEIRTTNPVAYAVSGTVTGTSAINQNNGASQTPTASDVDILGAMPGMVWQNRDIYLQGPIWAKIPSTDNHFHPCPLMGGFGFKHPPPQILIKNTPVPSDPAGFSATKFNSFITQYSTGQVTVEIMWELQKETSKRWNPEIQFTSNFNATQELQFAPNVSGDYEEPRAIGSRYLTKPL

SEQ ID NO: 65 - protein, 737 aa, Artificial Sequence
  Synthetic
MSAADSVPDWLENFVRKHIVNPVAEFAHLEAGAPKPKPHQQHQNRSDAKDDSRGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 66 - protein, 727 aa, Artificial Sequence
  Synthetic
MAADGYLPDWLEDTLSEGIRQWWKLKPGPPPPKPAERHKDDSRGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 67 - protein, 726 aa, Artificial Sequence
  Synthetic
MTDGYLPDWLEDNLSEGVREWWALQPGAPKPKANQQHQDNARGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 68 - protein, 726 aa, Artificial Sequence
  Synthetic
MSFVDHPPDWLEEVGEGLREFLGLEAGPPKPKPNQQHQDQARGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 69 - protein, 727 aa, Artificial Sequence
  Synthetic
MAADGYLPDWLEDNLSEGIREWWDLKPGAPKPKANQQKQDDGRGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 70 - protein, 727 aa, Artificial Sequence
  Synthetic
MAADGYLPDWLEDNLSEGIREWWALKPGAPKPKANQQKQDDGRGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 71 - protein, 727 aa, Artificial Sequence
  Synthetic
MAADGYLPDWLEDNLSEGIREWWALKPGAPQPKANQQHQDNARGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 72 - protein, 730 aa, Artificial Sequence
  Synthetic
MSSLFKEYLQSTGLVGIQSGAPKPKAGQQKQDTGSFEWKKKEDTARGLVLPGYKYLGPLNGLDKGEPVNAADAAALAHDKAYDEQLKAGDNPYLKYNHADAEFQEELKDDTSFGGNLGKAVFQAKKRVLEPFGLAEDGKTAPTGNRRRENIDDYYPKRKKAKAGEEKPPSTQEVEGAGDGEPSTSTGGATPSGVGSDTMSAGGGAPMGDANQGADGVGNASGDWHCDSQWVGDHVVTRTTRTWVLPSYNNHLYKQIASANSNADNAYFGYSTPWGYFDFNRFQCHFSPRDWQRLVNNNWGFRPKSLKVKIFNIQVKEVTQQDSTTTIANNLTSTVQVFADSDYQLPYVLGNATQGTLPPFPPDVFMLPQYAYATLNSGNGPATNGRSAFYCLEYFPSQMLRTGNNFEMSFEFESVPFHTGYAPNQFLNRLSNPLIDQYLYRMIGLNTGEPRFQKCNAGSYADQYHNWMPGPHYRIQGWNTTNTAGNQQSGSNLWDSKSKINLGEGVNAAIPGPNGMCNYSQDLGRIAWDNTMVFEKTVTENGNTDFRAPENLLTTREDETAATNPLAFKESGKVANNDQSNSVAPEVQNVHYHGLYPGSVWMDRDVYLQGPIWAKIPDVEGNIHPSPHMGGFGLKNPPPMIMIKNTPVPGNPATSFSPLKINSFITQYSTGQITVEMTWELKKESSKRWNPEIQFTNNYASDTGVDFTVGTDGSYSNSRPIGTRWLTKPL

SEQ ID NO: 73 - protein, 732 aa, goose parvovirus
MSTFLDSFGEWYETAAASWRNLKAGAPHPKPNQQTQSLSPAKEPERRDNNRGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 74 - protein, 724 aa, Artificial Sequence
  Synthetic
MAADGYLPDWLEDTLSEGIRQWWKLKPGPPPPKPAERHKDDSRGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 75 - protein, 723 aa, Artificial Sequence
  Synthetic
MTDGYLPDWLEDNLSEGVREWWALQPGAPKPKANQQHQDNARGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 76 - protein, 723 aa, Artificial Sequence
  Synthetic
MSFVDHPPDWLEEVGEGLREFLGLEAGPPKPKPNQQHQDQARGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 77 - protein, 724 aa, Artificial Sequence
  Synthetic
MAADGYLPDWLEDNLSEGIREWWDLKPGAPKPKANQQKQDDGRGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 78 - protein, 724 aa, Artificial Sequence
  Synthetic
MAADGYLPDWLEDNLSEGIREWWALKPGAPKPKANQQKQDDGRGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 79 - protein, 724 aa, Artificial Sequence
  Synthetic
MAADGYLPDWLEDNLSEGIREWWALKPGAPQPKANQQHQDNARGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 80 - protein, 727 aa, Artificial Sequence
  Synthetic
MSSLFKEYLQSTGLVGIQSGAPKPKAGQQKQDTGSFEWKKKEDTARGFVLPGYKYLGPGNGLDKGPPVNKADSVALEHDKAYDQQLKAGDNPYIKFNHADQDFIDSLQDDQSFGGNLGKAVFQAKKRILEPFGLVEDPVNTAPAKKNTGKLTDHYPVVKKPKLTEEVSAGGGTSAVQDGGATAEGTEPVAASEMAEGGGGALGDASGGADGVGNASGNWHCDSQWMGNTVITKTTRTWVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSSRNAQFKKAVKGAYGTMGRNWLPGPKFLDQRVRAYTGGTDNYANWNIWSNGNKVNLKDRQYLLQPGPVSATHTEAEASSIPAQNILGIAKDPYRSGSTAAGISDIMVTDEQEVAPTNGVGWKPYGRTVTNEQNTTTAPTSSDLDVLGALPGMVWQNRDIYLQGPIWAKIPKTDGKFHPSPNLGGFGLHNPPPQVFIKNTPVPADPPVEYVHQKWNSYITQYSTGQCTVEMVWELRKENSKKWNPEIQVTSNFSNRTSIMFAPNETGGYVEDRLIGTRYLTQNL

SEQ ID NO: 81 - protein, 732 aa, goose parvovirus
MSTFLDSFEEWYETAAASWRHLKAGAPKPKPNQQSQSVSTDRKPQRKDNNRGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 82 - protein, 724 aa, Artificial Sequence
  Synthetic
MAADGYLPDWLEDTLSEGIRQWWKLKPGPPPPKPAERHKDDSRGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 83 - protein, 723 aa, Artificial Sequence
  Synthetic
MTDGYLPDWLEDNLSEGVREWWALQPGAPKPKANQQHQDNARGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 84 - protein, 723 aa, Artificial Sequence
  Synthetic
MSFVDHPPDWLEEVGEGLREFLGLEAGPPKPKPNQQHQDQARGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 85 - protein, 724 aa, Artificial Sequence
  Synthetic
MAADGYLPDWLEDNLSEGIREWWDLKPGAPKPKANQQKQDDGRGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 86 - protein, 724 aa, Artificial Sequence
  Synthetic
MAADGYLPDWLEDNLSEGIREWWALKPGAPKPKANQQKQDDGRGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 87 - protein, 724 aa, Artificial Sequence
  Synthetic
MAADGYLPDWLEDNLSEGIREWWALKPGAPQPKANQQHQDNARGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 88 - protein, 727 aa, Artificial Sequence
  Synthetic
MSSLFKEYLQSTGLVGIQSGAPKPKAGQQKQDTGSFEWKKKEDTARGFVLPGYKYLGPGNGLDKGPPVNKADSVALEHDKAYDQQLKAGDNPYIKFNHADQEFIDSLQDDRSFGGNLGKAVFQAKKRILEPLGLVEEPVNTAPAKKSSGKLTDHYPIVKKPKLSEENSPSPSNSGGEASAAATEGSEPVAAPNMAEGGSGAMGDSAGGADGVGNASGNWHCDSQWLGDTVITKTTRTCVLPSYNNHIYKAITSGTSQDANVQYAGYSTPWGYFDFNRFHCHFSPRDWQRLINNHWGIRPKSLKFKIFNVQVKEVTTQDQTKTIANNLTSTIQVFTDDEHQLPYVLGSATEGTMPPFPSDVYALPQYGYCTMHTNQNGARFNDRSAFYCLEYFPSQMLRTGNNFEFTFDFEEVPFHSMFAHSQDLDRLMNPLVDQYLWNFNEVDSNRNAQFKKAVKGAYGTMGRNWLPGPKFLDQRVRAYPGGTDNYASWNIWSNGNKVNLKDRQYLLQPGPVSATHTEAEASSIPAQNILGLAKDPYRSGSTAAGISDIMVTDEQEVAPTNGVGWKPYGKTVTNEQNTTRAPTSSDLDVLGALPGMVWQNRDIYLQGPIWAKIPKTDGKFHPSPNLGGFGLHNPPPQVFIKNTPVPADPPLEYVNQKWNSYITQYSTGQCTVEMVWELRKENSKRWNPEIQFTSNFGNRTSTMFAPSETGGYVEDRLIGTRYLTQNL

SEQ ID NO: 89 - protein, 735 aa, Artificial Sequence [sequence omitted]

SEQ ID NO: 90 - protein, 734 aa, Artificial Sequence
  Synthetic
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 91 - protein, 724 aa, Artificial Sequence
  Synthetic
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 92 - protein, 736 aa, Artificial Sequence
  Synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 93 - protein, 738 aa, Artificial Sequence
  Synthetic
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 94 - protein, 736 aa, Artificial Sequence
  Synthetic
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 95 - protein, 718 aa, Artificial Sequence
  Synthetic
MSSLFKEYLQSTGLVGIQSGAPKPKAGQQKQDTGSFEWKKKEDTARGLVLPGYKYLGPFNGLERGEPVNAADAAAQRHDRQYDRILQQGGNPYLTYNHADREFQEELQSDESFGGNLGKAVFQAKKRILEPLGLVEEDPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQNQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 96 - DNA, 25 bp, Artificial Sequence
  Synthetic
aacatgctacgcagagagggagtgg

SEQ ID NO: 97 - DNA, 29 bp, Artificial Sequence
  Synthetic
catgagacaaggaacccctagtgatggag